(19) United States
(12) Patent Application Publication
(10) Pub. No.: US 2025/0089818 A1
(43) Pub. Date: Mar. 20, 2025

(54) Smart Bra with Optical Sensors for Breast Cancer Screening

(71) Applicant: Robert A. Connor, Wyoming, MN (US)

(72) Inventor: Robert A. Connor, Wyoming, MN (US)

(73) Assignee: Holovisions LLC, Ham Lake, MN (US)

(21) Appl. No.: 18/964,893

(22) Filed: Dec. 2, 2024

Related U.S. Application Data
(63) Continuation-in-part of application No. 18/666,352, filed on May 16, 2024.
(63) Continuation-in-part of application No. 18/237,231, filed on Aug. 23, 2023.
(63) Continuation-in-part of application No. 18/096,748, filed on Jan. 13, 2023, now Pat. No. 12,201,402.
Application No. 18/096,748 is a continuation-in-part of application No. 18/237,231, filed on Aug. 23, 2023.
Application No. 18/237,231 is a continuation-in-part of application No. 18/096,748, filed on Jan. 13, 2023, now Pat. No. 12,201,402.
Application No. 18/096,748 is a continuation-in-part of application No. 17/897,182, filed on Aug. 28, 2022, now Pat. No. 11,950,881.
Application No. 17/897,182 is a continuation-in-part of application No. 16/933,138, filed on Jul. 20, 2020, now abandoned.
(60) Provisional application No. 63/648,156, filed on May 15, 2024.
(60) Provisional application No. 63/648,156, filed on May 15, 2024.
(60) Provisional application No. 62/879,485, filed on Jul. 28, 2019.

(51) Int. Cl. A41C 3/00; A61B 5/00

(52) U.S. Cl. CPC A41C 3/0064; A61B 5/0091; A61B 5/6804

(57) ABSTRACT

A smart bra uses optical modules with light emitters and light receivers to screen for breast cancer. Light is transmitted through breast tissue and analyzed to detect, locate, image, and/or characterize abnormal breast tissue. Each optical module can have two light emitters which emit light at different wavelengths and a light receiver. An optical module can also include a movable light guide which reflects and scans beams of light from a light emitter through breast tissue at different angles.

### CROSS-REFERENCE TO RELATED APPLICATIONS

This application is a continuation-in-part of U.S. application Ser. No. 18/666,352 filed on 2024 May 16. This application claims the priority benefit of U.S. provisional application 63/648,156 filed on 2024 May 15. This application is a continuation-in-part of U.S. application Ser. No. 18/237,231 filed on 2023 Aug. 23. This application is a continuation-in-part of U.S. application Ser. No. 18/096,748 filed on 2023 Jan. 13. U.S. application Ser. No. 18/666,352 claimed the priority benefit of U.S. provisional application 63/648,156 filed on 2024 May 15. U.S. application Ser. No. 18/666,352 was a continuation-in-part of U.S. application Ser. No. 18/237,231 filed on 2023 Aug. 23. U.S. application Ser. No. 18/237,231 was a continuation-in-part of U.S. application Ser. No. 18/096,748 filed on 2023 Jan. 13. U.S. application Ser. No. 18/096,748 was a continuation-in-part of U.S. application Ser. No. 17/897,182 filed on 2022 Aug. 28 which issued as U.S. patent Ser. No. 11/950,881 on 2024 Apr. 9. U.S. application Ser. No. 17/897,182 was a continuation-in-part of U.S. application Ser. No. 16/933,138 filed on 2020 Jul. 20. U.S. application Ser. No. 16/933,138 claimed the priority benefit of U.S. provisional application 62/879,485 filed on 2019 Jul. 28. The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to wearable devices for medical imaging and diagnosis.

### Introduction

Early detection of breast cancer is vital. Current methods for breast cancer detection include x-ray mammography, MRI, and ultrasound. However, these methods have limitations. X-ray mammography causes exposure to ionizing radiation and is not very mobile, MRI equipment is expensive and not very mobile, and ultrasound can be labor-intensive and have low sensitivity. Optical imaging and spectroscopic analysis is another method for breast cancer detection which can address these limitations.

Optical imaging and spectroscopic analysis of breast tissue takes advantage of differences in light absorption and scattering by normal tissue vs. abnormal tissue. There are several biomarkers which are present in different concentrations in abnormal tissue. These biomarkers include deoxygenated hemoglobin, oxygenated hemoglobin, lipids, collagen, oxygen, and water. When near-infrared light is transmitted through and/or reflected from breast tissue, changes in light transmission caused by these biomarkers can be used to detect and image abnormal breast tissue.

With continuous wave (CW) optical analysis methods, light emitters direct light with constant intensity into and/or onto breast tissue. With frequency domain (FD) optical analysis methods, light emitters direct modulated light into and/or onto breast tissue. With time domain (TD) optical analysis methods, light emitters direct pulses of light into and/or onto breast tissue.

Variations on optical imaging and spectroscopic analysis methods in the prior art include: Diffuse Optical Imaging (DOI), Diffuse Optical Spectroscopic Imaging (DOSI), Diffuse Optical Spectroscopy (DOS), Diffuse Optical Tomography (DOT), Frequency-Domain Photon Migration (FDPM), Functional Near-Infrared Spectroscopy (fNIRS), Near-Infrared Spectroscopy (NIRS), Raman spectroscopy, Reflectance Diffuse Optical Tomography (RDOT), Transillumination Imaging (TI), and/or Transmittance Diffuse Optical Tomography (TDOT).

Although optical methods for detection of breast cancer are promising, there some limitations with devices and methods in the prior art. One challenge is the difficulty of using optical methods to scan greater tissue depth because light is increasingly scattered through larger spans of tissue. There remains a need for innovative optical devices and methods to detect breast cancer which solve these challenges.

### Review of the Relevant Art

In the patent literature, U.S. patent application 20050043596 (Chance, Feb. 24, 2005, “Optical Examination Device, System and Method”) discloses a brush-form optical coupler with freely extending fiber end portions, sized and positioned to make optical contact with a subject, examination, and monitoring systems utilizing one or more of such couplers. U.S. patent application 20060058683 (Chance, Mar. 16, 2006, “Optical Examination of Biological Tissue Using Non-Contact Irradiation and Detection”) and U.S. Pat. No. 7,904,139 (Chance, Mar. 8, 2011, “Optical Examination of Biological Tissue Using Non-Contact Irradiation and Detection”) disclose an optical system for examination of biological tissue which includes a light source, a light detector, optics and electronics. Sometimes inventions are the result of serendipitous insights; in this case, optical scanning of biological tissue may actually have been invented by chance.

U.S. Pat. No. 6,081,322 (Barbour, Jun. 27, 2000, “NIR Clinical Opti-Scan System”) and RE38800 (Barbour, Sep. 20, 2005, “NIR Clinical Opti-Scan System”) disclose three-dimensional optical imaging techniques for the detection and three-dimensional imaging of absorbing and/or scattering structures in complex random media, such as human body tissue, by detecting scattered light. U.S. patent application 20150182121 (Barbour, Jul. 2, 2015, “Low-Cost Screening System for Breast Cancer Detection”) discloses a portable and wearable tumor detector including a brassier and devices for optical tomography. U.S. patent application publication 20150119665 (Barbour et al., Apr. 30, 2015, “Self-Referencing Optical Measurement for Breast Cancer Detection”) and U.S. Pat. No. 9,724,489 (Barbour et al., Aug. 8, 2017, “Self-Referencing Optical Measurement for Breast Cancer Detection”) disclose obtaining optical data from a pair of breasts, employing a simultaneous bilateral referencing protocol, and employing a self-referencing data analysis method.

U.S. patent applications 20100292569 (Hielscher et al., Nov. 18, 2010, “Systems and Methods for Dynamic Imaging of Tissue Using Digital Optical Tomography”) and 20150223697 (Hielscher et al., Aug. 13, 2015, “Systems and Methods for Dynamic Imaging of Tissue Using Digital Optical Tomography”) disclose methods for imaging tissue using diffuse optical tomography including directing a amplitude modulated optical signals from optical signal sources. U.S. patent application 20140330116 (Hielscher et al., Nov. 6, 2014, “Systems and Methods for Simultaneous Multi-Directional Imaging for Capturing Tomographic Data”) discloses devices, systems, and method for tomographic imaging in which light transmitted and backscattered surface light is imaged by an optical system that minimizes reflection back to the target object. U.S. patent applications 20130289394 (Hielscher et al., Oct. 31, 2013, “Dynamic Optical Tomographic Imaging Devices Methods and Systems”), 20170027480 (Hielscher et al., Feb. 2, 2017, “Dynamic Optical Tomographic Imaging Devices Methods and Systems”), and 20190282134 (Hielscher et al., Sep. 19, 2019, “Dynamic Optical Tomographic Imaging Devices Methods and Systems”), and U.S. patent Ser. No. 10/178,967 (Hielscher et al., Jan. 15, 2019, “Dynamic Optical Tomographic Imaging Devices Methods and Systems”) disclose an optical tomographic systems for acquiring and displaying dynamic data representing changes in a target tissue sample to external provocation. U.S. patent applications 20130338496 (Hielscher et al., Dec. 19, 2013, “Medical Imaging Devices, Methods, and Systems”) and 20140088415 (Hielscher et al., Mar. 27, 2014, “Medical Imaging Devices, Methods, and Systems”) disclose devices, methods, and systems for generating optical tomographic data including volumetric and surface geometric data.

U.S. patent application publication 20140236003 (Hielscher et al., Aug. 21, 2014, “Interfacing Systems, Devices, and Methods for Optical Imaging”) discloses an imaging interface with a plurality of concentric rings for diffuse optical tomography of breast tissue. U.S. patent applications 20140243681 (Hielscher et al., Aug. 28, 2014, “Compact Optical Imaging Devices, Systems, and Methods”) and 20190239751 (Hielscher et al., Aug. 8, 2019, “Compact Optical Imaging Devices, Systems, and Methods”), and U.S. patent Ser. No. 10/111,594 (Hielscher et al., Oct. 30, 2018, “Compact Optical Imaging Devices, Systems, and Methods”) disclose a handheld optical imaging system with a plurality of detectors. U.S. patent application 20150286785 (Hielscher et al., Oct. 8, 2015, “Systems, Methods, and Devices for Image Reconstruction Using Combined PDE-Constrained and Simplified Spherical Harmonics Algorithm”) and U.S. Pat. No. 9,495,516 (Hielscher et al., Nov. 15, 2016, “Systems, Methods, and Devices for Image Reconstruction Using Combined PDE-Constrained and Simplified Spherical Harmonics Algorithm”) disclose systems, methods, and devices for image reconstruction using combined PDE-constrained and simplified spherical harmonics (SPN) algorithms. U.S. patent Ser. No. 10/376,150 (Hielscher et al., Aug. 13, 2019, “Interfacing Systems, Devices, and Methods for Optical Imaging”) discloses an imaging interface for diffuse optical tomography of breast with a plurality of concentric rings.

U.S. patent application publication 20140236021 (Islam, Aug. 21, 2014, “Near-Infrared Super-Continuum Lasers for Early Detection of Breast and Other Cancers”) and U.S. Pat. No. 9,993,159 (Islam, Jun. 12, 2018, “Near-Infrared Super-Continuum Lasers for Early Detection of Breast and Other Cancers”) disclose a system and method using near-infrared or short-wave infrared light sources for early detection and monitoring of breast cancer. U.S. patent application publication 20180289264 (Islam. Oct. 11, 2018, “High Signal-to-Noise Ratio Light Spectroscopy of Tissue”) discloses a diagnostic system which delivers an optical beam to a nonlinear element that broadens a spectrum of the first optical beam to at least 10 nanometers through a nonlinear effect in the nonlinear element. U.S. patent application 20210038083 (Islam. Feb. 11, 2021, “Multi-Wavelength Wearable Device for Non-Invasive Blood Measurements in Tissue”) discloses a system for measuring one or more physiological parameters with a wearable device that includes a light source comprising a driver and semiconductor sources that generate an output optical light.

U.S. patent application publication 20090005692 (Intes et al., Jan. 1, 2009, “Optical Imaging Method for Tissue Characterization”) and U.S. Pat. No. 8,565,862 (Intes et al., Oct. 22, 2013, “Optical Imaging Method for Tissue Characterization”) disclose a method for detecting and characterizing abnormalities within biological tissue by characterizing optical properties of the tissue. U.S. patent application publication 20180070891 (Jepsen, Mar. 15, 2018, “Imaging With Infrared Imaging Signals”) discloses using an infrared imaging signal to image tissue. U.S. patent application publication 20180335753 (Jepsen et al., Nov. 22, 2018, “Co-Located Imaging and Display Pixel”) discloses an optical transformation engine coupled between an image pixel and a display pixel. U.S. patent application publication 20190072897 (Jepsen et al., 317/2019, “Applications of Diffuse Medium Imaging”) discloses methods and an apparatus for imaging translucent materials.

U.S. Pat. No. 9,314,218 (Stearns et al., Apr. 19, 2016, “Integrated Microtomography and Optical Imaging Systems”) and U.S. Pat. No. 10,130,318 (Steams et al., Nov. 20, 2018, “Integrated Microtomography and Optical Imaging Systems”) disclose an integrated microtomography and optical imaging system with a rotating table that supports an imaging object, an optical stage, and separate optical and microtomography imaging systems. U.S. Pat. No. 9,770,220 (Stearns et al., Sep. 26, 2017, “Integrated Microtomography and Optical Imaging Systems”) discloses a rotating table that supports an imaging object, an optical stage, and separate optical and microtomography imaging systems. U.S. patent application 20170209083 (Zarandi et al., 2017, “Hand-Held Optical Scanner for Real-Time Imaging of Body Composition and Metabolism”) and U.S. patent Ser. No. 10/653,346 (Zarandi et al., May 19, 2020, “Hand-Held Optical Scanner for Real-Time Imaging of Body Composition and Metabolism”) disclose a handheld system for diffuse optical spectroscopic imaging of human tissue.

U.S. patent application 20060173352 (Lilge et al., 2006, “Optical Transillumination and Reflectance Spectroscopy to Quantify Disease Risk”) discloses a method of illuminating tissue of a mammal with light having wavelengths covering a pre-selected spectral range, detecting light transmitted through, or reflected from, the volume of selected tissue, and obtaining a spectrum of the detected light. U.S. patent application 20200116630 (Zhu, 2020, “Compact Guided Diffuse Optical Tomography System for Imaging a Lesion Region”) discloses a compact diffuse optical tomography system with laser diodes and a laser diode driver board. U.S. Pat. No. 5,876,339 (Lemire, Mar. 2, 1999, “Apparatus for Optical Breast Imaging”) discloses an optical breast imager with an adjustable volume which encloses a patient's breast.

U.S. Pat. No. 5,999,836 (Nelson et al., Dec. 7, 1999, “Enhanced High Resolution Breast Imaging Device and Method Utilizing Non-Ionizing Radiation of Narrow Spectral Bandwidth”) and U.S. Pat. No. 6,345,194 (Nelson et al., Feb. 5, 2002, “Enhanced High Resolution Breast Imaging Device and Method Utilizing Non-Ionizing Radiation of Narrow Spectral Bandwidth”) disclose breast imaging using collimated non-ionizing radiation in the near ultraviolet, visible, infrared, and microwave regions. U.S. Pat. No. 6,240,309 (Yamashita et al., May 29, 2001, “Optical Measurement Instrument for Living Body”), U.S. Pat. No. 6,640,133 (Yamashita et al., Oct. 28, 2003, “Optical Measurement Instrument for Living Body”), and U.S. Pat. No. 7,142,906 (Yamashita et al., Nov. 28, 2006, “Optical Measurement Instrument for Living Body”) disclose an optical measurement instrument which applies visible-infrared light to several positions on a patient.

U.S. patent application 20020045833 (Wake et al., Apr. 18, 2002, “Medical Optical Imaging Scanner Using Multiple Wavelength Simultaneous Data Acquisition for Breast Imaging”) discloses a scanner for a medical optical imaging device with an illumination source which directs emitted light into a breast positioned below a support surface. U.S. Pat. No. 6,571,116 (Wake et al., May 27, 2003, “Medical Optical Imaging Scanner Using Multiple Wavelength Simultaneous Data Acquisition for Breast Imaging”) and U.S. Pat. No. 6,738,658 (Wake et al., May 18, 2004, “Medical Optical Imaging Scanner Using Multiple Wavelength Simultaneous Data Acquisition for Breast Imaging”) disclose a medical optical imaging device with an illumination source that directs emitted light into a breast positioned below a support surface.

U.S. patent application publication 20040092826 (Corbeil et al., May 13, 2004, “Method and Apparatus for Optical Imaging”) and U.S. Pat. No. 7,809,422 (Corbeil et al., Oct. 5, 2010, “Method and Apparatus for Optical Imaging”) disclose a platform with a cavity into which one of the person's breasts is suspended for optical imaging. U.S. patent application publication 20070287897 (Faris, Dec. 13, 2007, “Optical Vascular Function Imaging System and Method for Detection and Diagnosis of Cancerous Tumors”) discloses an in-vivo optical imaging system and method of identifying unusual vasculature associated with tumors. U.S. Pat. No. 8,027,711 (Jones et al., Sep. 27, 2011, “Laser Imaging Apparatus with Variable Patient Positioning”) discloses a tabletop to support a patient in front-down position and an opening to permit a breast of the patient to be vertically pendant below the tabletop.

U.S. Pat. No. 8,224,426 (Lilge et al., Jul. 17, 2012, “Optical Transillumination and Reflectance Spectroscopy to Quantify Disease Risk”) discloses spectroscopic tissue volume measurements with non-ionizing radiation to detect pre-disease transformations in tissue. U.S. patent application publication 20160066811 (Mohamadi, Mar. 10, 2016, “Handheld and Portable Scanners for Millimeter Wave Mammography and Instant Mammography Imaging”) discloses an array of ultra-wide band radio frequency sensors for breast imaging. U.S. Pat. No. 9,513,276 (Tearney et al., Dec. 6, 2016, “Method and Apparatus for Optical Imaging via Spectral Encoding”) disclose a method, apparatus and arrangement for obtaining information associated with a sample such as a portion of an anatomical structure. U.S. patent application publication 20170007187 (Breneisen et al., Jan. 12, 2017, “Cancer Detector Using Deep Optical Scanning”) discloses Deep Optical Scanning (DEOS) for the detection of breast cancer and the determination of response to therapy.

U.S. Pat. No. 9,597,046 (Goossen et al., Mar. 21, 2017, “Method and Device for Imaging Soft Body Tissue Using X-Ray Projection and Optical Tomography”) discloses breast imaging using X-ray projection techniques and optical tomography techniques. U.S. patent application 20170105625 (Eum, Apr. 20, 2017, “Diagnostic Device of Optics Type for Breast”) discloses an optical breast diagnostic apparatus with a hemispherical cover. U.S. patent Ser. No. 10/200,655 (Kim et al., Feb. 5, 2019, “Tomographic Imaging Methods, Devices, and Systems”) discloses a multispectral bioluminescence optical tomography algorithm makes use of a partial differential equation (PDE) constrained approach. U.S. patent Ser. No. 10/215,636 (Fujii et al., Feb. 26, 2019, “Imaging Device Provided With Light Source That Emits Pulsed Light and Image Sensor”) discloses an imaging device with a light source that emits pulsed light at different wavelengths. U.S. patent Ser. No. 10/506,181 (Delgado et al., Dec. 10, 2019, “Device for Optical Imaging”) discloses the capture of an infrared image.

Turning to the non-patent literature, Ahmed et al., (2021), “Differential Optical Absorption Spectroscopy-Based Refractive Index Sensor for Cancer Cell Detection,” Optical Review, 28, 134-143, discloses a spectroscopic optical sensor for cancerous cell detection in various parts of the human body. Altoe et al., (2019), “Diffuse Optical Tomography of the Breast: A Potential Modifiable Biomarker of Breast Cancer Risk with Neoadjuvant Chemotherapy,” Biomedical Optics Express, Aug. 1, 2019, 10(8), 4305-4315, studied whether a diffuse optical tomography breast imaging system (DOTBIS) can provide a comparable optical-based image index of mammographic breast density. Altoe et al., (2021), “Changes in Diffuse Optical Tomography Images During Early Stages of Neoadjuvant Chemotherapy Correlate with Tumor Response in Different Breast Cancer Subtypes”, Clinical Cancer Research, Apr. 1, 2021, 27(7), 1949-1957, studied changes in optically derived parameters acquired with a diffuse optical tomography breast imaging system (DOTBIS) in the tumor volume of patients with breast carcinoma receiving neoadjuvant chemotherapy (NAC).

Altoe et al., (2021), “Effects of Neoadjuvant Chemotherapy on the Contralateral Non-Tumor-Bearing Breast Assessed by Diffuse Optical Tomography,” Breast Cancer Research, 2021, 23, 16, studied whether changes in optically derived parameters acquired with a diffuse optical tomography breast imager system (DOTBIS) in the contralateral non-tumor-bearing breast in patients administered neoadjuvant chemotherapy (NAC) for breast cancer are associated with pathologic complete response (pCR). Anabestani et al. (2022), “Advances in Flexible Organic Photodetectors: Materials and Applications,” Nanomaterials, 2022, 12(21), 3775, discusses recent advances in flexible organic photodetectors, including their applications in health-monitoring, X-ray detection, and imaging. Anderson et al., (2017), “Optical Mammography in Patients with Breast Cancer Undergoing Neoadjuvant Chemotherapy: Individual Clinical Response Index,” Academic Radiology, October, 2017, 24(10), 1240-1255, discloses an optical mammography study to develop quantitative measures of pathologic response to neoadjuvant chemotherapy (NAC) in patients with breast cancer.

Angelo et al., (2018), “Review of Structured Light in Diffuse Optical Imaging,” Journal of Biomedical Optics, Sep. 14, 2018, 24(7), 071602, discloses diffuse optical imaging probes in living tissue enabling structural, functional, metabolic, and molecular imaging. Applegate et al., (2018), “Multi-Distance Diffuse Optical Spectroscopy with a Single Optode via Hypotrochoidal Scanning,” Optics Letters, 2018, 43, 747-750, studied a new method of frequency-domain diffuse optical spectroscopy (FD-DOS) to rapidly acquire a wide range of source-detector (SD) separations by mechanically scanning a single SD pair. Applegate et al. (2020), “Recent Advances in High Speed Diffuse Optical Imaging in Biomedicine,” APL Photonics, 2020, 5(4), 040802, 21, reviews recent advances in acquisition and processing speed for several Diffuse Optical Imaging modalities.

Chae et al., (2020), “Development of Digital Breast Tomosynthesis and Diffuse Optical Tomography Fusion Imaging for Breast Cancer Detection,” Scientific Reports, 10, 13127 (2020), studied a new digital breast tomosynthesis (DBT)/DOT fusion imaging technique for breast cancer detection. Chitnis et al. (2016), “Towards a Wearable Near Infrared Spectroscopic Probe for Monitoring Concentrations of Multiple Chromophores in Biological Tissue In Vivo,” Review of Scientific Instruments, June, 2016, 87(6), 065112, discloses a wearable multi-wavelength technology for functional near-infrared spectroscopy with an 8-wavelength light emitting diode (LED) source. Cinquino et al. (2021), “Light-Emitting Textiles: Device Architectures, Working Principles, and Applications,” Micromachines, (Special Issue Emerging and Disruptive Next-Generation Technologies for POC: Sensors, Chemistry and Microfluidics for Diagnostics), 2021, 12(6), 652, discusses applications of light-emitting fabrics, including Organic LEDs.

Cochran et al., (2019), “Hybrid Time-Domain and Continuous-Wave Diffuse Optical Tomography Instrument with Concurrent, Clinical Magnetic Resonance Imaging for Breast Cancer Imaging,” Journal of Biomedical Optics, January, 2019, 24(5), 1-11, discusses diffuse optical tomography (DOT) for three-dimensional (3-D) maps of tissue optical and physiological properties in human tissue. Costa et al. (2019), “Flexible Sensors: From Materials to Applications,” Technologies (Special Issue, Reviews and Advances in Internet of Things Technologies), 2019, 7(2), 35, reviews the current state of flexible sensor technologies and the impact of material developments on this field. Durduran et al., (2010, 2010), “Diffuse Optics for Tissue Monitoring and Tomography,” Reports on Progress in Physics, 2010, 73(7), 076701, discloses using near-infrared or diffuse optical spectroscopy to measure tissue hemodynamics.

Fakayode et al., (2020), “Molecular (Raman, NIR, and FTIR) Spectroscopy and Multivariate Analysis in Consumable Products Analysis,” Applied Spectroscopy, reviews, 55:8, 647-723, reviews the use of Raman, near-infrared (NIR), and Fourier-transform infrared (FTIR) spectrometers to evaluate consumable products such as food. Fantini et al., (2001), “Optical Spectroscopy and Imaging of Tissues,” NSF Award #0093840, Jun. 1, 2001, studied development of new improved methods and instrumentation for biomedical applications of near-infrared spectroscopy and imaging. Fantini (2005), “Optical Spectroscopy and Imaging of Tissues”, NSF Award, 2005 (abstract only viewed), researched techniques for optical spectroscopy and imaging of biological tissues. Fantini et al., (2012), “Near-Infrared Optical Mammography for Breast Cancer Detection with Intrinsic Contrast,” Annals of Biomedical Engineering, February, 2012, 40(2), 398-407, reviews optical methods to detect breast cancer on the basis of increased opacity.

Farmani et al., (2020), “Optical Nanosensors for Cancer and Virus Detections,” Micro and Nano Technologies, Nanosensors for Smart Cities, Chapter 25, Han et al. editors, Elsevier, 2020, 419-432, ISBN 9780128198704, discusses photonic crystal (PhC)-based optical nanosensors. Feng et al. (2021), “MRI Guided Wearable Near Infrared Spectral Tomography: Simulation Study,” Proceedings SPIE, 11639, Optical Tomography and Spectroscopy of Tissue XIV, 116390D, Mar. 5, 2021, discloses a new low-cost imaging system for MRI-guided Near-Infrared Spectral Tomography (MRI-NIRST) for breast cancer detection. Flexman et al., (2008), “The Design and Characterization of a Digital Optical Breast Cancer Imaging System,” 30th Annual International Conference of the IEEE Engineering in Medicine and Biology Society, 2008, 3735-3738, discusses how optical imaging has the potential to play a major role in breast cancer screening and diagnosis due to its ability to image cancer characteristics such as angiogenesis and hypoxia.

Ghijsen et al., (2018), “Quantitative Real-Time Optical Imaging of the Tissue Metabolic Rate of Oxygen Consumption,” Journal of Biomedical Optics, Mar. 24, 2018, 23(3), 036013, discloses a noncontact method for quantitatively mapping tMRO2 over a wide, scalable field of view. Grosenick et al., (2016), “Review of Optical Breast Imaging and Spectroscopy,” Journal of Biomedical Optics, September, 2016, 21(9), 091311, reviews the monitoring neoadjuvant chemotherapy and breast cancer risk assessment via optical breast imaging and spectroscopy. Gunther et al., (2018), “Dynamic Diffuse Optical Tomography for Monitoring Neoadjuvant Chemotherapy in Patients with Breast Cancer,” Radiology, June, 2018; 287(3): 778-786, identifies dynamic optical imaging features associated with pathologic response in patients with breast cancer during neoadjuvant chemotherapy.

Hoi et al., (2018), “Non-Contact Dynamic Diffuse Optical Tomography Imaging System for Evaluating Lower Extremity Vasculature,” Biomedical Optics Express, 2018, 9, 5597-5614, discloses a multi-view non-contact dynamic diffuse optical tomographic imaging system for the clinical evaluation of vasculature in the lower extremities. Imamura et al., (2018), “In Vivo Optical Imaging of Cancer Cell Function and Tumor Microenvironment,” Cancer Science, 2018, 109, 912-918, discusses in vivo optical imaging using fluorescence and bioluminescence. Intes et al. (2004), “Time-Domain Optical Mammography Softscan: Initial Results On Detection and Characterization of Breast Tumors,” Proceedings SPIE 5578, Photonics North 2004: Photonic Applications in Astronomy, Biomedicine, Imaging, Materials Processing, and Education, Dec. 9, 2004 presents initial results obtained using a breast-imaging system developed by Advanced Research Technologies comprising a 4-wavelength time-resolved scanning system.

Jeong et al., (2020), “Emerging Advanced Metasurfaces: Alternatives to Conventional Bulk Optical Devices,” Microelectronic Engineering, 2020, Vol. 220, 111146, ISSN 0167-9317, discusses the use of optical metasurfaces as color filters, metalenses, beam generators or splitters, and meta-holograms. Jiang et al. (2021), “MRI-Guide Near Infrared Spectroscopic Tomographic Imaging System with Wearable Optical Breast Interface for Breast Imaging,” Proceedings SPIE, 11639, Optical Tomography and Spectroscopy of Tissue XIV, 116391J, 3/5/2021, discloses a new photo-detector (PD) and source fiber based wearable MRI-guide near infrared spectroscopic tomographic imaging (MRg-NIRST) system. Joshi et al., (2018), “Targeted Optical Imaging Agents in Cancer: Focus on Clinical Applications,” Contrast Media and Molecular Imaging, Aug. 27, 2018, discusses molecular imaging for in vivo visualization of cancer over time based on biological mechanisms of disease activity.

Jung et al. (2015), “Non-Contact Deep Tissue Imaging using a Hand-Held Near-infrared Optical Scanner,” Journal of Medical Diagnostic Methods, Mar. 24, 2015, 4(2), 1-10, discloses fiber-free non-contact near-infrared (NIR) imaging devices using wide-field detectors. Khan (2013), “Image Reconstruction in Diffuse Optical Tomography With Sparsity Constraints”, NSF Award, 2013 (abstract only viewed), researched the use of sparsity-constrained regularization for solving the diffuse optical tomography inverse problem. Kim et al., (2016), “US-Localized Diffuse Optical Tomography in Breast Cancer: Comparison with Pharmacokinetic Parameters of DCE-MRI and With Pathologic Biomarkers,” BMC Cancer, Feb. 1, 2016, 16:50, discloses correlating parameters of ultrasonography-guided diffuse optical tomography with the pharmacokinetic features of dynamic contrast-enhanced MRI and pathologic markers of breast cancer. Koetse et al., (2007), “Optical Sensor Array Platform Based on Polymer Electronic Devices,” Proceedings SPIE 6739, Electro-Optical Remote Sensing, Detection, and Photonic Technologies and Their Applications, 67391D, Nov. 7, 2007, discusses devices based on polymer semiconductors fabricated with thin film technology.

Koomson, 2019), “A Noninvasive Biological Research Tool for Measurement of Tissue and Cerebral Oxygenation,” NSF Award #1919038, Jul. 15, 2019, (abstract only viewed) investigates compact wearable devices with advanced NIRS capability. Krishnamurthy, 2018), “Using Near-Infrared Spectroscopy to Study Static and Dynamic Hemoglobin Contrast Associated with Breast Cancer,” Tufts University, Dissertation, 2018, discloses an instrument for diffuse optical mammography with parallel plate geometry. Leo et al. (2017), “Optical Imaging of the Breast: Basic Principles and Clinical Applications,” American Journal of Roentgenology, 2017, 209:1, 230-238, discloses summarizes the physical principles, technology features, and first clinical applications of optical imaging techniques to the breast. Li et al. (2018), “Sensitive and Wearable Optical Microfiber Sensor for Human Health Monitoring,” Advanced Materials Technologies, 2018, 3, 1800296, discloses a sensor with a hybrid plasmonic microfiber knot resonator embedded in a polydimethylsiloxane membrane.

Liu et al., (2018), “Diffuse Optical Spectroscopy for Monitoring the Responses of Patients with Breast Cancer to Neoadjuvant Chemotherapy: A Meta-Analysis,” Medicine, 2018, 97(41), 12683, investigated the potential of diffuse optical spectroscopy (DOT) for monitoring the responses of patients with breast cancer to neoadjuvant chemotherapy (NAC). Liu et al., (2020), “Recent Progress in Flexible Wearable Sensors for Vital Sign Monitoring,” Sensors, 2020, 20(14), 4009, discusses the development of flexible electronic materials, as well as the wide development and application of smartphones, the cloud, and wireless systems, flexible wearable sensor technology. Liu et al., (2021), “Simultaneous Measurements of Tissue Blood Flow and Oxygenation Using a Wearable Fiber-Free Optical Sensor,” Journal of Biomedical Optics, Jan. 29, 2021, 26(1), 012705, discusses a wearable dual-wavelength diffuse speckle contrast flow oximetry (DSCFO) device for simultaneous measurements of blood flow and oxygenation variation in deep tissues.

Lutzweiler et al., (2013), “Optoacoustic Imaging and Tomography: Reconstruction Approaches and Outstanding Challenges in Image Performance and Quantification,” Sensors, 2013, 13(3), 7345-7384, reviews optoacoustic imaging from image reconstruction and quantification perspectives. Ma et al. (2020b), “Fiber-Free Parallel-Plane Continuous Wave Breast Diffuse Optical Tomography System,” SPIE 11229, Advanced Biomedical and Clinical Diagnostic and Surgical Guidance Systems XVIII, Proceedings, 112290L, Feb. 21, 2020 discusses near infrared diffuse optical tomography (DOT) for detecting breast cancer. Mabou et al., (2018), “Breast Cancer Detection Using Infrared Thermal Imaging and a Deep Learning Model,” Sensors, 2018, 18(9), 2799, discloses the use of infrared digital imaging for breast cancer detection based on thermal comparison between a healthy breast and a breast with cancer.

Moreno et al. (2019), “Evaluation on Phantoms of the Feasibility of a Smart Bra to Detect Breast Cancer in Young Adults,” Sensors, 2019, 19(24), 5491, discloses the use of breast tissue phantoms to investigate the feasibility of quantifying breast density and detecting breast cancer tumors using a smart bra. Nguyen et al., (2020), “Preliminary Development of Optical Computed Tomography (Optical CT) Scanner Using Transillumination Imaging NAD,” Conference: International Symposium on Applied Science 2019, Hochiminh City, Vietnam. May 14, 2020, discusses the use of near-infrared transillumination imaging for biomedical applications such as human biometrics and animal experiments. Pan et al., (2020), “A Multifunctional Skin-Like Wearable Optical Sensor Based on an Optical Micro-/Nanofibre,” Nanoscale, 2020, Issue 33, discusses multifunctional skin-like sensors for next-generation healthcare, robotics, and bioelectronics.

Park et al., (2013), “Multispectral Imaging Using Polydimethylsiloxane (PDMS) Embedded Vertical Silicon Nanowires,” OSA Technical Digest (online) (Optical Society of America, 2013), paper CTu30.1, reports on the demonstration of a compact multispectral imaging system that uses vertical silicon nanowires for a filter array. Park et al., (2015), “Vertically Stacked Photodetector Devices Containing Silicon Nanowires with Engineered Absorption Spectra,” ACS Photonics, Mar. 16, 2015, 2(4), 544-549, discloses a vertically stacked photodetector device containing silicon nanowire photodetectors formed above a silicon substrate that also contains a photodetector. Perumal et al., (2019), “Near Infra-Red Polymeric Nanoparticle Based Optical Imaging in Cancer Diagnosis,” Journal of Photochemistry and Photobiology, Biology, 2019, Vol. 199, 111630, ISSN 1011-1344, reviews the recent progress in NIRF polymeric nanoparticles used for optical imaging particularly on cancer diagnosis.

Pinti et al. (2018), “A Review on the Use of Wearable Functional Near-Infrared Spectroscopy in Naturalistic Environments,” Japanese Psychology Research, October 2018, 60(4), 347-373, reviews the use of wearable fNIRS in naturalistic settings in the field of cognitive neuroscience. Qiu (2018), “Implantable Ultra-low Power V02 MEMS Scanner Based Surface-Enhanced Raman Spectroscope for Wide-field Tumor Imaging in Free Moving Small Animals”, NSF Award, 2018 (abstract only viewed), discloses tumor-targeting surface enhanced Raman scattering nanoparticles based on multiplexed Raman spectroscopy. Rahman et al., (2016), “Electromagnetic Performances Analysis of an Ultra-Wideband and Flexible Material Antenna in Microwave Breast Imaging: To Implement a Wearable Medical Bra,” Scientific Reports, 2016, Vol. 6, 38906, discloses a compact and ultra-wide band antenna on a flexible substrate for microwave imaging.

Ray et al. (2017), “A Systematic Review of Wearable Systems for Cancer Detection: Current State and Challenges,” Journal of Medical Systems, Oct. 2, 2017, 41(11), 180, reviews cancer detection using wearable systems, including sensor-based smart systems with a microcontroller, Bluetooth module, and smart phone. Robbins et al. (2021), “Two-Layer Spatial Frequency Domain Imaging of Compression-Induced Hemodynamic Changes in Breast Tissue,” Journal of Biomedical Optics, May 24, 2021, 26(5), 056005, studied hemodynamic changes in response to localized breast compression using a handheld SFDI device. Roblyer et al. (2020b), “Tracking Breast Cancer Therapies with Handheld and Wearable Diffuse Optics,” Biophotonics Congress: Biomedical Optics 2020 (Translational, Microscopy, OCT, OTS, BRAIN), OSA Technical Digest (Optical Society of America, 2020), paper TM4B.1 disclose an NIR-II imaging system), “Detection of Optically Luminescent Probes using Hyperspectral and Diffuse Imaging in Near-infrared” (DOLPHIN) for noninvasive real-time tracking of a 0.1 mm-sized fluorophore through the gastrointestinal tract of a mouse.

Saikia et al. (2017), “A Cost-Effective LED and Photodetector Based Fast Direct 3D Diffuse Optical Imaging System,” Proc. SPIE 10412, Diffuse Optical Spectroscopy and Imaging VI, Jul. 28, 2017, European Conferences on Biomedical Optics, 2017, Munich, Germany, discloses a cost-effective and high-speed 3D diffuse optical tomography system using high power LED light sources and silicon photodetectors. Saikia et al. (2019), “A Point-of-Care Handheld Region-of-Interest (ROI) 3D Functional Diffuse Optical Tomography (fDOT) System,” Proc. SPIE 10874, Optical Tomography and Spectroscopy of Tissue XIII, Mar. 1, 2019, discloses a 3D Functional Diffuse Optical Tomography (fDOT) system based on an Internet-of-things (IoT) concept. Satharasinghe et al. (2018), “Photodiodes Embedded Within Electronic Textiles,” Science Reports, 2018, 8, 16205, discloses a novel photodiode-embedded yarn with possible applications including monitoring body vital signs.

Schoustra et al. (2021), “Pendant Breast Immobilization and Positioning in Photoacoustic Tomographic Imaging,” Photoacoustics, 2021, 21, 100238 describes the design, development and added value of breast-supporting cups to immobilize and position the pendant breast in photoacoustic tomographic imaging. Shokoufi et al. (2017), “Novel Handheld Diffuse Optical Spectroscopy Probe for Breast Cancer Assessment: Clinical Study,” Journal of Biomedical Science, 6(5), 34, discloses a hand-held continuous-wave radio-frequency modulated diffuse optical spectroscopy probe. Soliman et al., (2010), “Functional Imaging Using Diffuse Optical Spectroscopy of Neoadjuvant Chemotherapy Response in Women with Locally Advanced Breast Cancer,” Clinical Cancer Research, Apr. 20, 2010, 15, 2605-2614, discloses functional imaging with tomographic near-infrared diffuse optical spectroscopy to measure tissue concentration of deoxyhemoglobin, oxyhemoglobin, percent water, and scattering power.

Spink et al. (2020), “High Optode-Density Wearable Probe for Monitoring Breast Tumor Dynamics During Neoadjuvant Chemotherapy,” Biophotonics Congress: Biomedical Optics 2020 (Translational, Microscopy, OCT, OTS, BRAIN), OSA Technical Digest (Optical Society of America, 2020), paper TTu1B.2 disclose an NIR-II imaging system), “Detection of Optically Luminescent Probes using Hyperspectral and diffuse Imaging in Near-infrared” (DOLPHIN). Spink et al. (2021), “High Optode-Density Wearable Diffuse Optical Probe for Monitoring Paced Breathing Hemodynamics in Breast Tissue,” Journal of Biomedical Optics, Jun. 2, 2021, 26(6), 062708, discloses a high optode-density wearable continuous wave diffuse optical probe for the monitoring of breathing hemodynamics in breast tissue. Tank et al. (2020), “Diffuse Optical Spectroscopic Imaging Reveals Distinct Early Breast Tumor Hemodynamic Responses to Metronomic and Maximum Tolerated Dose Regimens,” Breast Cancer Research, 2020, 22, 29 reports on a dual-center study which examined 54 breast tumors receiving NAC measured with DOSI before therapy and the first week following chemotherapy administration.

Teng (2018), “A Wearable Near-Infrared Diffuse Optical System for Monitoring in Vivo Breast Tumor Hemodynamics During Chemotherapy Infusions,” Boston University, Dissertation, 2018, discloses a new wearable diffuse optical device to investigate if very early timepoints during a patient's first chemotherapy infusion are predictive of overall response (pCR versus non-pCR) to NAC. Teng et al. (2017), “Wearable Near-Infrared Optical Probe for Continuous Monitoring During Breast Cancer Neoadjuvant Chemotherapy Infusions,” Journal of Biomedical Optics, 22(1), 14001 presents a new continuous-wave wearable diffuse optical probe for investigating the hemodynamic response of locally advanced breast cancer patients during neoadjuvant chemotherapy infusions. Tiwari et al. (2022), “Role of Sensor Technology in Detection of the Breast Cancer,” BioNanoScience, 2022, 12, 639-659, reviews different sensors developed to detect breast cancer over the past few years.

Tromberg et al., (2016), “ACRIN 6691 Investigators. Predicting Responses to Neoadjuvant Chemotherapy in Breast Cancer,” Cancer Research, Aug. 15, 2016, 76(20), 5933-5944, investigates whether changes from baseline to mid-therapy in a diffuse optical spectroscopic imaging (DOSI)-derived imaging endpoint, the tissue optical index, predict pathologic complete response in women undergoing breast cancer neoadjuvant chemotherapy. Uddin et al., (2020a), “Optimal Breast Cancer Diagnostic Strategy Using Combined Ultrasound and Diffuse Optical Tomography,” Biomedical Optics Express, 11(5), 2722-2737, presents a two-stage diagnostic strategy that is both computationally efficient and accurate. Upputuri, (2019), “Photoacoustic Imaging in the Second Near-Infrared Window: A Review,” Journal of Biomedical Optics, Apr. 9, 2019, 24(4), 040901, discusses photoacoustic (PA) imaging that combines optical excitation and ultrasound detection.

Vavadi et al., (2018), “Compact Ultrasound-Guided Diffuse Optical Tomography System for Breast Cancer Imaging,” Journal of Biomedical Optics, 2018, 24(2), 1-9, discusses an ultrasound-guided DOT system. Wang et al. (2020), “Development of a Prototype of a Wearable Flexible Electro-Optical Imaging System for the Breast,” Biophotonics Congress: Biomedical Optics 2020 (Translational, Microscopy, OCT, OTS, BRAIN), OSA Technical Digest (Optical Society of America, 2020), paper TM4B.4, discloses a wearable breast imaging system which combines a garment and a flexible electronic system. Yu et al. (2010), “Near-Infrared, Broad-Band Spectral Imaging of the Human Breast for Quantitative Oximetry: Applications to Healthy and Cancerous Breasts,” Journal of Innovative Optical Health Sciences, October 2010, 03(4):267-277 discusses the examination of ten human subjects with a previously developed instrument for near-infrared diffuse spectral imaging of the female breast.

Yuan et al. (2014), “Light-Emitting Diode-Based Multiwavelength Diffuse Optical Tomography System Guided by Ultrasound,” Journal of Biomedical Optics, Dec. 4, 2014, 19(12) 126003, discloses a low-cost DOT system using LEDs of four wavelengths in the NIR spectrum as light sources. Zhang et al., (2020), “Efficacy of Shear-Wave Elastography Versus Dynamic Optical Breast Imaging for Predicting the Pathological Response to Neoadjuvant Chemotherapy in Breast Cancer,” European Journal of Radiology, 2020, 129, 109098, discusses the value of shear-wave elastography (SWE) parameters and dynamic optical breast imaging features for predicting pathological responses to neoadjuvant chemotherapy (NACT) in breast cancer (BC).

Zhao et al. (2021), “High Resolution, Deep Imaging Using Confocal Time-of-Flight Diffuse Optical Tomography,” IEEE Transactions on Pattern Analysis and Machine Intelligence, 7/1/2021, 43(7), 2206-2219, discloses how time-of-flight diffuse optical tomography (ToF-DOT) can achieve millimeter spatial resolution in the highly scattered diffusion regime. Zhao et al. (2022), “MRI-Guided Near-Infrared Spectroscopic Tomography (MRg-NIRST): System Development for Wearable, Simultaneous NIRS and MRI Imaging,” Proc. SPIE 11952, Multimodal Biomedical Imaging XVII, Mar. 2, 2022, discloses a novel wearable MRg-NIRST system for breast cancer detection with eight flex circuit strips, each with six photo-detectors (PDs) and six source fibers.

Zhu et al. (2020), “A Review of Optical Breast Imaging: Multi-Modality Systems for Breast Cancer Diagnosis,” European Journal of Radiology, August 2020, 129:109067, reviews optical breast imaging using multi-modality platforms. Zhu et al., (2021), “Early Assessment Window for Predicting Breast Cancer Neoadjuvant Therapy Using Biomarkers, Ultrasound, and Diffuse Optical Tomography,” Breast Cancer Research and Treatment, 2021, assesses the utility of tumor biomarkers, ultrasound (US) and US-guided diffuse optical tomography (DOT) in early prediction of breast cancer response to neoadjuvant therapy (NAT).

### SUMMARY OF THE INVENTION

Disclosed herein is a smart bra for detection of abnormal breast tissue using a plurality of optical modules (including light emitters and light receivers) on at least one cup of the bra. Light from the light emitters is received by the light receivers after it has been transmitted through breast tissue. This light is then analyzed to detect, locate, image, and/or characterize abnormal breast tissue.

In an example, an optical module can have two light emitters and a light receiver, wherein the two light emitters emit light with different wavelengths. In an example, an optical module can also include a movable light guide (such as a movable micromirror) which reflects a beam of light from a light emitter toward a breast. In an example, the smart bra can further comprise expandable chambers or piezoelectric bands which gently compress the breast to improve optical scanning.

### INTRODUCTION TO THE FIGURES

FIG. 1 shows a smart bra and a close-up of a cup with optical modules around its perimeter.

FIGS. 2 through 5 shows four sequential views of a cup with optical modules (each having two light emitters, a movable light guide, and a light receiver) as the optical modules scan breast tissue from different angles.

FIG. 6 shows a smart bra and a close-up a cup with nested rings of optical modules.

FIG. 7 shows a smart bra and a close-up of a cup with optical modules and light receivers around its perimeter.

FIG. 8 shows a cup wherein: the upper-outer quadrant of its perimeter has an outward-facing concavity; and the upper-inner quadrant of its perimeter has a first section with an outward-facing concavity and a second section with an inward-facing concavity.

FIG. 9 shows a cup wherein: the upper-outer quadrant of its perimeter has an inward-facing concavity; and the upper-inner quadrant of its perimeter has a first section with an outward-facing concavity and a second section with an inward-facing concavity.

FIG. 10 shows a cup wherein: the upper-outer quadrant of its perimeter has an inward-facing concavity; and the upper-inner quadrant of its perimeter has an outward-facing concavity.

FIG. 11 shows a cup wherein: the upper-outer quadrant of its perimeter has an inward-facing concavity; and the upper-inner quadrant of its perimeter has an inward-facing concavity.

FIG. 12 shows a cup with a ring of optical modules each having two light emitters, a movable light guide, and a light receiver.

FIG. 13 shows a cup with a ring of optical modules each having a light emitter, a movable light guide, and a light receiver.

FIG. 14 shows a cup with a ring of optical modules, wherein some modules each have a light emitter and a movable light guide, and wherein some modules each have a light receiver.

FIG. 15 shows a cup with a ring of optical modules, wherein modules on one side each have a light emitter and a movable light guide, and wherein modules on the opposite side each have a light receiver.

FIGS. 16 and 17 show two sequential views of an optical module with a light emitter and a movable light guide wherein the light guide moves and reflects light from the light emitter toward a breast at different angles.

FIG. 18 shows frontal and lateral views of a cup with nested rings of optical modules.

FIG. 19 shows frontal and lateral views of a cup a radial array of optical modules.

FIG. 20 shows two sequential views of a cup with nested rings of optical modules scanning breast tissue at different angles.

FIG. 21 shows two sequential views of a cup with a radial array of optical modules scanning breast tissue at different angles.

FIG. 22 shows two sequential views of a cup with optical modules and piezoelectric bands whose contraction compresses a breast.

FIG. 23 shows two sequential views of a cup with optical modules and expandable chambers whose expansion compresses a breast.

FIG. 24 shows: a lateral view of a cup with optical modules and electroconductive pathways; and a close-up view of an optical module with a light emitter, a movable light guide, and a light receiver.

FIG. 25 shows: a lateral view of a cup with optical modules, electroconductive pathways, and piezoelectric bands; and a close-up view of an optical module with a light emitter, a movable light guide, and a light receiver.

FIG. 26 shows a lateral view of a cup with nested rings of light emitters, light receivers, and electroconductive pathways.

FIG. 27 shows a lateral view of a cup with nested rings of light emitters, light receivers, and electroconductive pathways, wherein light emitters are on one side of the cup and light receivers are on the opposite side of the cup.

FIG. 28 shows a cup with a repeating sequence of two light emitters and a light receiver around its perimeter.

FIG. 29 shows a cup with a repeating sequence of two light emitters and a light receiver around its perimeter, wherein the two light emitters emit light with different wavelengths.

FIGS. 30 and 31 shows two examples of a cup with light emitters on one side of its perimeter and light receivers on the opposite side of its perimeter.

FIGS. 32 and 33 show two sequential frontal views of a cup with expandable chambers which expand into convex lens or football shapes.

FIGS. 34 and 35 show two sequential frontal views of a cup with expandable chambers which expand into crescent or banana shapes.

FIG. 36 shows two sequential lateral views of cup with a relatively-inelastic proximal portion, a relatively-elastic distal portion, expandable chambers, and optical modules.

FIG. 37 shows two sequential front views of smart bra whose cups have optical modules, expandable chambers, and relatively-inelastic bands or layers between the optical modules and the expandable chambers.

### DETAILED DISCUSSION OF THE FIGURES

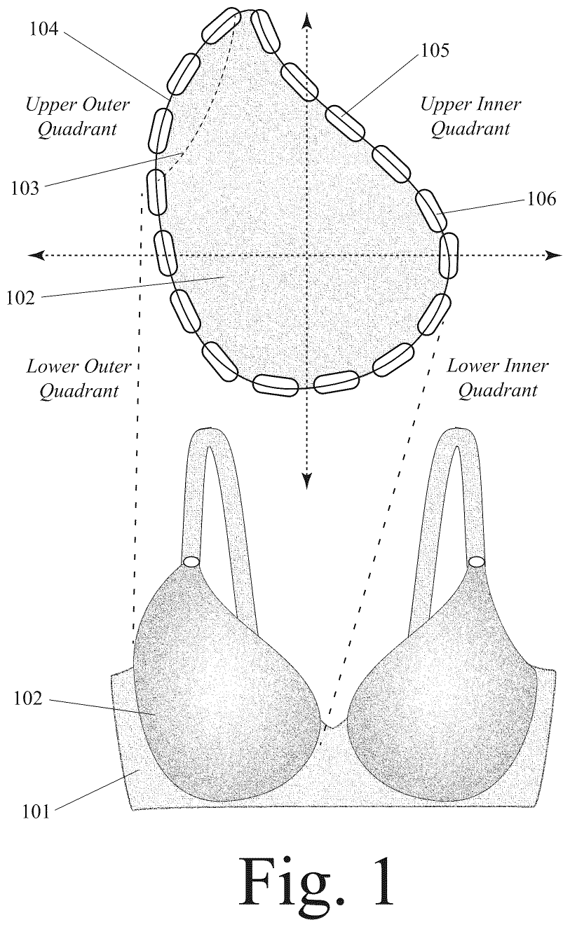

Before discussing the specific embodiments of this invention which are shown in FIGS. 1 through 37, this disclosure provides an introductory section which covers some of the general concepts, components, and methods which comprise this invention. Where relevant, these concepts, components, and methods can be applied as variations to the examples shown in FIGS. 1 through 37 which are discussed afterwards.

In an example, a smart bra for optical detection of abnormal breast tissue can comprise: a bra with cups which are configured to hold a person's breasts; wherein at least one of the cups has a plurality of optical modules; wherein the plurality of optical modules further comprises light emitters and light receivers; wherein light from the light emitters is received by the light receivers after the light has been transmitted through and/or reflected by breast tissue; and wherein light received by the light receivers is analyzed to detect, locate, image, and/or characterize abnormal breast tissue.

In an example, a plurality of optical modules can be arranged in rings. In an example, the rings can be nested from a frontal perspective. In an example, a plurality of optical modules can be arranged in a radial array. In an example, a radial array can be a starburst array. In an example, a radial array can be a hub-and-spoke array. In an example, a plurality of optical modules can be arranged in a honeycomb grid.

In an example, optical modules in a plurality of optical modules can each have both a light emitter and a light receiver. In an example, optical modules in a plurality of optical modules can each have two light emitters and a light receiver. In an example, two light emitters can emit light with different wavelengths. In an example, two light emitters can emit light at different times. In an example, a first set of optical modules in a plurality of optical modules can each have a light emitter but no light receiver and a second set of optical modules in the plurality of optical modules can each have a light receiver but no light emitter.

In an example, each optical module in a plurality of optical modules can have a light emitter and a movable light guide. In an example, a movable light guide can reflect and/or refract a beam of light from a light emitter toward a breast. In an example, a light guide can be a movable mirror. In an example, each optical module in a plurality of optical modules can have a light emitter, a movable light guide, and a light receiver. In an example, each optical module in a plurality of optical modules can have a first light emitter, a second light emitter, a movable light guide, and a light receiver. In an example, a movable light guide can be located between a first light emitter and a second light emitter.

In an example, a smart bra can further comprise a plurality of expandable chambers, wherein expansion of the expandable chambers compresses a breast. In an example, a smart bra can further comprise a plurality of piezoelectric contracting bands or layers, wherein contraction of the piezoelectric contracting bands or layers compresses a breast.

In an example, a smart bra for optical detection of abnormal breast tissue (e.g. breast cancer) can come in different sizes corresponding to conventional smart bra sizes. In an example, a left-side version of a cup on the smart bra can be symmetric to a right-side version of the cup. In an example, the results from optically-scanning right and left breasts can be compared and/or contrasted to each other to help detect abnormal breast tissue. In this disclosure, proximal means closer to a person's chest wall and distal means farther from the person's chest wall when a smart bra is being worn by the person.

In another example, a light emitter can be a Light Emitting Diode (LED). In an example, a light emitter can be a laser LED. In an example, a light emitter can be a MicroLED. In an example, a light emitter can be a Multi-wavelength Light Emitting Diode (MWLED). In an example, a light emitter can be a pulsatile laser. In another example, a light emitter can be a Resonant Cavity Light Emitting Diode (RCLED). In an example, a light emitter can be a Single Photon Avalanche Diode (SPAD). In an example, a light emitter can be a Super-Luminescent Light Emitting Diode (SLED). In an example, a light emitter can be an Active Matrix Organic Light-Emitting Diode (AMOLED). In an example, a light emitter can be an Organic Light Emitting Diode (OLED). In an example, a light emitter can be a Light-Emitting Electrochemical Cells (LECs). In another example, a light emitter can emit coherent light. In an example, a light emitter can emit light pulses. In another example, a light emitter can transmit light into and/or onto breast tissue.

In an example, light emitters can be configured along undulating (e.g. sinusoidal) pathways. In an example, light emitters can be configured in a spiral array. In an example, a smart bra cup can comprise a radial array (e.g. starburst or hub-and-spoke array) of optical modules. In an example, a smart bra cup can further comprise a helical (e.g. spiral) grid of light emitters and light receivers. In an example, a smart bra cup can further comprise a honeycomb-shaped array of light emitters and light receivers. In an example, an array of optical modules can be a radial array, wherein spokes of the array along which optical modules are located extend out radially from an apex of the smart bra cup. In another example, light emitters can be configured in a honeycomb array (e.g. hexagonal grid or mesh). In an example, optical modules on a smart bra cup can be arranged in a radial, hub-and-spoke, or star-burst array, wherein some rays (or spokes) are shorter than others. In another example, optical modules on a smart bra cup can be arranged in a radial, hub-and-spoke, or star-burst array comprising four longer rays (or spokes) and eight shorter rays (or spokes).

In an example, a smart bra cup can have rings of optical modules. In an example, a smart bra cup can have rings of optical modules, wherein the rings have (up-side-down) comma or apostrophe shapes from a frontal perspective. In an example, a smart bra cup can have rings of optical modules which are nested when viewed from a transparent (e.g. two-dimensional) frontal perspective. In an example, a smart bra cup can have rings of optical modules, wherein the rings are nested from a transparent (e.g. two dimensional) perspective, and wherein an outer ring and a middle ring are closer together than the middle ring and an inner ring.

In an example, a smart bra cup can have rings of optical modules, wherein the rings are nested from a transparent (e.g. two dimensional) perspective, and wherein inner rings are closer together than outer rings. In an example, an array of optical modules can comprise an array of nested (e.g. concentric and/or coaxial) rings, wherein optical modules are located around the rings. In another example, optical modules in an array of (three) nested rings can be equally-distributed (e.g. evenly spaced) in all of (three) the rings.

In an example, a cross-section of a cup can have a paisley shape. In another example, a perimeter of a cross-section of a cup can have an elliptical or oval shape. In an example, a perimeter of a smart bra cup can have a comma, teardrop, or paisley shape. In an example, a proximal portion of a smart bra cup can have a frustal and/or partial-conical shape. In an example, a smart bra cup can have a conical, partial-conical, and/or frustal shape. In an example, a smart bra cup can have a teardrop shape with a vertex or point, wherein the vertex or point is configured to be placed over the Tail of Spence. In an example, a smart bra cup can have an elliptical, oval, and/or oblate-circular shape.

In an example, a smart bra cup can comprise a plurality of optical modules around the perimeter of the cup. In an example, light emitters and receivers in a smart bra cup can span between 60% and 85% of the interior concavity of the cup. In another example, light emitters and receivers in a smart bra cup can span between 50% and 75% of the perimeter of the cup. In an example, optical modules can be distributed around a perimeter of a smart bra cup. In another example, optical modules in a smart bra cup can span between 50% and 75% of the interior concavity of the cup. In an example, optical modules in a smart bra cup can span between 60% and 85% of the perimeter of the cup.

In an example, a first light emitter can emit intensity or amplitude modulated light into the breast with a wavelength in the range of 650 to 750 nm; a second light emitter can emit intensity or amplitude modulated light with a wavelength in the range of 750 nm to 850 nm; and a third light emitter can emit intensity or amplitude modulated light with a wavelength in the range of 850 nm to 950 nm. In an example, a first light emitter can emit light with a wavelength in the range of 650 to 750 nm; a second light emitter can emit light with a wavelength in the range of 750 nm to 850 nm; and a third light emitter can emit light with a wavelength in the range of 850 nm to 950 nm.

In an example, a first light emitter can emit light with a wavelength in the range of 650 to 700 nm; a second light emitter can emit light with a wavelength in the range of 700 nm to 750 nm; and a third light emitter can emit light with a wavelength in the range of 750 nm to 800 nm. In an example, a first light emitter can emit light with a wavelength in the range of 600 to 900 nm at a first time; a second light emitter can emit light with a wavelength in the range of 900 nm to 1200 nm at a second time; and a third light emitter can emit light with a wavelength in the range of 1200 nm to 1500 nm at a third time. In an example, a first light emitter can emit light with a wavelength in the range of 600 to 800 nm at a first time; a second light emitter can emit light with a wavelength in the range of 800 nm to 1000 nm at a second time; and a third light emitter can emit light with a wavelength in the range of 1000 nm to 1200 nm at a third time.

In an example, a first light emitter can emit near-infrared light and a second light emitter can emit visible light. In another example, a first set of light emitters can emit light at a first frequency and/or wavelength (or in a first spectral range), a second set of light emitters can emit light at a second frequency and/or wavelength (or in a second spectral range), and a third set of light emitters can emit light at a third frequency and/or wavelength (or in a third spectral range). In an example, a light emitter can emit light at a frequency and/or wavelength which varies over time. In another example, a light emitter can emit light at different wavelengths over time, within the range of 600 to 1100 nm. In an example, a light emitter can emit light with a variable frequency.

In another example, an optical module can have multiple light emitters which emit light at different wavelengths. In an example, different light emitters can emit light with different wavelengths selected from the group consisting of: 600, 650, 660, 680, 690, 750, 775, 780, 785, 800, 808, 810, 830, 850, and 1000 nm. In an example, light emitters in a smart bra cup can emit light at different wavelengths over time, within the range of 600 to 1100 nm. In an example, one or more light emitters in a smart bra cup can be infrared light emitters. In an example, the wavelengths and/or frequencies of light from light emitters can be shifted up and down in a repeated pattern. In an example, the wavelengths and/or frequencies of light from light emitters can be changed in a sequential manner.

In an example, a first light emitter can emit a pulse of light with a first duration and a second light emitter can emit a pulse of light with a second duration, wherein the second duration is greater than the first duration. In an example, different optical modules can be activated to transmit light into a breast at different times. In an example, optical modules in an array of optical modules can be activated in a sequential manner.

In another example, a light receiver can be a photodetector which absorbs photons and generates electrical current, thereby converting light to electricity. In an example, a light receiver can be a thin-film photoreceptor. In another example, a light receiver can be selected from the group consisting of: photodiode, photomultiplier, photoconductor, avalanche photodiode, organic photodiode, organic photo-detector, silicon photodiode, photon multiplier, polarization-sensitive photodetector, Charge-Coupled Device (CCD), and silicon Photo-Multiplier (SiPM). In an example, a light receiver can be a photodiode.

In an example, a light receiver can receive light from a light emitter which is located in the same optical module as the light receiver. In an example, a light receiver can receive light from a light emitter after this light has been transmitted through (and/or been reflected by) breast tissue. In an example, a smart bra cup can further comprise optical shielding and/or light barriers between light emitters and light receivers to reduce the direct transmission of light from light emitters to light receivers without having been reflected by, or transmitted through, breast tissue. In an example, an optical module can include opaque shielding and/or light barriers between light emitters and light receivers to reduce the direct transmission of light from light emitters to light receivers without having been reflected by, or transmitted through, breast tissue.

In an example, a light guide can be a moving (e.g. pivoting or rotating) lens. In another example, a light guide can refract light from a light emitter. In an example, a movable light guide can reflect and/or refract light from a first light emitter toward a breast when the light guide is in a first configuration and can reflect and/or refract light from a second light emitter toward the breast when the light guide is in a second configuration. In another example, a movable light guide in an optical module in a smart bra cup can continuously oscillate (e.g. pivot or rotate) back-and-forth, thereby causing a beam of light from a light emitter to scan back and forth through a section of breast tissue. In an example, a movable light guide in an optical module in a smart bra cup can move (e.g. pivot and/or rotate) with a continuous back-and-forth (e.g. sinusoidal) movement. In an example, an optical module can comprise a light receiver, a movable light guide, a first light emitter to one side of the light guide, and a second light emitter to the other side of the light guide.

In an example, an optical module can further comprise a light guide. In an example, an optical module can further comprise a movable refractive light guide. In an example, light from a first light emitter can reflected by a first side of a light guide toward a breast at a first time when the light guide is pivoted and/or rotated in a first direction and light from a second light emitter can be reflected by a second (e.g. opposite) side of the light guide toward the breast at a second time when the light guide is pivoted and/or rotated in a second direction. In an example, light from a light emitter can be reflected by a light guide toward a breast. In an example, movement (e.g. pivoting and/or rotation) of a light guide can change the vector along which light from the optical module is transmitted into (and/or onto) a breast.

In an example, a light guide can be a micromirror array. In another example, a light guide can be a moving (e.g. pivoting or rotating) mirror (e.g. micromirror). In an example, a movable light guide can be a digital micromirror device. In another example, a movable light guide can be a pivoting and/or rotating (micro) mirror. In an example, a light guide can be a moving (e.g. pivoting or rotating) prism which is moved by interaction with a changing electromagnetic field. In another example, a light guide can be a moving (e.g. pivoting or rotating) lens which is moved by interaction with a changing electromagnetic field. In an example, an optical module can further comprise a movable light guide which is moved by interaction with a changing electromagnetic field. In an example, an optical module can further comprise two electrodes, wherein transmission of electrical energy through the electrodes creates an electromagnetic field which moves (e.g. pivots or rotates) a light guide, thereby changing the angle at which light from a light emitter is reflected by the light guide into a breast.

In an example, a movable light guide can reflect and/or refract light from the light emitter at different angles (or along different vectors) toward the breast held by the cup as the light guide pivots and/or rotates. In an example, a movable light guide in an optical module in a smart bra cup can move (e.g. pivot and/or rotate) in a continuous manner, thereby causing a beam of light from a light emitter to smoothly sweep (e.g. scan) through breast tissue at different angles (e.g. along different vectors). In an example, angles between the focal vectors of light beams emitted from optical modules and the surface of a breast or cup can vary with the distance of the modules from the apex of a cup. In an example, data concerning transmission of light beams through breast tissue along multiple vectors in multiple cross-sections of the breast can be compiled to create a three-dimensional model (e.g. three-dimensional image) of the breast.

In an example, different light emitter and light receiver pairs can be activated at different times to scan breast tissue along different vectors. In an example, light beams can be transmitted through breast tissue along multiple vectors between pairs of light emitters and light receivers at different locations around the perimeter of a cup, wherein data concerning transmission of these light beams can be compiled and analyzed to image, detect, locate, and/or characterize abnormal tissue in the breast. In an example, light beams can be transmitted through breast tissue along multiple vectors between multiple pairs of optical components, wherein data concerning transmission of these light beams can be compiled to create a two-dimensional cross-sectional images and/or a three-dimensional image of the breast.

In an example, movement (e.g. pivoting and/or rotation) of a light guide can cause beams of light from a light emitter to scan a two-dimensional cross-section of a breast at different angles. In an example, optical modules in a ring can be sequentially-activated (e.g. in a clockwise sequence around the ring) to transmit pulses of light into a breast along different vectors at different times. In an example, the angles at which an array of optical modules (e.g. including light emitters) direct light into breast tissue can be changed (e.g. controlled) by an array of electromagnetic actuators.

In another example, an optical module can comprise two light emitters and a movable light guide (e.g. mirror), wherein the light guide is between the two light emitters. In an example, an optical module can have multiple light emitters and one light receiver. In another example, an optical module can have two light emitters and one light receiver. In an example, an optical module can include one light emitter and at least four light receivers which are evenly-distributed around the light emitter. In another example, an optical module can include one light emitter and a plurality of light receivers distributed around the light emitter. In an example, an optical module can include one light receiver and a plurality of light emitters which are evenly-distributed around the light receiver. In an example, some optical modules can include light emitters and some optical modules can include light receivers.

In an example, a smart bra cup can further comprise a repeating (e.g. e1-e2-r) sequence of light emitters and light receivers around (a portion of) the perimeter of the cup, wherein (e1) is a near-infrared light emitter, (e2) is a red light emitter, and (r) is a light receiver. In an example, a smart bra cup can further comprise a repeating (e.g. e-r) sequence of light emitters and light receivers around (a portion of) the perimeter of the cup, wherein (e) is a light emitter and (r) is a light receiver. In an example, a smart bra cup can further comprise an alternating sequence of light emitters and light receivers around the perimeters of a plurality of (vertical plane) cross-sections of the cup. In an example, one light emitter in a repeating sequence of light emitters can emit visible light. In an example, one light emitter in a repeating sequence of light emitters can emit green visible light. In an example, there can be a repeating sequence of two light emitters and a light detector around a perimeter of a smart bra cup.

In another example, a first expandable chamber in a smart bra cup can be expanded to a different size than a second expandable chamber in the smart bra cup. In an example, a plurality of expandable chambers can be individually and selectively expanded so that they compress wider portions of the breast to a greater degree than narrower portions of the breast, enabling better (e.g. more uniform) transmission of light through the breast. In another example, a plurality of expandable chambers on either side (e.g. right and left) of a cup can be selectively, independently, and differentially expanded to gently compress a breast held by the cup.

In an example, a side and/or surface of an expandable chamber which faces toward the breast can have a lower Shore value than the side and/or surface of the component which faces away from the breast. In another example, a smart bra cup can comprise a first expandable chamber in the upper-outer quadrant of a cup and a second expandable chamber in the lower-inner quadrant of the cup. In an example, an expandable chamber can be expanded by a pneumatic mechanism. In an example, expansion of a plurality of expandable chambers in a smart bra cup can be controlled by a handheld push button or lever, wherein expansion stops immediately (or perhaps even reverses a little) when the button or lever is released.

In an example, a plurality of expandable chambers can be individually and selectively inflated so that they compress wider portions of the breast to a greater degree than narrower portions of the breast, enabling better (e.g. more uniform) transmission of light through the breast. In an example, a smart bra cup can comprise: a plurality of expandable chambers; a plurality of movable rigid (e.g. low-elasticity) sections (e.g. panels, bands, or strips); and a plurality of optical modules (e.g. further comprising light emitters, light receivers, or both); wherein the movable rigid sections are between the expandable chambers and the optical modules, wherein the optical modules are between the movable rigid sections and the breast held by the cup, and wherein filling the expandable chambers with a flowable substance (e.g. a gas, liquid, or gel) expands the chambers and pushes the movable rigid sections toward the breast to gently compress the breast.

In an example, a smart bra cup can comprise: a plurality of light emitters in the upper-outer and upper-inner quadrants of the cup; a plurality of light receivers in the lower-outer and lower-inner quadrants of the cup; and an expandable chamber in the lower-outer and lower-inner quadrants of the cup, wherein filling this expandable chamber with a flowable substance (e.g. a gas, liquid, or gel) gently lifts, compresses, and flattens a breast held by the cup to decrease the distances between the light emitters and light receivers. In an example, a smart bra cup can comprise: a plurality of light emitters in the lower-inner quadrant of the cup; a plurality of light receivers in the upper-outer quadrant of the cup; a first expandable chamber in the upper-outer quadrant of the cup; and a second expandable chamber in the lower-inner quadrant of the cup, wherein filling these expandable chambers with a flowable substance (e.g. a gas, liquid, or gel) gently compresses a breast held by the cup to decrease the distances between the light emitters and light receivers.

In another example, a smart bra cup can further comprise expandable chambers on diagonally-opposite quadrants (e.g. upper-outer and lower-inner quadrants) of the cup which are expanded by being filled with a flowable substance (e.g. air or a liquid), wherein expansion of these one or more chambers gently compresses the breast for improved optical scanning. In an example, a smart bra cup can further comprise one or more expandable chambers in the upper-outer quadrant of the cup which are expanded by being filled with a flowable substance (e.g. air or a liquid), wherein expansion of these one or more chambers gently compresses the breast for improved optical scanning. In another example, a smart bra cup can further comprise one or more expandable chambers which are expanded by being filled with a flowable substance (e.g. air or a liquid).

In an example, a smart bra cup can comprise two expandable chambers and a plurality of optical modules between the expandable chambers and a breast held by the cup, wherein the expandable chambers have convex lens and/or football shapes when they are expanded. In another example, an expandable chamber can be shaped like a half of a circle, ring, or torus. In an example, an expandable chamber can have a conic-section shape. In an example, an expandable chamber can have a shape which is selected from the group consisting of: pancake, disk, ellipsoidal, oblong, oval, toroidal, hemispherical, and spherical. In an example, there can be different numbers, sizes, shapes, and/or elasticities of expandable chambers in different quadrants of a cup. In an example, a smart bra can include a plurality of expandable chambers, a pump which pumps a flowable substance (e.g. a gas, liquid, or gel) into the chambers, and a plurality of tubes which conduct the flowable substance from the pump to the chambers.

In an example, a smart bra cup can further comprise a plurality of piezoelectric bands with inward-facing concavities, wherein the piezoelectric bands which contract when electrical energy is applied to them. In an example, a smart bra cup can further comprise a plurality of piezoelectric bands which contract when electrical energy is applied to them, wherein a first subset of the piezoelectric bands is in the outer (e.g. left or right) half of the cup and a second subset of the piezoelectric bands is on the inner (e.g. right or left) half of the cup. In an example, a smart bra cup can further comprise one or more piezoelectric bands on diagonally-opposite quadrants (e.g. upper-inner and lower-outer quadrants) of the cup which are contracted by application of electromagnetic energy, wherein expansion of these one or more piezoelectric bands gently compresses the breast for improved optical scanning.

In an example, a smart bra cup can further comprise one or more piezoelectric bands in the lower-inner quadrant of the cup which are contracted by application of electromagnetic energy, wherein expansion of these one or more piezoelectric bands gently compresses the breast for improved optical scanning. In another example, a smart bra cup can further comprise one or more piezoelectric bands on opposite sides (e.g. right and left sides) of the cup which are contracted by application of electromagnetic energy, wherein expansion of these one or more piezoelectric bands gently compresses the breast for improved optical scanning.

In an example, a smart bra cup can further comprise a flexible, elastic, and/or stretchable hexagonal (e.g. honeycomb) grid of electroconductive pathways which connect light emitters and light receivers to a power source. In another example, a smart bra cup can further comprise a hexagonal (e.g. honeycomb) grid of electroconductive pathways which connect light emitters and light receivers to a power source. In an example, a smart bra cup can further comprise a plurality of electroconductive pathways which provide electrical power to optical modules and transmit data from the optical modules to a data processor. In another example, a smart bra cup can have a plurality of elastic electroconductive pathways which are configured in a radial (e.g. hub-and-spoke or starburst) pattern.

In an example, a smart bra cup can have electroconductive pathways which are connected to light emitters and light receivers. In another example, electroconductive pathways can be wires. In an example, light emitters can receive electrical power through electroconductive channels in an elastomeric polymer. In an example, a smart bra cup can further comprise a plurality of undulating (e.g. sinusoidal, serpentine, or zigzagging) electroconductive pathways which provide electrical power to optical modules. In an example, a smart bra cup can include undulating (e.g. sinusoidal or zigzagging) electroconductive pathways which are in electrical communication with light emitters and light receivers.

In an example, a smart bra cup can comprise a layer of elastic and/or stretchable material, a plurality of optical modules on the layer, and a plurality of undulating electroconductive pathways (e.g. wires or electroconductive polymer pathways) which are connected to the optical modules. In an example, a smart bra cup can comprise a plurality rings of optical modules (e.g. containing light emitters, light receivers, or both) which are connected to a plurality of undulating (e.g. sinusoidal, zigzag, and/or serpentine) electroconductive pathways (e.g. wires or electroconductive polymer). In an example, electroconductive pathways can be made with a flexible and/or elastic polymer (e.g. PDMS) which has been doped, impregnated, and/or coated with electroconductive material (e.g. metal or carbon).

In an example, undulating electroconductive pathways can be made with a flexible and/or elastic polymer (e.g. PDMS) which has been doped, impregnated, and/or coated with electroconductive material (e.g. metal or carbon). In an example, light from light emitters in a first quadrant of a cup can be received by light receivers in a second quadrant of the cup after the light has been transmitted through breast tissue. In another example, optical modules in an upper-outer quadrant of a smart bra cup can be closer together than those in an upper-inner quadrant of the cup.

In an example, a first side of a smart bra cup can be the right side of the cup and a second side of the cup can be the left side of the cup, wherein the two sides are separated by a vertical plane. In another example, a smart bra cup can comprise two expandable chambers and a plurality of optical modules, wherein the expandable chambers have convex lens and/or football shapes when they are expanded, and wherein the longitudinal axes of the convex lens and/or football shapes are substantially parallel to an oblique line from the upper-outer quadrant of the cup to the lower-inner quadrant of the cup.

In an example, light emitters can be on the inner (e.g. right) side of a cup and light receivers can be on the outer (e.g. left) side of the cup, so light emitters and light receivers are separated by a vertical plane, or vice versa. In another example, light from light emitters in a first quadrant of a cup can be received by light receivers in a different vertical-plane in a second quadrant of the cup, after the light has been transmitted through breast tissue. In an example, there can be two gaps in the distribution of optical modules around the perimeter of a smart bra cup, wherein these two gaps are located on a horizontal line (e.g. axis) between the upper quadrants of the cup and the lower quadrants of the cup.

In a traditionally-shaped bra, the upper-outer quadrant of a perimeter of a cup generally has a section with an outward-facing concavity, but in a smart bra this section can have an inward-facing concavity (e.g. opening toward the center of the cup) for better scanning of outer-upper quadrant and the Tail of Spence. In an example, an upper-outer quadrant of the perimeter of a smart bra cup can have an outward-facing concavity, the upper-inner quadrant of the perimeter can have a first section with an outward-facing concavity and a second section with an inward-facing concavity, the lower-inner quadrant of the perimeter can have an inward-facing concavity, and the lower-outer quadrant of the perimeter can have an inward-facing concavity. In an example, an upper-outer quadrant of the perimeter of a smart bra cup can have an inward-facing concavity, the upper-inner quadrant of the perimeter can have a first section with an outward-facing concavity and a second section with an inward-facing concavity, the lower-inner quadrant of the perimeter can have an inward-facing concavity, and the lower-outer quadrant of the perimeter can have an inward-facing concavity.

In an example, a proximal portion of a cup can be less flexible, less elastic, and/or more rigid than a distal portion of a cup so that it resists outward expansion of the expandable chambers and directs their expansion inward toward the breast, thereby causing partial outward flattening of the breast. In an example, a smart bra cup can comprise a plurality of elastic and/or stretchable rings, wherein distal (e.g. farther from the chest wall) rings are more elastic and/or stretchable than proximal (e.g. closer to the chest wall) rings. In another example, a smart bra cup can comprise a proximal portion with a first level of elasticity (or stretchability) and a distal portion with a second level of elasticity (or stretchability), wherein the second level is greater than the first level. In an example, expansion of expandable chambers in a smart bra cup can extend a breast in an anterior direction if the distal portion (e.g. farther to the chest wall) of the bra cup is more elastic than the proximal (e.g. closer to the chest wall) portion of the bra cup.

In another example, a relatively-inelastic band and/or layer can be made with a material which has a lower durometer level or lower Shore value than the material used to make the rest of the cup. In an example, a relatively-inelastic band and/or layer can have lower durometer level or lower Shore value than the rest of the cup. In another example, a smart bra cup can comprise: a first set of optical modules on a first side of a bra cup; a first relatively-inelastic band and/or layer on the first side of the bra cup; a first expandable chamber on the first side of the bra cup; wherein the first set of optical modules are between the first relatively-inelastic band and/or layer and a breast held by the cup; wherein the first relatively-inelastic band and/or layer is between the first set of optical modules and the first expandable chamber; a second set of optical modules on a second side of the bra cup; a second relatively-inelastic band and/or layer on the second side of the bra cup; a second expandable chamber on the second side of the bra cup; wherein the second set of optical modules are between the second relatively-inelastic band and/or layer and the breast; and wherein the second relatively-inelastic band and/or layer is between the second set of optical modules and the second expandable chamber. In an example, a smart bra cup can include a relatively-inelastic band and/or layer which helps to make the thickness of the breast more uniform when compressed, thereby improving the accuracy of optical scanning of the breast for abnormal tissue.

In an example, a cup can comprise a deformable, air-gap-reducing layer between optical modules and the surface of a breast. In an example, a smart bra can further include an pneumatic pressure sensor to help avoid too much pressure on a breast during compression. In an example, a smart bra with a plurality of expandable chambers can include pressure sensors and/or strain sensors to avoid excessive compression of a breast. In an example, a device with optical modules to detect abnormal breast tissue can be embodied in an adhesive patch, sticker, or bandage with optical modules. In an example, a smart bra cup can further comprise disposable adhesive strips (or rings) which are removably attached to the inner layer of the cup and to breast tissue near the chest wall in order to gently engage tissue close to the chest wall. In an example, a smart bra cup can comprise a plurality of optical fibers which transmit light from light emitters to selected locations on the interior of the cup. In an example, a smart bra cup can further comprise optical fibers which emit light from their ends at a plurality of points around the interior concavity of the cup.

In an example, a smart bra cup for detection of abnormal breast tissue (e.g. tumors) can comprise a plurality of optical modules (including light energy emitters and receivers) and thermal energy sensors, wherein thermal interaction between light energy and breast tissue helps in the detection, location, imaging, and/or characterization of abnormal breast tissue. In another example, a smart bra cup for detection of abnormal breast tissue (e.g. tumors) can comprise a plurality of optical modules (including light emitters and receivers) and acoustic modules (including ultrasound emitters and ultrasound detectors).

In another example, a smart bra cup with optical modules can be made from one or more materials selected from the group consisting of: Zinc Oxide Nanoparticles, one-dimensional nanomaterial, zero-dimensional nanomaterial, poly-Ethylene Naphthalate (PEN), poly-Ethylene Terephthalate (PET), a carbon nanomaterial, poly Lactic-co-Glycolic Acid (PLGA), a hydrogel, poly-Glycolic Acid (PGA), poly Methyl Meth-Acrylate (PMMA), poly-Ethlylene Terephthalate (PET), nanocrystals, Transition Metal Dichalcogenide (TMD), organic-inorganic composite/hybrid material, inorganic graphene, silk, graphene, and silicone rubber.

In an example, a smart bra can further comprise other components which are located on the back strap. In another example, a smart bra can be a component of a system which includes a local data processor, a local data transmitter (which are contiguous parts of the smart bra) and a remote (non-contiguous) data processor which receives data from the data transmitter. In an example, a smart bra can further comprise an exterior indicator light (e.g. on the outside of the bra) which lights up when an optical scan of breast tissue is in progress to encourage the wearer to limit movement (and possibly hold their breath for short time) during a scan of a cross-section of a breast.

In an example, a smart bra can further comprise one or more other components selected from the group consisting of: power source (e.g. battery), flexible electroconductive wires and/or textile channels, data processor (local, remote, or both local and remote), wireless data transmitter, wireless data receiver, pressure sensors, motion sensors (e.g. accelerometer and gyroscope), inclinometer, mirrors (e.g. micromirror array), pump and/or impellor (e.g. air or liquid pump or impellor), tubes (e.g. air or liquid conducting tubes), air or liquid reservoir, and electromagnetic actuator.

In an example, a cup or cup insert can be rotated to scan a breast from different orientations. In an example, a device with optical modules to detect abnormal breast tissue can be modular. In an example, a modular cup insert for optical detection of abnormal breast tissue can be inserted into a right-side cup of a smart bra in a first orientation and inserted into a left-side cup of a smart bra in a second (e.g. reflected and/or rotated) orientation. In an example, a wearable device for optical detection of abnormal breast tissue can be a cup insert instead of a cup which is a permanent part of a bra.

In another example, changes in amplitude and/or spectrum of light from the light emitters which are caused by reflection of the light by breast tissue and/or transmission of the light through breast tissue can be analyzed to image breast tissue and/or detect breast cancer. In an example, changes in light from a light emitter which are caused by interaction with breast tissue can be analyzed to image, detect, locate, and/or characterize abnormal breast tissue (e.g. tumors). In another example, changes in the spectrum (e.g. spectral distribution) of light emitted from light emitters and received by light receivers which are caused by transmission of the light through breast tissue can be analyzed to image breast tissue.

In another example, light which has been transmitted through and/or reflected from breast tissue can be analyzed to detect and/or image levels, concentrations, and/or locations of deoxyhemoglobin in breast tissue. In an example, light which has been transmitted through and/or reflected from breast tissue can be analyzed to detect and/or image the locations, sizes, and/or configurations of vasculature sprouting in breast tissue. In an example, a smart bra can be worn for a short period of time on a periodic basis (e.g. annually, monthly, weekly, or daily) in order to obtain a periodic longitudinal time series of optical of breast tissue for identification of changes in tissue composition.

In an example, a smart bra can detect abnormal tissue via Frequency Domain (FD) optical analysis. In an example, a smart bra can use Diffuse Optical Imaging (DOI) to analyze the molecular composition of breast tissue, detect abnormal breast tissue, evaluate the size and shape of abnormal breast tissue, identify selected biometric parameters in breast tissue, identify the location of abnormal breast tissue, and/or image breast tissue. In an example, a smart bra can use Raman scattering to analyze the molecular composition of breast tissue, detect abnormal breast tissue, evaluate the size and shape of abnormal breast tissue, identify selected biometric parameters in breast tissue, identify the location of abnormal breast tissue, and/or image breast tissue.

In an example, light which has been transmitted through and/or reflected from breast tissue and received by light receivers can be analyzed to detect and/or image abnormal breast tissue using one or more methods selected from the group consisting of: Time Reversal Optical Tomography (TROT), changes in the frequency spectrum of light transmitted through a breast, Diffuse Optical Imaging (DOI), Diffuse Optical Tomography (DOT), spectroscopic analysis, analysis of absorption and/or scattering of light transmitted through a breast. Near-Infrared spectroscopy (NIRS), functional Near-Infrared spectroscopy (fNIRS), changes in the intensity or amplitude of light transmitted through a breast, changes in the phase of light transmitted through a breast, Diffuse Correlation spectroscopy (DCS), Carlavian Curve analysis (CCA), machine learning, neural network analysis, broadband spectroscopy, and/or changes in the spectral distribution of light transmitted through a breast.

In an example, a light emitter can be a laser with a narrow pulse width. In an example, a light emitter can be a Light-Emitting Electrochemical Cell (LEC). In an example, a light emitter can be a Monochromatic LED (MLED). In another example, a light emitter can be a Nanoscale LED. In an example, a light emitter can be a Quantum Dot LED (QLED). In another example, a light emitter can be a Resonant Cavity Light Emitting Diode (RCLED). In an example, a light emitter can be a Single Photon Avalanche Diode (SPAD). In an example, a light emitter can be a tunable LED. In an example, a light emitter can be an Active Matrix Organic Light-Emitting Diode (AMOLED). In an example, a light emitter can be an Organic Photovoltaic (OPV). In an example, a light receiver can be an avalanche photo diode (APDs) or PIN photodiode.

In an example, a first set of light emitters can emit light at a first intensity or amplitude level (or at a first time) and a second set of light emitters can emit light at a second intensity or amplitude level (or at a second time). In another example, a light emitter can emit intensity-modulated and/or amplitude-modulated light. In an example, a light emitter can emit light via Alternating Current Electroluminescence (ACEL). In another example, a light emitter in an optical module can transmit light into (and/or onto) breast tissue.

In an example, light emitters can be configured in a checkerboard array. In another example, light emitters can be configured in a star-burst array. In an example, a smart bra cup can further comprise a flexible, elastic, and/or stretchable hexagonal (e.g. honeycomb) grid of light emitters and light receivers. In an example, a smart bra cup can further comprise a helical array of light emitters and light receivers. In an example, a virtual plane which best fits the breast-facing side of an optical module in a cross-section of a cup can be tangential to a radial vector which extends out from the center of the cross-section of the cup.

In an example, an array of optical modules can be arranged in (e.g. distributed along) a hexagonal (e.g. honeycomb) mesh, grid, and/or matrix. In an example, optical modules in an array of optical modules can be activated in a radial sequence. In an example, optical modules on a smart bra cup can be arranged in a radial, hub-and-spoke, or star-burst array comprising six longer rays (or spokes) and six shorter rays (or spokes).

In an example, a smart bra cup can have multiple (e.g. three) rings of optical modules, including an outer ring, a middle ring, and an inner ring. In another example, a smart bra cup can have rings of optical modules, wherein the rings have paisley shapes from a frontal perspective. In an example, an outer ring of optical modules can be around the proximal (e.g. closest to the chest wall) perimeter of a cup. In another example, a smart bra cup can have rings of optical modules, wherein an outer ring is on the proximal perimeter of the cup and inner rings are nested within the outer ring when viewed from a transparent (e.g. two-dimensional) frontal perspective.

In an example, a smart bra cup can have rings of optical modules, wherein the rings are nested from a transparent (e.g. two dimensional) perspective, and wherein outer rings are closer together than inner rings. In another example, a smart bra cup can have three rings of optical modules, wherein an outer ring is on the proximal perimeter of the cup and two inner rings are nested within the outer ring when viewed from a transparent (e.g. two-dimensional) frontal perspective. In an example, light emitters can be configured in nested half rings. In an example, optical modules in an outer ring in an array of (three) nested rings can closer together than optical modules in an inner ring in the array.

In an example, a cross-section of a cup can have a shape whose two-dimensional parametric equation is X=cos(T) and Y=[sin(T)][sinM(T/2)]. In an example, a perimeter of a cross-section of a distal portion of a smart bra cup can have an elliptical, oval, circular, and/or oblate-circular shape. In an example, a perimeter of a smart bra cup can have a convex shape. In an example, a side of an optical module which faces toward the surface of a breast can be arcuate (e.g. concave). In an example, a smart bra cup can have a frustal shape, wherein there is a distal opening on a cup to allow maximum forward expansion of a breast during compression. In an example, a smart bra cup can have a teardrop-shaped perimeter, wherein the point of the teardrop is oriented toward the upper-outer quadrant. In an example, a smart bra cup can have an elliptical, oval, circular, oblate-circular, and/or globular shape.

In an example, individual light emitters and individual light receivers can be distributed around the perimeter of a cup. In another example, light emitters and receivers in a smart bra cup can span between 60% and 85% of the perimeter of the cup. In an example, light emitters and receivers in a smart bra cup can span all of the interior concavity of the cup. In another example, optical modules can be evenly-distributed (e.g. evenly-spaced) around a perimeter of a smart bra cup. In another example, optical modules in a smart bra cup can span between 60% and 85% of the interior concavity of the cup. In an example, optical modules in a smart bra cup can span the entire perimeter of the cup.

In an example, a first light emitter can emit light with a first wavelength and a second light emitter can emit light with a second wavelength. In an example, a first light emitter can emit light with a wavelength in the range of 650 to 750 nm at a first time; a second light emitter can emit light with a wavelength in the range of 750 nm to 850 nm at a second time; and a third light emitter can emit light with a wavelength in the range of 850 nm to 950 nm at a third time. In an example, a first light emitter can emit light with a wavelength in the range of 600 to 900 nm; a second light emitter can emit light with a wavelength in the range of 900 nm to 1200 nm; and a third light emitter can emit light with a wavelength in the range of 1200 nm to 1500 nm.

In an example, a first light emitter can emit light with a wavelength in the range of 600 to 900 nm and a second light emitter can emit light with a wavelength in the range of 900 nm to 1200 nm. In an example, a first light emitter can emit light with a wavelength in the range of 600 to 700 nm; a second light emitter can emit light with a wavelength in the range of 700 nm to 800 nm; and a third light emitter can emit light with a wavelength in the range of 800 nm to 900 nm. In an example, a first light emitter can emit near-infrared light and a second light emitter can emit red light. In an example, a first set of light emitters can emit light at a first frequency and/or wavelength (or in a first spectral range) and a second set of light emitters can emit light at a second frequency and/or wavelength (or in a second spectral range). In an example, a light emitter can emit light at different wavelengths over time, within the range of 600 to 1100 nm.

In an example, a light emitter can emit light at different wavelengths over time selected from the group consisting of: 600, 650, 660, 680, 690, 750, 775, 780, 785, 800, 808, 810, 830, 850, and 1000 nm. In another example, a light emitter can emit light with a wavelength and/or frequency which changes over time. In an example, different light emitters can emit light at different wavelengths. In another example, light emitters can emit light at a frequency and/or wavelength which varies over time. In another example, one or more light emitters in a smart bra cup can be near-infrared light emitters. In an example, one or more light emitters in a smart bra cup can be a green-light lasers. In an example, the wavelengths and/or frequencies of light from light emitters can be changed.

In an example, a first light emitter at a first location in an optical module can emit (a pulse of) light at a first time and a second light emitter at a second location in the optical module can emit (a pulse of) light at a second time. In an example, a first light emitter can emit light at a first time and a second light emitter can emit light at a second time. In an example, optical modules in an array of optical modules can be activated in a linear sequence.

In an example, a light receiver can be a flexible organic photodetector (OPD). In an example, a light receiver can be a photodetector. In an example, a light receiver can be an organic photodiode. In an example, a light receiver can be selected from the group consisting of: photodetector, photoresistor, avalanche photodiode (APD), charge-coupled device (CCD), complementary metal-oxide semiconductor (CMOS), infrared detector, infrared photoconductor, infrared photodiode, light dependent resistor (LDR), optoelectric sensor, photoconductor, photodiode, photomultiplier, and phototransistor. In an example, a light receiver can receive light from a light emitter which is located in a different optical module than the light receiver. In another example, light emitters can be on a first side of a cup and light receivers can be on a second side (e.g. opposite the first side) of the cup. In an example, an optical module can further comprise an opaque light shield between a light emitter and a light receiver.

In another example, a light emitter can emit a beam of light toward a light guide along a vector which is substantially parallel to a side of an optical module and the light guide can redirect (e.g. reflect) this beam of light toward a breast. In another example, a light guide can redirect light from a light emitter. In an example, a movable light guide can pivot and/or rotate around an axle. In an example, a movable light guide in an optical module can have a circular, elliptical, or oval shape. In an example, a movable light guide in an optical module in a smart bra cup can have a square or rectangular shape. In an example, an optical module can comprise a first light emitter, a second light emitter, a movable light guide, and a light receiver.

In an example, an optical module can comprise two light emitters, a light guide, and a light receiver, wherein all of the components are arranged in a line. In an example, an optical module can further comprise a movable light guide. In an example, an optical module can include a light emitter, a movable light guide, and a light receiver. In an example, light from a first light emitter can reflected by a first side of a light guide toward a breast at a first time and light from a second light emitter can be reflected by a second (e.g. opposite) side of the light guide toward the breast at a second time. In an example, movement (e.g. pivoting and/or rotation) of a light guide can change the paths along which light beams from a light emitter travel through breast tissue.

In an example, a light guide can be a digital micromirror device. In an example, a light guide can be a mirror (e.g. micromirror). In an example, a light guide can be a prism. In another example, a movable light guide can be a digital micromirror device. In an example, the angle at which a optical module emits a beam of light into breast tissue can be changed by a movable light guide (e.g. a pivoting and/or rotating mirror) within the optical module.

In another example, a light guide can be a moving (e.g. pivoting or rotating) mirror (e.g. micromirror) which is moved by interaction with a changing electromagnetic field. In an example, a light guide can be pivoted and/or rotated by changes in an electromagnetic field. In another example, an optical module can further comprise a movable reflective light guide which is moved by interaction with a changing electromagnetic field. In an example, an optical module can further comprise two electrodes, wherein changing the power and/or polarity of electrical energy transmitted through the electrodes changes an electromagnetic field which moves (e.g. pivots or rotates) a light guide.

In an example, a movable light guide can reflect and/or refract light from a light emitter at different angles (or along different vectors) toward a breast as the light guide pivots and/or rotates. In an example, a movable light guide in an optical module in a smart bra cup can move (e.g. pivot and/or rotate) in an intermittent manner, thereby causing a beam of light from a light emitter to scan through breast tissue at a sequence of discrete angles (e.g. discrete vectors). In an example, angles between the focal vectors of light emitted from optical modules and the surface of a breast or cup can increase with the distance of the modules from the apex of the concave surface of a breast or cup.

In an example, data concerning transmission of light beams through breast tissue along multiple vectors (e.g. in the same cross-section of the breast) can be compiled to create an image of (e.g. of the cross-section of) the breast. In another example, different optical modules can be activated to transmit pulses of light into a breast along different vectors at different times. In an example, light beams can be transmitted through breast tissue along multiple vectors between pairs of light emitters and light receivers at different locations around the perimeter of a cup.

In another example, light beams can be transmitted through breast tissue along multiple vectors between multiple pairs of optical components, wherein data concerning transmission of these light beams can be compiled and analyzed to image, detect, locate, and/or characterize abnormal tissue in the breast. In an example, multivariate analysis of the intensity and spectral changes of light beams caused by interaction with breast tissue along different vectors can detect abnormal tissue within the breast and where the abnormal tissue is located. In another example, pairs of light emitters and light receivers in different (e.g. opposite) quadrants can be activated at different times to scan breast tissue along different vectors.

In an example, a first set of optical modules can each comprise a light emitter and a second set of optical modules can each comprise a light receiver. In another example, an optical module can have multiple light emitters. In an example, an optical module can have multiple light emitters and a light receiver which are configured in an arcuate array. In another example, an optical module can include a light emitter and a light receiver. In an example, an optical module can include one light emitter and a plurality of light receivers. In another example, an optical module can include one light receiver and a plurality of light emitters. In an example, an optical module can include one light receiver and a plurality of light emitters distributed around the light receiver.

In an example, a smart bra cup can comprise a repeating sequence of light emitters and light receivers distributed around the perimeter of the cup. In an example, a smart bra cup can further comprise a repeating (e.g. e1-e2-r) sequence of light emitters and light receivers around (a portion of) the perimeter of the cup, wherein (e1) is a near-infrared light emitter, (e2) is a visible light emitter, and (r) is a light receiver. In an example, a smart bra cup can further comprise an alternating sequence of light emitters and light receivers around the perimeters of three (vertical plane) cross-sections of the cup.

In an example, a smart bra cup can further comprise an alternating sequence of light emitters and light receivers around nested rings in the cup, wherein the rings are in different two-dimensional planes but appear as nested from a frontal transparent perspective. In an example, one light emitter in a repeating sequence of light emitters can emit red visible light. In an example, optical modules on a smart bra cup can be arranged in a radial, hub-and-spoke, or star-burst array, wherein there is a clockwise sequence of alternating longer rays (or spokes) and shorter rays (or spokes). In an example, there can be an alternating sequence of light emitters and light receivers around each ring in a plurality of nested rings in a smart bra cup.

In an example, a first expandable chamber in a smart bra cup can be expanded by a greater percentage than a second expandable chamber in the smart bra cup. In another example, a plurality of expandable chambers in a smart bra cup can be selectively, independently, and differentially expanded to gently compress a breast held by the cup. In an example, a set of one or more expandable chambers on one side of a smart bra cup can comprise three expandable chambers, wherein a second chamber is between first and third chambers, and wherein the second chamber is larger than the first and third chambers. In another example, a smart bra can comprise a cup with a first set of expandable chambers on a first side of the cup, a second set of expandable chambers on a second side of the cup, an array of light emitters between the first set of expandable chambers and the breast, and an array of light receivers between the second set of expandable chambers and the breast.

In an example, a smart bra cup can comprise: a first set of optical modules on a first side of a bra cup; a first expandable chamber on the first side of the bra cup, wherein the first set of optical modules are configured to be located between the first expandable chamber and a breast held by the cup; a second set of optical modules on a second (e.g. opposite) side of the bra cup; and a second expandable chamber on the second side of the bra cup, wherein the second set of optical modules are configured to be located between the second expandable chamber and the breast. In another example, expandable chambers and optical modules (e.g. including light emitters and light receivers) can be located within the concavity of a cup in a smart bra. In an example, expansion of expandable chambers in a smart bra cup can compress the lateral width of a breast.

In an example, a plurality of expandable chambers in a smart bra cup can be selectively, independently, and differentially expanded by being filled with a compressible substance in order to gently compress a breast held by the cup. In an example, a smart bra cup can comprise: a plurality of expandable chambers on opposite sides (e.g. left and right, upper and lower, or upper-inner and lower-outer) of the cup; a plurality of movable rigid (e.g. low-elasticity) sections (e.g. panels, bands, or strips) on opposite sides (e.g. left and right, upper and lower, or upper-inner and lower-outer) of the cup; and a plurality of optical modules (e.g. further comprising light emitters, light receivers, or both) in optical communication with the breast; wherein the movable rigid sections are between the expandable chambers and the optical modules, wherein the optical modules are between the movable rigid sections and the breast, and wherein filling the expandable chambers with a flowable substance (e.g. a gas, liquid, or gel) pushes the movable rigid sections toward the breast in order to gently compress the breast.

In an example, a smart bra cup can comprise: a plurality of light emitters in the upper-outer and lower-outer quadrants of the cup; a plurality of light receivers in the upper-inner and lower-inner quadrants of the cup; and an expandable chamber in the upper-outer and lower-outer quadrants of the cup, wherein filling this expandable chamber with a flowable substance (e.g. a gas, liquid, or gel) gently compresses a breast held by the cup to decrease the distances between the light emitters and light receivers. In an example, a smart bra cup can further comprise a first expandable chamber in a lower-outer quadrant of the cup and a second expandable chamber in a lower-inner quadrant of the cup, wherein filling these expandable chambers with a flowable substance (e.g. a gas, liquid, or gel) gently lifts, compresses, and flattens a breast held by the cup to improve optical scanning for abnormal breast tissue.

In an example, a smart bra cup can further comprise expandable chambers on opposite sides (e.g. upper and lower sides) of the cup which are expanded by being filled with a flowable substance (e.g. air or a liquid), wherein expansion of these one or more chambers gently compresses the breast for improved optical scanning. In an example, a smart bra cup can further comprise one or more expandable chambers in the lower-outer quadrant of the cup which are expanded by being filled with a flowable substance (e.g. air or a liquid), wherein expansion of these one or more chambers gently compresses the breast for improved optical scanning. In an example, an expandable chamber can be expanded by being filled with a liquid (e.g. saline solution).

In an example, a smart bra cup can comprise two expandable chambers and a plurality of optical modules, wherein the expandable chambers have crescent or banana shapes when they are expanded, and wherein a first expandable chamber is primarily located in the upper-inner quadrant of the cup and a second expandable chamber is primarily located in the lower-outer quadrant of the cup. In an example, an expandable chamber can be shaped like a quarter of a circle, ring, or torus. In an example, an expandable chamber can have a crescent or banana shape. In another example, expandable chambers in a smart bra cup can be shaped like crescents or bananas after they have been expanded. In an example, a smart bra can further comprise one or more other components selected from the group consisting of: power source (e.g. battery), data processor, wireless data transmitter, wireless data receiver, and flowable substance pump.

In another example, a smart bra can comprise: at least one cup which holds a breast; a first piezoelectric contracting band on a first side of the cup; a second piezoelectric contracting band on a second side (e.g. the opposite side) of the cup; a first set of optical modules on the first side of the cup; and a second set of optical modules on the second side of the cup. In an example, a smart bra cup can further comprise a plurality of piezoelectric bands which contract when electrical energy is applied to them. In another example, a smart bra cup can further comprise a plurality of concave piezoelectric bands on the lower half of the cup, wherein these piezoelectric bands contract when electrical energy is applied to them, thereby lifting and slightly flattening the breast held by the cup for improved optical scanning.

In an example, a smart bra cup can further comprise one or more piezoelectric bands in the lower-upper-outer quadrant of the cup which are contracted by application of electromagnetic energy, wherein expansion of these one or more piezoelectric bands gently compresses the breast for improved optical scanning. In an example, a smart bra cup can further comprise one or more piezoelectric bands, sections, and/or layers which are contracted by application of electromagnetic energy in order to apply moderate compression to a breast. In an example, a smart bra cup can further comprise three concave piezoelectric bands on the lower half of the cup at different distances from the chest wall, wherein these piezoelectric bands contract when electrical energy is applied to them, thereby lifting and slightly flattening the breast held by the cup for improved optical scanning.

In an example, a smart bra cup can further comprise a flexible, elastic, and/or stretchable helical (e.g. spiral) grid of electroconductive pathways which connect light emitters and light receivers to a power source. In an example, a smart bra cup can further comprise a plurality of electroconductive pathways which provide electrical power to light emitters and transmit data from light receivers. In an example, a smart bra cup can further comprise electroconductive pathways which provide electrical power to light emitters. In an example, a smart bra cup can have a plurality of elastic electroconductive pathways which are configured in a honeycomb (e.g. hexagonal) grid or mesh.

In an example, a smart bra cup can have radial electroconductive pathways which are connected to light emitters and light receivers. In an example, electroconductive pathways can provide electrical power to light emitters. In an example, light emitters can receive electrical power through channels comprising an elastomeric polymer which has been embedded, impregnated, and/or coated with electroconductive material. In an example, a smart bra cup can further comprise undulating (e.g. sinusoidal or serpentine) wires which connect and power optical modules. In another example, light emitters can receive electrical power through undulating wires which are embedded in an elastomeric polymer.

In an example, a smart bra cup can comprise a plurality of optical modules (e.g. containing light emitters, light receivers, or both) which are connected to a radial (e.g. hub-and-spoke or sunburst) array of undulating (e.g. sinusoidal, zigzag, and/or serpentine) electroconductive pathways (e.g. wires or electroconductive polymer). In another example, an electroconductive pathway can be made with a silicone-based elastomeric polymer (e.g. PDMS) which has been doped, impregnated, or coated with conductive material (e.g. silver or carbon particles). In an example, electroconductive pathways in a smart bra cup can be made with a silicone-based polymer which has been doped, impregnated, and/or coated with electroconductive material.

In another example, a light receiver can receive light from a light emitter which is located in a different quadrant of the cup than the light receiver. In an example, optical modules an upper-outer quadrant of a cup can be closer together than optical modules in other quadrants of the cup. In an example, there can be more optical modules in an upper-outer quadrant of a smart bra cup than in the upper-inner quadrant of the cup.

In an example, a proximal perimeter of a smart bra cup can be substantially elliptical and/or oval, wherein the longest axis of the ellipse and/or oval has an oblique orientation (e.g. from the upper-outer quadrant to the lower-inner quadrant) (e.g. when the person wearing the smart bra is standing up). In an example, light emitters can be on a first side of a cup which includes the upper-inner quadrant of the cup and light receivers can be on a second side of the cup which includes the lower-outer quadrant of the cup, or vice versa, so light emitters and light receivers are separated by an oblique plane between vertical and horizontal planes.

In an example, light emitters can be on the lower side of the cup and light receivers can be on the upper side of the cup, or vice versa, so light emitters and light receivers are separated by a horizontal plane. In an example, light from light emitters on a first side (e.g. left or right) of a cup can be received by light receivers in the same vertical-plane, but on a second side (e.g. the opposite side) of the cup, after the light has been transmitted through breast tissue. In an example, there can be two gaps in the distribution of optical modules around the perimeter of a smart bra cup, wherein these two gaps are located on a vertical line (e.g. axis) between the outer quadrants of the cup and the inner quadrants of the cup.

In another example, all four quadrants of a perimeter of a smart bra cup can have inward-facing concavities. In an example, an upper-outer quadrant of the perimeter of a smart bra cup can have an inward-facing concavity; the upper-inner quadrant of the perimeter can have an outward-facing concavity; the lower-inner quadrant of the perimeter can have an inward-facing concavity; and the lower-outer quadrant of the perimeter can have an inward-facing concavity.

In another example, a central portion of a cup can be less elastic (e.g. higher Young's modulus) and/or more rigid than other portions of the cup. In another example, a smart bra can comprise: a cup which is configured hold a person's breast, wherein the cup includes a proximal portion and a distal portion, wherein the proximal portion is closer to the person's chest wall than the distal portion, and wherein the distal portion is more flexible or elastic than the proximal portion. In an example, a smart bra cup can comprise a proximal (e.g. closer to the chest wall) frustum-shaped portion with a first level of elasticity (or stretchability) and a distal (e.g. farther from the chest wall) dome-shaped portion with a second level of elasticity (or stretchability), wherein the second level is greater than the first level.

In an example, a smart bra cup can comprise: a proximal (e.g. closer to the chest wall) portion of a cup with a first level of elasticity or stretchability; a distal (e.g. farther from the chest wall) portion of the cup with a second level of elasticity or stretchability, wherein the second level is greater than the first level; a first set of optical modules on a first side of a bra cup; a first expandable chamber on the first side of the bra cup, wherein the first set of optical modules are configured to be located between the first expandable chamber and a breast held by the cup; a second set of optical modules on a second side of the bra cup; and a second expandable chamber on a second side of the bra cup, wherein the second set of optical modules are configured to be located between the second expandable chamber and the breast. In an example, there can be variation in the elasticity (e.g. Young's modulus), stretchability, and/or rigidity of different portions of a cup.

In an example, a relatively-inelastic band and/or layer can be relatively-rigid compared to rest of the cup. In an example, a smart bra cup can comprise three layers: an inner layer with optical modules, a middle layer with expandable chambers, and an outer layer which is relatively rigid. In an example, a smart bra cup can comprise: a plurality of expandable chambers; a plurality of movable rigid (e.g. low-elasticity) sections (e.g. panels, bands, or strips); and a plurality of optical modules (e.g. further comprising light emitters, light receivers, or both); wherein the movable rigid sections are between the expandable chambers and the optical modules, and wherein the optical modules are between the movable rigid sections and the breast held by the cup. In an example, a smart bra cup can include a rigid component between optical modules and expandable chambers.

In an example, a smart bra can further comprise pressure sensors. In an example, a smart bra can include pressure sensors which control a pump, wherein the pump is stopped when pressure levels reach a selected level. In an example, expandable chambers can be individually and selectively expanded by different degrees, by different amounts, to different sizes, and/or to different internal pressures, thereby exerting different levels of pressure on different portions of a breast. In another example, a device with optical modules to detect abnormal breast tissue can be embodied in a patch or bandage which is gently adhered to a breast. In an example, there can be a (partially) adhesive ring, band, and/or strip on the perimeter of a smart bra cup, wherein this ring, band, and/or strip is gently adhered to the chest wall. In another example, a smart bra cup can comprise a plurality of optical fibers which transmit light from light emitters around the proximal perimeter of the cup to selected locations on the distal interior of the cup. In another example, a smart bra cup can include undulating (e.g. sinusoidal or zigzagging) optical fibers which guide light to points on the interior of the cup.

In an example, a smart bra cup for detection of abnormal breast tissue (e.g. tumors) can comprise a plurality of optical modules (including light energy emitters and receivers) and thermal energy sensors, wherein the optical modules and the thermal energy sensors are distributed around nested rings on the cup. In an example, a smart bra cup for detection of abnormal breast tissue (e.g. tumors) can comprise a plurality of optical modules (including light energy emitters and receivers) and acoustic modules (including ultrasonic energy emitters and ultrasound detectors), wherein interaction between the light energy and the ultrasonic energy helps in the detection, location, imaging, and/or characterization of abnormal breast tissue.

In an example, optical modules can be attached to, or integrated into, a flexible polymer substrate. In an example, components such as a battery, data processor, and data transmitter can be located on the back strap of a smart bra. In an example, a smart bra can further comprise a local data processer which is in wireless electronic communication with a remote data processor. In an example, a smart bra can further comprise an exterior indicator light (e.g. on the outside of the bra) which lights up when an optical scan of breast tissue is in progress.

In an example, a cup can be a modular and/or removable component of a smart bra which can be removed before the smart bra is washed. In an example, a device with optical modules to detect abnormal breast tissue can be removably-attached to a concave interior of a bra cup by an attachment mechanism (e.g. hook-and-loop material, snap, clip, or magnet) so that it is held in place for optical scanning, but can be removed for washing the smart bra. In an example, a device with optical modules to detect abnormal breast tissue can be inserted into a smart bra cup. In another example, a smart bra cup insert can be removed before the smart bra is washed. In an example, a wearable device with optical modules to detect abnormal breast tissue can be a partial cup insert which is inserted between the cup of a conventional bra and a breast.

In another example, changes in light caused by interaction with breast tissue can be used to identify attributes of abnormal breast tissue such as location, size, shape, and composition. In an example, changes in the intensity (e.g. amplitude) of light emitted from light emitters and received by light receivers which are caused by transmission of the light through breast tissue can be analyzed to evaluate the molecular composition of breast tissue and detect abnormal breast tissue. In another example, changes in the spectrum (e.g. spectral distribution) of light emitted from light emitters and received by light receivers which are caused by transmission of the light through breast tissue can be analyzed to evaluate the molecular composition of breast tissue and detect abnormal breast tissue.

In an example, light which has been transmitted through and/or reflected from breast tissue can be analyzed to detect and/or image levels, concentrations, and/or locations of collagen in breast tissue. In an example, light which has been transmitted through and/or reflected from breast tissue can be analyzed to detect and/or image levels, concentrations, and/or locations of oxygenated hemoglobin in breast tissue.

In an example, a smart bra can be worn periodically in order to obtain a longitudinal time series of optical scans of breast tissue. In an example, a smart bra can detect abnormal tissue via one or more optical methods selected from the group consisting of: Diffuse Optical Imaging (DOI), Diffuse Optical spectroscopic Imaging (DOSI), Diffuse Optical spectroscopy (DOS), Diffuse Optical Tomography (DOT), Frequency-Domain Photon Migration (FDPM), Functional Near-Infrared spectroscopy (fNIRS), Near-Infrared spectroscopy (NIRS), Raman spectroscopy, reflectance Diffuse Optical Tomography (RDOT), Transillumination Imaging (TI), and/or Transmittance Diffuse Optical Tomography (TDOT).

In an example, a smart bra can use Diffuse Optical Tomography (DOT) to analyze the molecular composition of breast tissue, detect abnormal breast tissue, evaluate the size and shape of abnormal breast tissue, identify selected biometric parameters in breast tissue, identify the location of abnormal breast tissue, and/or image breast tissue. In an example, a smart bra can use time of flight Diffuse Optical Tomography (DOT) to analyze the molecular composition of breast tissue, detect abnormal breast tissue, evaluate the size and shape of abnormal breast tissue, identify selected biometric parameters in breast tissue, identify the location of abnormal breast tissue, and/or image breast tissue. In an example, spectroscopic analysis of light which has been transmitted through (and/or reflected from) breast tissue can be done to identify the sizes, configurations, and/or locations of specific biological structures associated with abnormal tissue.

In an example, a smart bra can have a right-side cup and a left-side cup, each having optical modules. In an example, a light emitter can be a coherent light emitter. In an example, a light emitter can be a laser. In another example, a light emitter can be a MicroLED. In an example, a light emitter can be a monochromatic LED. In another example, a light emitter can be a Phosphorescent OLED (PHOLED). In an example, a light emitter can be a red-light laser. In another example, a light emitter can be a Side-Emitting polymer Optical Fiber (SEPOF). In an example, a light emitter can be a Super-Luminescent Light Emitting Diode (SLED). In another example, a light emitter can be a vertical Cavity Surface Emitting Laser (VCSEL). In an example, a light emitter can be an Organic Light Emitting Diodes (OLED). In another example, a light emitter can be an ultraviolet light emitter. In an example, light emitters can be a Light Emitting Diode (LED).

In another example, a light emitter can emit a first pulse of light with a first duration followed by a second pulse of light with a second duration, wherein the second duration is greater than the first duration. In an example, a light emitter can emit light at an angle and/or along a focal vector which varies over time. In an example, a light emitter can emit light within a spectral range which varies over time. In an example, light emitters can emit coherent light.

In an example, light emitters can be configured in a hub-and-spoke array. In an example, a smart bra cup can comprise a radial array (e.g. hub-and-spoke or starburst array) of optical fibers which transmit light from light emitters to selected locations on the interior of the cup. In an example, a smart bra cup can further comprise a flexible, elastic, and/or stretchable helical (e.g. spiral) grid of light emitters and light receivers. In an example, a smart bra cup can further comprise a hexagonal (e.g. honeycomb) grid of light emitters and light receivers. In an example, an array of optical modules can be a radial array, wherein spokes of the array along which optical modules are located extend out radially from an apex of the smart bra cup. In an example, light emitters can be configured in a helical array. In an example, optical modules on a smart bra cup can be arranged in a radial, hub-and-spoke, or star-burst array. In an example, optical modules on a smart bra cup can be arranged in a radial, hub-and-spoke, or star-burst array comprising four longer rays (or spokes) and four shorter rays (or spokes).

In an example, a smart bra cup can have rings of optical modules, wherein the rings have tear-drop shapes from a frontal perspective. In another example, a smart bra cup can have rings of optical modules, wherein the rings have convex cross-sectional shapes from a frontal perspective. In an example, a smart bra cup can comprise nested rings of optical modules. In another example, a smart bra cup can have rings of optical modules, wherein the rings are nested when viewed from a transparent (e.g. two-dimensional) frontal perspective, and wherein an outer ring has more optical modules than an inner ring.

In an example, a smart bra cup can have rings of optical modules, wherein the rings are nested from a transparent (e.g. two dimensional) perspective, and wherein an outer ring and a middle ring are farther apart than the middle ring and an inner ring. In another example, a smart bra cup can have three rings of optical modules, wherein the rings are nested when viewed from a transparent (e.g. two-dimensional) frontal perspective. In an example, light emitters can be configured in nested rings. In an example, optical modules in an outer ring in an array of (three) nested rings can be farther apart than optical modules in an inner ring in the array.

In an example, a perimeter of a cross-section of a cup can have a teardrop cross-sectional shape. In an example, a perimeter of a smart bra cup can have a circular, elliptical, or oval shape. In an example, a perimeter of the portion of a cup which contacts a chest wall can have a teardrop shape, wherein the apex and/or vertex of this perimeter is aligned with the Tail of Spence. In an example, a smart bra cup can have a concave shape. In an example, a smart bra cup can have a generally hemispherical shape. In an example, a smart bra cup can have a teardrop-shaped perimeter. In an example, an apex of a teardrop-shaped smart bra cup can encompass the Tail of Spence.

In an example, light emitters and receivers in a smart bra cup can span the entire perimeter of the cup. In an example, light emitters and receivers in a smart bra cup can span between 50% and 75% of the interior concavity of the cup. In an example, optical modules can be collectively span between 70% and 90% of the perimeter of a smart bra cup. In another example, optical modules in a smart bra cup can span all of the interior concavity of the cup. In an example, optical modules in a smart bra cup can span between 50% and 75% of the perimeter of the cup. In another example, there can be gaps in the distribution of optical modules around the perimeter of a smart bra cup.

In an example, a first light emitter can emit light with a wavelength in the range of 650 to 750 nm; a second light emitter can emit light with a wavelength in the range of 750 nm to 850 nm; and a third light emitter can emit light with a wavelength in the range of 850 nm to 950 nm. In another example, a first light emitter can emit light with a wavelength in the range of 650 to 700 nm; a second light emitter can emit light with a wavelength in the range of 700 nm to 750 nm; and a third light emitter can emit light with a wavelength in the range of 750 nm to 800 nm. In an example, a first light emitter can emit light with a wavelength in the range of 600 to 900 nm at a first time and a second light emitter can emit light with a wavelength in the range of 900 nm to 1200 nm at a second time.

In an example, a first light emitter can emit light with a wavelength in the range of 600 to 800 nm; a second light emitter can emit light with a wavelength in the range of 800 nm to 1000 nm; and a third light emitter can emit light with a wavelength in the range of 1000 nm to 1200 nm. In an example, a first light emitter can emit light with a wavelength in the range of 600 to 700 nm at a first time; a second light emitter can emit light with a wavelength in the range of 700 nm to 800 nm at a second time; and a third light emitter can emit light with a wavelength in the range of 800 nm to 900 nm at a third time. In an example, a first light emitter can emit near-infrared light and a second light emitter can emit green light. In an example, a light emitter can emit light at a constant frequency and/or in a constant spectral range.

In an example, a light emitter can emit light at different wavelengths over time, within the range of 600 to 1100 nm. In an example, a light emitter can emit light at different wavelengths at different times. In an example, a light emitter can emit light with a wavelength and/or frequency which changes in a repeated cyclical pattern over time. In an example, different light emitters can emit light with different wavelengths, within the range of 600 to 1100 nm. In an example, light emitters can emit near-infrared (NIR) light. In another example, one or more light emitters in a smart bra cup can be multi-wavelength lasers. In an example, the wavelengths and/or frequencies of light from light emitters can be varied in a repeated pattern. In another example, the wavelengths and/or frequencies of light from light emitters can be changed in periodic manner.

In an example, a first light emitter at a first location on a bra cup can emit (a pulse of) light at a first time and a second light emitter at a second location on the bra cup can emit (a pulse of) light at a second time. In another example, different optical modules can be activated at different times to isolate different optical pathways. In an example, optical modules in an array of optical modules can be activated in a circumferential sequence.

In an example, a light receiver can be a photoconductor. In an example, a light receiver can be a photoreceptor. In an example, a light receiver can be an organic phototransistor. In an example, a light receiver can have an organic photoactive channel layer, a dielectric layer, and electrodes. In an example, a light receiver can receive light from a light emitter after this light has been transmitted through (and/or been reflected by) breast tissue. In an example, light from light emitters on a first side (e.g. left or right) of a cup can be received by light receivers on a second side (e.g. the opposite side) of the cup after the light has been transmitted through breast tissue. In an example, an optical module can further comprise an opaque light shield between a light emitter and a light receiver, wherein this light shield blocks light from going directly from the emitter to the receiver without being transmitted through breast tissue.

In an example, a light guide can be a lens. In an example, a light guide can reflect light from a light emitter. In another example, a movable light guide can pivot and/or rotate. In an example, a movable light guide in an optical module can pivot and/or rotate around a central (longitudinal) axis. In another example, a movable light guide in an optical module in a smart bra cup can have a circular, elliptical, or oval shape. In an example, an optical module can comprise a light receiver, a movable light guide, a first light emitter to one side of the light guide, and a second light emitter to the other side (e.g. opposite side) of the light guide, wherein light from the first light emitter is reflected by a first side of the light guide toward a breast, and wherein light from the second light emitter is reflected by a second (e.g. opposite) side of the light guide toward the breast.

In another example, an optical module can comprise: a first light emitter; a second light emitter; a light receiver; and a movable light guide (e.g. movable light reflector or refractor). In an example, an optical module can further comprise a movable reflective light guide. In an example, an optical module can include both a light emitter and a movable light guide. In an example, light from a light emitter can be redirected (e.g. reflected and/or refracted) by a light guide before it is transmitted into (and/or onto) a breast. In an example, movement (e.g. pivoting and/or rotation) of a light guide can cause beams of light from a light emitter to scan different sections and/or locations of a breast at different times.

In an example, a light guide can be a digital micromirror device. In an example, a light guide can be a moving (e.g. pivoting or rotating) prism. In an example, a movable light guide can be a digital micromirror device. In an example, a movable light guide can be a movable micromirror. In an example, a light guide (e.g. micromirror) in an optical module can pivot and/or rotate around an axis (e.g. an axle) as it is exposed to an electromagnetic field. In an example, a light guide can be a moving (e.g. pivoting or rotating) micromirror array which is moved by interaction with a changing electromagnetic field. In an example, a light guide can be pivoted and/or rotated by changes in an electromagnetic field created by electrodes in the optical module. In an example, an optical module can further comprise electrodes, wherein transmission of electrical energy through the electrodes creates an electromagnetic field which moves (e.g. pivots or rotates) a movable light guide.

Although light energy is significantly scattered and/or diffused through the depth of breast tissue, multivariate analysis of light transmitted through multiple intersecting vectors between multiple pairs of light emitters and light receivers can provide parallel data which increases optical scanning the accuracy. In another example, a movable light guide can reflect and/or refract light from a first light emitter and/or a second light emitter at different angles (or along different vectors) toward a breast as the light guide pivots and/or rotates. In an example, a smart bra can include a plurality of electromagnetic actuators which change the angles and/or focal vectors of light beams which scan through breast tissue.

In another example, collecting data on changes in transmitted light from a large number of different light pathways (e.g. scan vectors) enables more-accurate detection of abnormal tissue and identification of tissue attributes (such as location, size, shape, and composition). In an example, data concerning transmission of light beams through breast tissue along multiple vectors (e.g. in the same cross-section of the breast) can be compiled and analyzed to image, detect, locate, and/or characterize abnormal tissue in the breast. In another example, light beams can be transmitted through breast tissue along multiple vectors between pairs of light emitters and light receivers at different locations around the perimeter of a cup, wherein data concerning transmission of these light beams can be compiled to create a two-dimensional cross-sectional images and/or a three-dimensional image of the breast.

In an example, light beams can be transmitted through breast tissue along multiple vectors between multiple pairs of optical components. In an example, light beams can be transmitted through breast tissue along multiple vectors between multiple optical components. In an example, optical modules can further comprise a plurality of movable mirrors which change the vectors of light rays from a plurality of light emitters. In an example, the angles at which an array of optical modules (e.g. including light emitters) direct light into breast tissue can be changed (e.g. controlled) by an array of movable micromirrors.

In an example, a first set of optical modules can include light emitters and a second set of light emitters can include light receivers. In an example, an optical module can have multiple light emitters which emit light at different times. In an example, an optical module can have multiple light emitters and a light receiver which are configured in a straight line. In an example, an optical module can include a light emitter and a light receiver. In another example, an optical module can include one light emitter and a plurality of light receivers which are evenly-distributed around the light emitter. In an example, an optical module can include one light receiver and at least four light emitters which are evenly-distributed around the light receiver. In another example, an optical module can include only a light emitter.

In an example, a smart bra cup can further comprise a repeating (e.g. e1-e2-r) sequence of light emitters and light receivers around (a portion of) the perimeter of the cup, wherein (e1) is a light emitter with first wavelength, (e2) is a light emitter with second wavelength, and (r) is a light receiver. In another example, a smart bra cup can further comprise a repeating (e.g. e1-e2-r) sequence of light emitters and light receivers around (a portion of) the perimeter of the cup, wherein (e1) is a near-infrared light emitter, (e2) is a green light emitter, and (r) is a light receiver. In an example, a smart bra cup can further comprise an alternating sequence of light emitters and light receivers around the perimeters of four (vertical plane) cross-sections of the cup. In an example, a smart bra cup can further comprise an alternating sequence of light emitters and light receivers around a cross-sectional perimeter of the cup. In an example, one light emitter in a repeating sequence of light emitters can emit near-infrared light. In an example, there can be a repeating sequence of two light emitters and a light detector around a perimeter of a smart bra cup, wherein the two light emitters emit light at different wavelengths.

In an example, a breast can be compressed into a flatter configuration (for better analysis by transmitted light) by greater expansion of a first expandable chamber in a smart bra cup relative to a second expandable chamber in a cup, wherein the first expandable chamber is closer to the center of the cup. In an example, a first side of an expandable chamber can be more elastic than a second side of the expandable chamber, wherein the first side faces toward a person's breast. In an example, a plurality of expandable chambers on either side (e.g. upper and lower) of a cup can be selectively, independently, and differentially expanded to gently compress a breast held by the cup. In an example, a side and/or surface of an expandable chamber which faces toward the breast can have a lower Young's modulus than the side and/or surface of the component which faces away from the breast.

In an example, a smart bra can comprise: at least one cup which holds a breast; a first expandable chamber on a first side of the cup; a second expandable chamber on a second side (e.g. the opposite side) of the cup; a first set of optical modules on the first side of the cup; and a second set of optical modules on the second side of the cup. In another example, an expandable chamber can be expanded by a hydraulic mechanism. In an example, expandable chambers which are closer to the center of a cup can be expanded to a greater extent than components which are farther from the center of the cup. In another example, gentle and partial outward flattening of the breast by a smart bra can be less than flattening caused by traditional mammography, but still be sufficient to enable accurate optical scanning of the breast.

In another example, a plurality of expandable chambers in a smart bra cup can be selectively, independently, and differentially inflated to gently compress a breast held by the cup. In an example, a smart bra cup can comprise: a plurality of light emitters in the upper-outer quadrant of the cup; a plurality of light receivers in the lower-inner quadrant of the cup; a first expandable chamber in the upper-outer quadrant of the cup; and a second expandable chamber in the lower-inner quadrant of the cup, wherein filling these expandable chambers with a flowable substance (e.g. a gas, liquid, or gel) gently compresses a breast held by the cup to decrease the distances between the light emitters and light receivers.

In an example, a smart bra cup can comprise: a plurality of light emitters in the lower-outer and lower-inner quadrants of the cup; a plurality of light receivers in the upper-outer and upper-inner quadrants of the cup; and an expandable chamber in the lower-outer and lower-inner quadrants of the cup, wherein filling this expandable chamber with a flowable substance (e.g. a gas, liquid, or gel) gently lifts, compresses, and flattens a breast held by the cup to decrease the distances between the light emitters and light receivers. In an example, a smart bra cup can further comprise an expandable chamber in the lower-outer and lower-inner quadrants of the cup, wherein filling this expandable chamber with a flowable substance (e.g. a gas, liquid, or gel) gently lifts, compresses, and flattens a breast held by the cup to improve optical scanning for abnormal breast tissue.

In an example, a smart bra cup can further comprise expandable chambers on opposite sides (e.g. right and left sides) of the cup which are expanded by being filled with a flowable substance (e.g. air or a liquid), wherein expansion of these one or more chambers gently compresses the breast for improved optical scanning. In an example, a smart bra cup can further comprise one or more expandable chambers in the lower-inner quadrant of the cup which are expanded by being filled with a flowable substance (e.g. air or a liquid), wherein expansion of these one or more chambers gently compresses the breast for improved optical scanning. In an example, expandable chambers can be expanded by being filled with a flowable substance (e.g. a gas, liquid, or gel).

In an example, a smart bra cup can comprise two expandable chambers and a plurality of optical modules, wherein the expandable chambers have convex lens and/or football shapes when they are expanded, and wherein a first expandable chamber is primarily located in the upper-inner quadrant of the cup and a second expandable chamber is primarily located in the lower-outer quadrant of the cup. In an example, an expandable chamber can be shaped like a section of a circle, ring, or torus. In an example, an expandable chamber can have a crescent, partial moon, and/or banana shape. In an example, expandable chambers in a smart bra cup can be shaped like convex lenses and/or footballs after they have been expanded. In an example, a smart bra can further comprise other components selected from the group consisting of: power source, data processor, wireless data transmitter, wireless data receiver, air pump, and liquid pump.

In an example, a smart bra cup can comprise: nested rings of optical modules; undulating electroconductive pathways which connect and provide power to the optical modules; and piezoelectric contracting rings and/or bands. In another example, a smart bra cup can further comprise a plurality of piezoelectric bands which contract when electrical energy is applied to them, wherein a first subset of the piezoelectric bands is in the upper half of the cup and a second subset of the piezoelectric bands is on the lower half of the cup. In an example, a smart bra cup can further comprise a plurality of concave piezoelectric bands on the lower half of the cup at different distances from the chest wall, wherein these piezoelectric bands contract when electrical energy is applied to them, thereby lifting and slightly flattening the breast held by the cup for improved optical scanning.

In another example, a smart bra cup can further comprise one or more piezoelectric bands in the lower-outer quadrant of the cup which are contracted by application of electromagnetic energy, wherein expansion of these one or more piezoelectric bands gently compresses the breast for improved optical scanning. In another example, a smart bra cup can further comprise one or more piezoelectric bands on opposite sides (e.g. upper and lower sides) of the cup which are contracted by application of electromagnetic energy, wherein expansion of these one or more piezoelectric bands gently compresses the breast for improved optical scanning. In an example, a smart bra cup can include piezoelectric contracting bands and/or layers for moderate compression of a breast to improve the accuracy of optical scanning.

In an example, a smart bra cup can further comprise a helical (e.g. spiral) grid of electroconductive pathways which connect light emitters and light receivers to a power source. In an example, a smart bra cup can further comprise a plurality of helical electroconductive pathways which provide electrical power to optical modules. In an example, a smart bra cup can have a plurality of elastic electroconductive pathways which are configured in nested (e.g. concentric) rings. In an example, a smart bra cup can have a plurality of elastic electroconductive pathways which are configured in a helical and/or spiral pattern. In an example, a smart bra cup can have rings of electroconductive pathways which are connected to light emitters and light receivers. In an example, electroconductive pathways can transmit data from light receivers to a data processor.

In an example, a smart bra cup can comprise: nested rings of light emitters and light receivers; and undulating electroconductive pathways connected to the light emitters and light receivers. In an example, a smart bra cup can further comprise undulating electroconductive pathways which connect and power optical modules. In an example, light emitters can receive electrical power through undulating (e.g. undulating, wavy, zigzag, and/or sinusoidal) wires (e.g. microwires or nanowires).

In another example, a smart bra cup can comprise a plurality of optical modules (e.g. containing light emitters, light receivers, or both) which are connected to a plurality of undulating (e.g. sinusoidal, zigzag, and/or serpentine) electroconductive pathways (e.g. wires or electroconductive polymer). In an example, an electroconductive pathway can be made with an elastomeric polymer which has been doped, impregnated, or coated with conductive material. In another example, light emitters can receive electrical power through carbon nanotubes in an elastomeric polymer (e.g. PDMS).

In another example, light emitters can be closer together in the upper-outer quadrant of the cup. In an example, optical modules in an upper-outer quadrant of a smart bra cup can be closer together than those in an lower-inner quadrant of the cup. In an example, there can be more optical modules in an upper-outer quadrant of a smart bra cup than in the lower-inner quadrant of the cup. In an example, a smart bra cup can comprise two expandable chambers and a plurality of optical modules, wherein the expandable chambers have crescent or banana shapes when they are expanded, and wherein the longitudinal axes of the crescent or banana shapes are substantially parallel to an oblique line from the upper-outer quadrant of the cup to the lower-inner quadrant of the cup.

In an example, light emitters can be on a first side of a cup which includes the lower-outer quadrant of the cup and light receivers can be on a second side of the cup which includes the upper-inner quadrant of the cup, or vice versa, so light emitters and light receivers are separated by an oblique plane between vertical and horizontal planes. In an example, light from light emitters in a first quadrant of a cup can be received by light receivers in the same vertical-plane, but in a different quadrant of the cup, after the light has been transmitted through breast tissue. In an example, light from light emitters on a first side (e.g. left or right) of a cup can be received by light receivers in a different vertical-plane on a second side (e.g. the opposite side) of the cup, after the light has been transmitted through breast tissue. In an example, there can be two gaps in the distribution of optical modules around the perimeter of a smart bra cup, wherein these two gaps are located on an oblique line (e.g. axis) between the upper-outer quadrant of the cup and the lower-inner quadrant of the cup.

In an example, an upper-outer quadrant of a perimeter of a smart bra cup can have a first section with an inward-facing concavity and a second section with an outward-facing concavity, but the other three quadrants of the perimeter of the cup have only inward-facing concavities. In an example, an upper-outer quadrant of the perimeter of a smart bra cup can have an inward-facing concavity; the upper-inner quadrant of the perimeter can have an inward-facing concavity; the lower-inner quadrant of the perimeter can have an inward-facing concavity; and the lower-outer quadrant of the perimeter can have an inward-facing concavity.

In an example, a distal portion of a smart bra cup (e.g. farther from the chest wall) can be more elastic and/or stretchable than a proximal portion of the cup (e.g. closer to the chest wall) to facilitate gentile compression and expansion of the breast for better optical scanning. In an example, a smart bra can further comprise reinforcing wire (or rigid polymer bands) in a smart bra cup can help to flatten the breast to improve optical scanning. In an example, a smart bra cup can comprise a proximal (e.g. closer to the chest wall) portion with a first level of elasticity (or stretchability) and a distal (e.g. farther from the chest wall) portion with a second level of elasticity (or stretchability), wherein the second level is greater than the first level, and a plurality of expandable chambers between the proximal portion and the breast. In another example, an outer layer of a smart bra cup can restrict outward expansion of expandable chambers so that expansion of these components is directly primarily inward toward the breast.

In an example, a proximal portion of a smart bra cup can be reinforced with wires, strips, or bands to make it less flexible and more rigid. In another example, a relatively-inelastic band and/or layer can be relatively-inelastic compared to the rest of cup. In an example, a smart bra cup can comprise three layers: an inner layer with optical modules, a middle layer with expandable chambers, and an outer layer which is relatively rigid, wherein the rigidity of the outer layer directs expansion of the expandable chambers inward to gently compress the breast for better optical scanning. In another example, a smart bra cup can further comprise relatively inelastic components (e.g. wires or plastic strips) which are embedded in some portions of the cup. In an example, a cup can comprise a deformable gel layer between optical modules and the surface of a breast.

In an example, a smart bra can further include an optical pressure sensor to help avoid too much pressure on a breast during compression. In an example, a smart bra cup can include one or more pressure sensors which measure the pressure and/or force applied to a breast by expansion of expandable chambers. In an example, expansion of expandable chambers can be controlled (e.g. limited) based on pressure levels on those chambers. In an example, a smart bra can further comprise (partially) adhesive strips (e.g. around a perimeter of a cup) to help keep a cup in the same position and enable scanning closer to the chest wall. In an example, a smart bra cup can comprise a plurality of optical fibers (e.g. optical pathways, tubes, channels, or threads) which transmit light from light emitters to selected locations on the interior of the cup. In an example, a smart bra cup can further comprise flexible optical fibers which guide light from light emitters to selected light-exiting points on the interior of the cup.

In an example, a smart bra cup for detection of abnormal breast tissue (e.g. tumors) can comprise a plurality of optical modules (including light energy emitters and receivers) and thermal energy sensors. In an example, a smart bra cup for detection of abnormal breast tissue (e.g. tumors) can comprise a plurality of optical modules (including light emitters and receivers) and acoustic modules (including ultrasound emitters and ultrasound detectors), wherein the optical modules and the acoustic modules are distributed around nested rings on the cup.

In an example, a smart bra cup with optical modules can be made from one or more materials selected from the group consisting of: poly-Imide (PI), parylene, a perovskite, poly-Lactic Acid (PLA), acrylic, polymer and non-fullerene acceptor composite/hybrid, black phosphorus, poly-Styrene (PS), poly-Ethylene Glycol Di-Acrylate (PEGDA), cellulose, poly-Urethane (PU), chitosan, quantum dots, colloidal quantum, shape Memory Photonic Crystal Fiber (SMPF), and organic material. In another example, optical modules can be printed on an elastomeric substrate.

In an example, a smart bra can further comprise a local data processor which is located on the back strap. In another example, a smart bra can include a data transmitter which transmits data from the light receivers to a remote data processor, wherein this data is analyzed in the remote data processor to detect abnormal breast tissue. In an example, a smart bra can include an exterior indicator light (e.g. on the outside of the bra) which lights up when an optical scan of breast tissue is in progress to encourage the wearer to limit movement (and possibly hold their breath for short time) during a scan.

In another example, a cup insert can be rotated and/or flipped between being used on a right breast and being used on a left breast. In another example, a device with optical modules to detect abnormal breast tissue can be removably-attached to the concave interior of a smart bra cup (e.g. by hook-and-loop material) so that it is held in place for optical scanning, but can be removed for washing the smart bra without exposing optical modules (or other electronics) to water and soap. In an example, a device with optical modules to detect abnormal breast tissue can be removably inserted into a pocket, pouch, or other opening in a bra cup. In an example, a wearable device for optical detection of abnormal breast tissue can be inserted into the cup of a conventional bra, thereby giving a conventional bra the functionality of a smart bra. In an example, optical components of a smart bra cup can be removed before the bra is washed.

In an example, changes in light caused by interaction with breast tissue can be analyzed to image, detect, locate, and/or characterize abnormal breast tissue (e.g. tumors). In an example, changes in the intensity and/or spectral distribution of light caused by transmission through breast tissue can be analyzed to create a (3D) image which shows levels and/or concentrations of biological substances (e.g. markers) which are associated with abnormal tissue. In an example, light which has been transmitted through and/or reflected from breast tissue can be analyzed to detect and/or image the locations, sizes, and/or configurations of extracellular matrix structures in breast tissue. In another example, light which has been transmitted through and/or reflected from breast tissue can be analyzed to detect and/or image levels, concentrations, and/or locations of oxygen in breast tissue. In an example, light which has been transmitted through and/or reflected from breast tissue can be analyzed to detect and/or image levels, concentrations, and/or locations of hemoglobin in breast tissue.

In another example, a smart bra can detect abnormal tissue via Continuous Wave (CW) optical analysis. In an example, a smart bra can detect abnormal tissue via Time Domain (TD) optical analysis. In an example, a smart bra can use Near-Infrared Spectroscopy (NIRS) to analyze the molecular composition of breast tissue, detect abnormal breast tissue, evaluate the size and shape of abnormal breast tissue, identify selected biometric parameters in breast tissue, identify the location of abnormal breast tissue, and/or image breast tissue. In an example, light from a light emitter can be received by multiple light receivers and data from these multiple light receivers can be jointly analyzed to image, detect, locate, and/or characterize abnormal breast tissue.

In an example, a smart bra for optical detection of abnormal breast tissue can comprise: a bra with cups which are configured to hold a person's breasts; wherein at least one of the cups has a plurality of optical modules; wherein the plurality of optical modules further comprises light emitters and light receivers; wherein light from the light emitters is received by the light receivers after the light has been transmitted through and/or reflected by breast tissue; and wherein light received by the light receivers is analyzed to detect, locate, image, and/or characterize abnormal breast tissue.

In an example, a plurality of optical modules can be arranged in rings. In an example, the rings can be nested from a frontal perspective. In an example, a plurality of optical modules can be arranged in a radial array. In an example, a radial array can be a starburst array. In an example, a radial array can be a hub-and-spoke array. In an example, a plurality of optical modules can be arranged in a honeycomb grid.

In an example, optical modules in a plurality of optical modules can each have both a light emitter and a light receiver. In an example, optical modules in a plurality of optical modules can each have two light emitters and a light receiver. In an example, two light emitters can emit light with different wavelengths. In an example, two light emitters can emit light at different times. In an example, a first set of optical modules in a plurality of optical modules can each have a light emitter but no light receiver and a second set of optical modules in the plurality of optical modules can each have a light receiver but no light emitter.

In an example, each optical module in a plurality of optical modules can have a light emitter and a movable light guide. In an example, a movable light guide can reflect and/or refract a beam of light from a light emitter toward a breast. In an example, a light guide can be a movable mirror. In an example, each optical module in a plurality of optical modules can have a light emitter, a movable light guide, and a light receiver. In an example, each optical module in a plurality of optical modules can have a first light emitter, a second light emitter, a movable light guide, and a light receiver. In an example, a movable light guide can be located between a first light emitter and a second light emitter.

In an example, a smart bra can further comprise a plurality of expandable chambers, wherein expansion of the expandable chambers compresses a breast. In an example, a smart bra can further comprise a plurality of piezoelectric contracting bands or layers, wherein contraction of the piezoelectric contracting bands or layers compresses a breast.

The lower portion of FIG. 1 shows an opaque frontal view of a smart bra 101 for optical detection of abnormal breast tissue, wherein the smart bra has two cups (including cup 102) which are configured to hold a person's breasts. The upper portion of FIG. 1 shows a close-up frontal view of a cross-section of this cup, illustrating optical modules (including optical modules 105 and 106) around a perimeter 104 of the cup. It is to be understood that there can be additional optical modules (e.g. additional rings of optical modules) in other cross-sections of this cup even though they are not shown in this single cross-sectional view.

In a traditionally-shaped bra, the upper-outer quadrant of a perimeter of a cup generally has a section with an outward-facing concavity (shown here for comparison only as dotted line 103). In contrast, in this smart bra, this section has an inward-facing concavity. An outward-facing concavity in this context is defined as a concavity which opens away from the center of the cup and an inward-facing concavity is defined as a concavity which opens toward the center of the cup. Having an inward-facing concavity for this section enables the smart bra to scan more of the upper-outer quadrant (including the Tail of Spence) of the breast than would be possible with a traditionally-shaped bra. The upper-outer quadrant of a breast is the quadrant which is most-likely to develop abnormal tissue (e.g. tumors).

In an example, an optical module can include a light emitter and a light receiver. The light emitter transmits light into (and/or onto) breast tissue. The light receiver receives this light after the light has been transmitted through (and/or been reflected by) breast tissue. Changes in the light caused by interaction with the breast tissue are analyzed to image, detect, locate, and/or characterize abnormal breast tissue (e.g. tumors). In an example, the light receiver can receive light from a light emitter which is located in the same optical module as the light receiver. In an example, a light receiver can receive light from a light emitter which is located in a different optical module than the light receiver. In another example, individual light emitters and individual light receivers can be distributed around the perimeter of a cup in various configurations instead of multi-component optical modules. Relevant variations discussed elsewhere in this disclosure or in priority-linked disclosures can also be applied to this example.

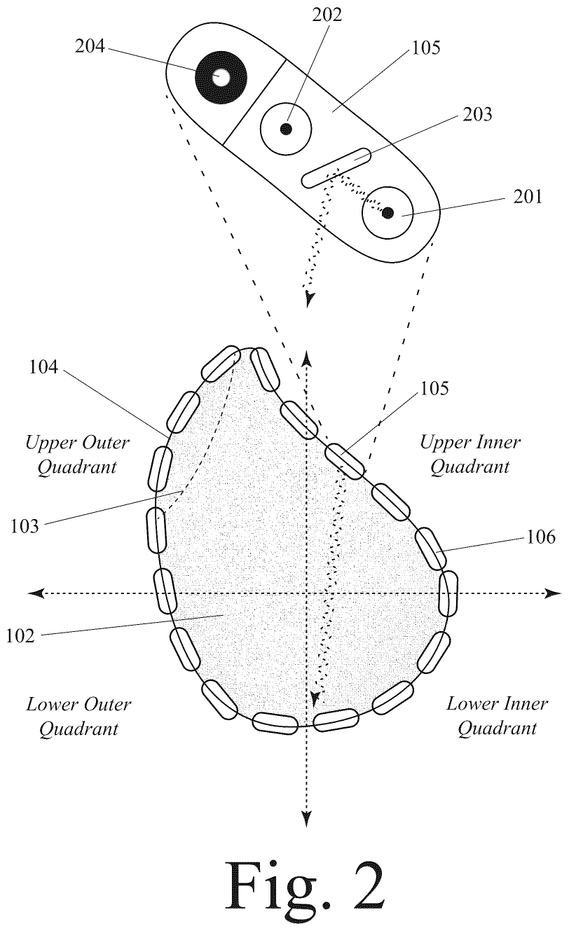

The lower portion of FIG. 2 shows a frontal view of a cross-section of the smart bra cup which was introduced in FIG. 1 at a first time. At this time, optical module 105 is transmitting a beam of light through breast tissue along a first vector. The upper portion of FIG. 2 shows a close-up cross-sectional view of optical module 105. Optical module 105 further comprises: a first light emitter 201; a second light emitter 202; a light receiver 204; and a movable light guide (e.g. movable light reflector or refractor) 203. In this example, light from the first light emitter is being reflected by the light guide toward the breast. In an example, the light guide can be a movable micromirror. In this example, light from the first light emitter is received by a light receiver in a different optical module after it has been transmitted through breast tissue. In this example, light from the first light emitter is received by a light receiver in an optical module in a different quadrant, after this light has been transmitted through breast tissue. Relevant variations discussed elsewhere in this disclosure or in priority-linked disclosures can also be applied to this example.

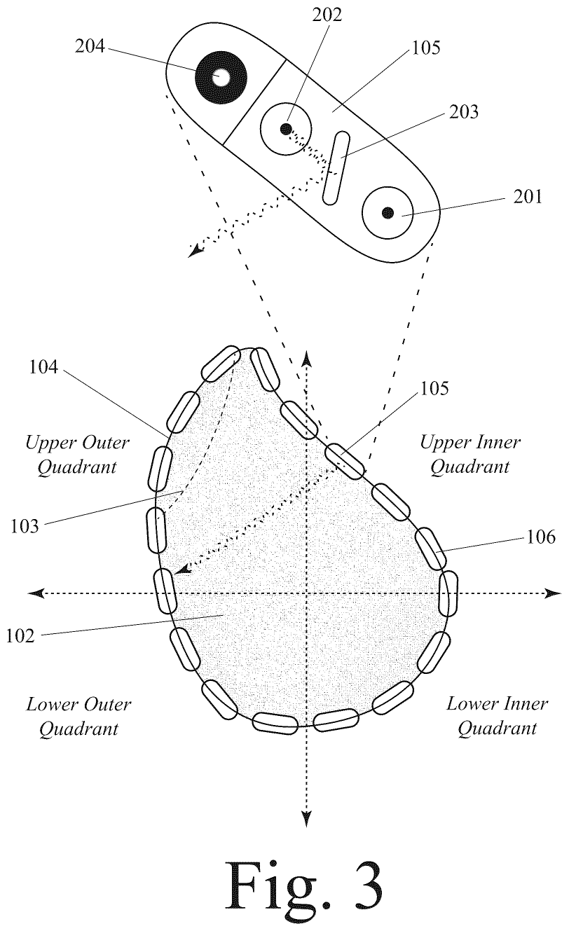

The lower portion of FIG. 3 shows a frontal view of a cross-section of the smart bra cup which was introduced in FIG. 1 at a second time. At this time, optical module 105 is transmitting a beam of light through breast tissue along a second vector. The upper portion of FIG. 3 shows a close-up cross-sectional view of optical module 105. Optical module 105 further comprises: a first light emitter 201; a second light emitter 202; a light receiver 204; and a movable light guide (e.g. movable light reflector or refractor) 203. In this example, the light guide has been moved and light from the second light emitter is now being reflected by the light guide toward the breast. In an example, the light guide can be a movable micromirror. In this example, light from the second light emitter is received by a light receiver in a different optical module after it has been transmitted through breast tissue. In this example, light from the second light emitter is received by a light receiver in an optical module in a different quadrant, after this light has been transmitted through breast tissue. Relevant variations discussed elsewhere in this disclosure or in priority-linked disclosures can also be applied to this example.

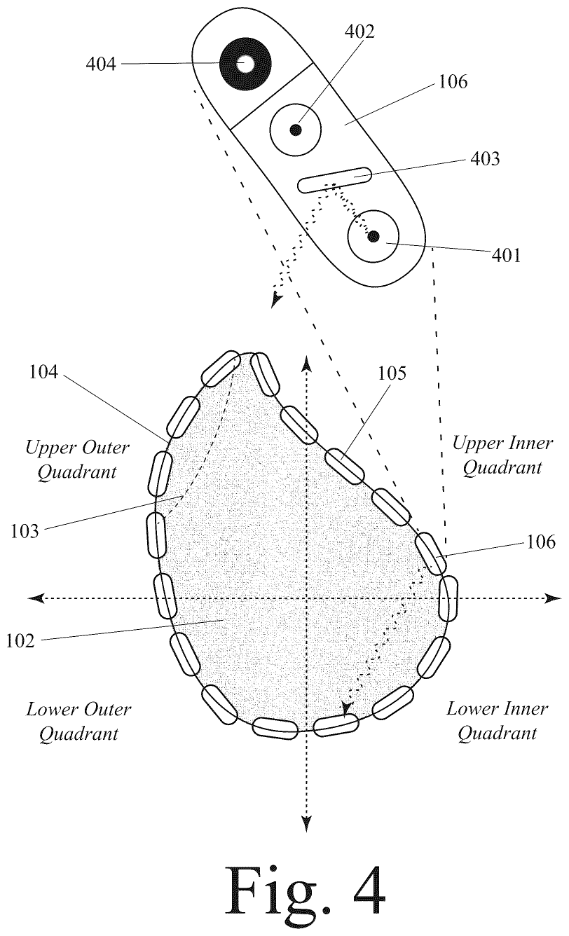

The lower portion of FIG. 4 shows a frontal view of a cross-section of the smart bra cup which was introduced in FIG. 1 at a third time. At this time, a different optical module (optical module 106) is now transmitting a beam of light through breast tissue along a third vector. The upper portion of FIG. 4 shows a close-up cross-sectional view of optical module 106. Optical module 106 further comprises: a third light emitter 401; a fourth light emitter 402; a light receiver 404; and a movable light guide (e.g. movable light reflector or refractor) 403. In this example, light from the third light emitter is being reflected by the light guide toward the breast. In an example, the light guide can be a movable micromirror. In this example, light from the third light emitter is received by a light receiver in a different optical module after it has been transmitted through breast tissue. In this example, light from the third light emitter is received by a light receiver in an optical module in a different quadrant, after this light has been transmitted through breast tissue. Relevant variations discussed elsewhere in this disclosure or in priority-linked disclosures can also be applied to this example.

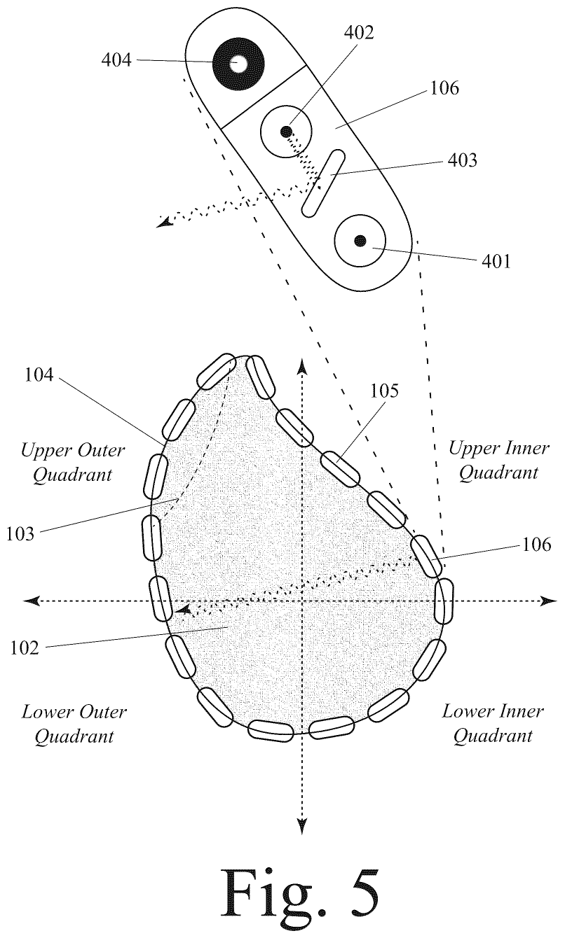

The lower portion of FIG. 5 shows a frontal view of a cross-section of the smart bra cup which was introduced in FIG. 1 at a fourth time. At this time, optical module 106 is transmitting a beam of light through breast tissue along a fourth vector. The upper portion of FIG. 3 shows a close-up cross-sectional view of optical module 106. Optical module 106 further comprises: a third light emitter 401; a fourth light emitter 402; a light receiver 404; and a movable light guide (e.g. movable light reflector or refractor) 403. In this example, the light guide has been moved and light from the fourth light emitter is now being reflected by the light guide toward the breast. In an example, the light guide can be a movable micromirror. In this example, light from the fourth light emitter is received by a light receiver in a different optical module after it has been transmitted through breast tissue. In this example, light from the fourth light emitter is received by a light receiver in an optical module in a different quadrant, after this light has been transmitted through breast tissue.

In an example, data concerning transmission of light beams through breast tissue along multiple vectors (e.g. in the same cross-section of the breast), as shown in FIGS. 2 through 5, can be compiled to create an image of (e.g. of the cross-section of) the breast. In an example, data concerning transmission of light beams through breast tissue along multiple vectors (e.g. in the same cross-section of the breast), as shown in FIGS. 2 through 5, can be compiled and analyzed to image, detect, locate, and/or characterize abnormal tissue in the breast. In an example, data concerning transmission of light beams through breast tissue along multiple vectors in multiple cross-sections of the breast can be compiled to create a three-dimensional model (e.g. three-dimensional image) of the breast. Relevant variations discussed elsewhere in this disclosure or in priority-linked disclosures can also be applied to this example.

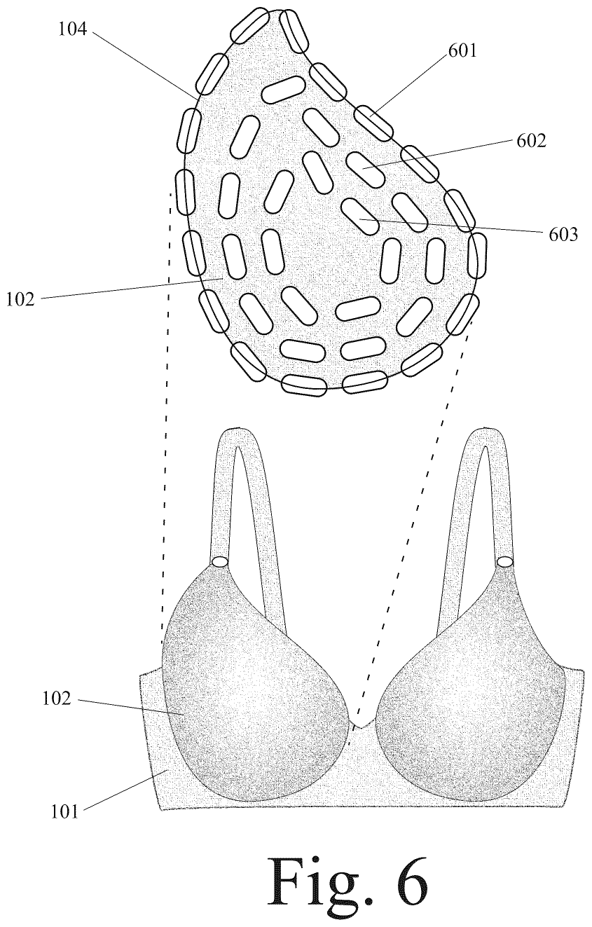

The lower portion of FIG. 6 shows an opaque frontal view of a smart bra 101 for optical detection of abnormal breast tissue, wherein the smart bra has two cups (including cup 102) which are configured to hold a person's breasts. The upper portion of FIG. 6 shows a close-up semi-transparent frontal view of this cup with multiple (e.g. three in this case) rings of optical modules, including outer ring 601, middle ring 602, and inner ring 603. Outer ring 601 is around the proximal perimeter 104 of the cup. In this example, proximal means closer to a chest wall and distal means farther from the chest wall. In this example, the three rings are nested from a semi-transparent frontal perspective even though they are not in the same two-dimensional plane. Relevant variations discussed elsewhere in this disclosure or in priority-linked disclosures can also be applied to this example.

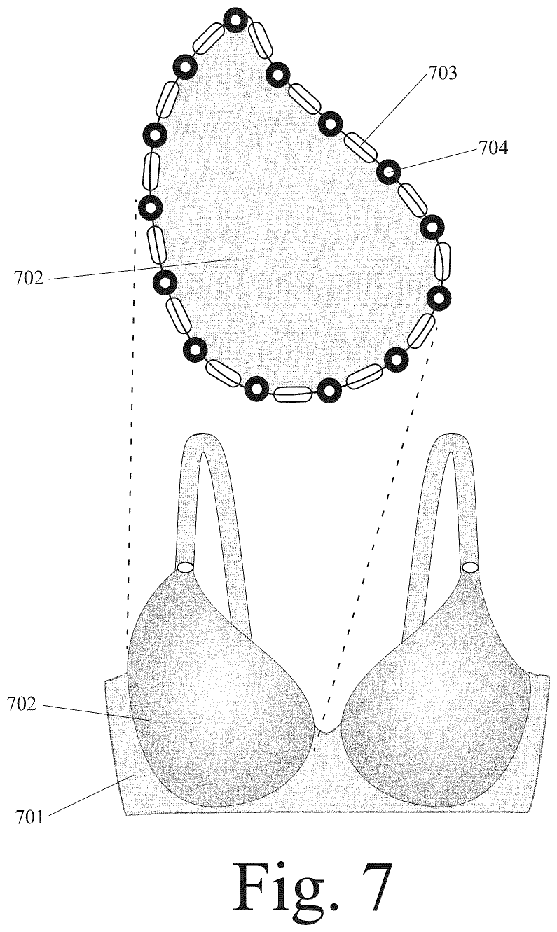

The lower portion of FIG. 7 shows an opaque frontal view of a smart bra 701 for optical detection of abnormal breast tissue, wherein the smart bra has two cups (including cup 702) which are configured to hold a person's breasts. The upper portion of FIG. 7 shows a close-up frontal view of a cross-section of this cup with optical modules (including optical module 703) and light receivers (including light receiver 704) around a perimeter of the cup. There can be additional optical modules and light receivers in other cross-sections of this cup even though they are not shown in this single cross-sectional view.

In an example, an optical module can include both a light emitter and a movable light guide. In an example, an optical module can include only a light emitter. A light emitter in an optical module transmits light into (and/or onto) breast tissue. A light receiver receives this light after the light has been transmitted through (and/or been reflected by) breast tissue. Changes in the light caused by interaction with the breast tissue are analyzed to image, detect, locate, and/or characterize abnormal breast tissue (e.g. tumors). In an example, a light receiver can receive light from a light emitter which is located in a different quadrant of the cup than the light receiver. Relevant variations discussed elsewhere in this disclosure or in priority-linked disclosures can also be applied to this example.

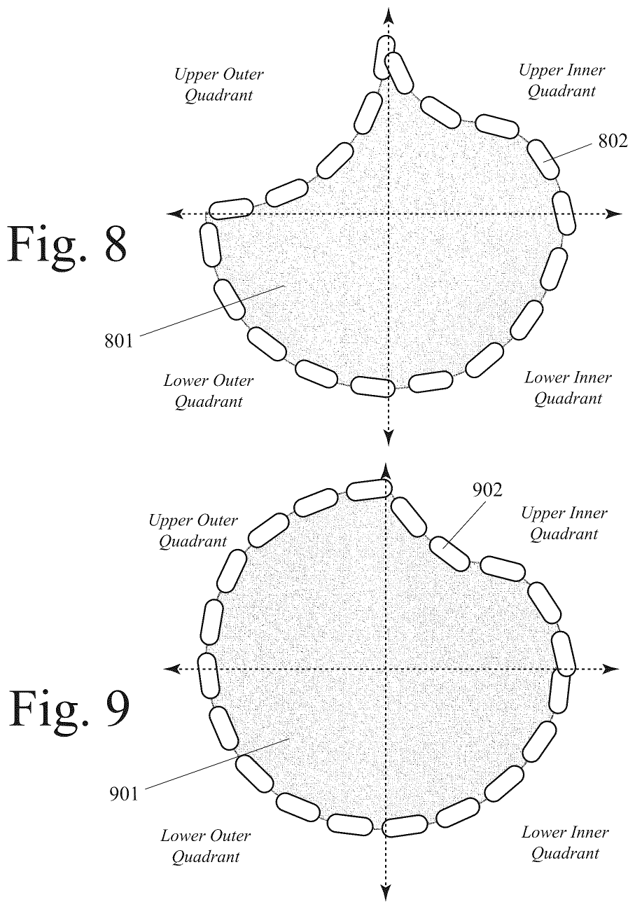

FIG. 8 shows a frontal cross-sectional view of a cup 801 of a smart bra for optical detection of abnormal breast tissue which includes a plurality of optical modules 802 around the perimeter of the cup. This figure shows the four quadrants of the cup: the upper-outer quadrant, the upper-inner quadrant, the lower-inner quadrant, and the lower-outer quadrant. In this example: the upper-outer quadrant of the perimeter has an outward-facing concavity; the upper-inner quadrant of the perimeter has a first section with an outward-facing concavity and a second section with an inward-facing concavity; the lower-inner quadrant of the perimeter has an inward-facing concavity; and the lower-outer quadrant of the perimeter has an inward-facing concavity. Relevant variations discussed elsewhere in this disclosure or in priority-linked disclosures can also be applied to this example.

FIG. 9 shows a frontal cross-sectional view of a cup 901 of a smart bra for optical detection of abnormal breast tissue which includes a plurality of optical modules 902 around the perimeter of the cup. This figure shows the four quadrants of the cup: the upper-outer quadrant, the upper-inner quadrant, the lower-inner quadrant, and the lower-outer quadrant. In this example: the upper-outer quadrant of the perimeter has an inward-facing concavity; the upper-inner quadrant of the perimeter has a first section with an outward-facing concavity and a second section with an inward-facing concavity; the lower-inner quadrant of the perimeter has an inward-facing concavity; and the lower-outer quadrant of the perimeter has an inward-facing concavity. Relevant variations discussed elsewhere in this disclosure or in priority-linked disclosures can also be applied to this example.

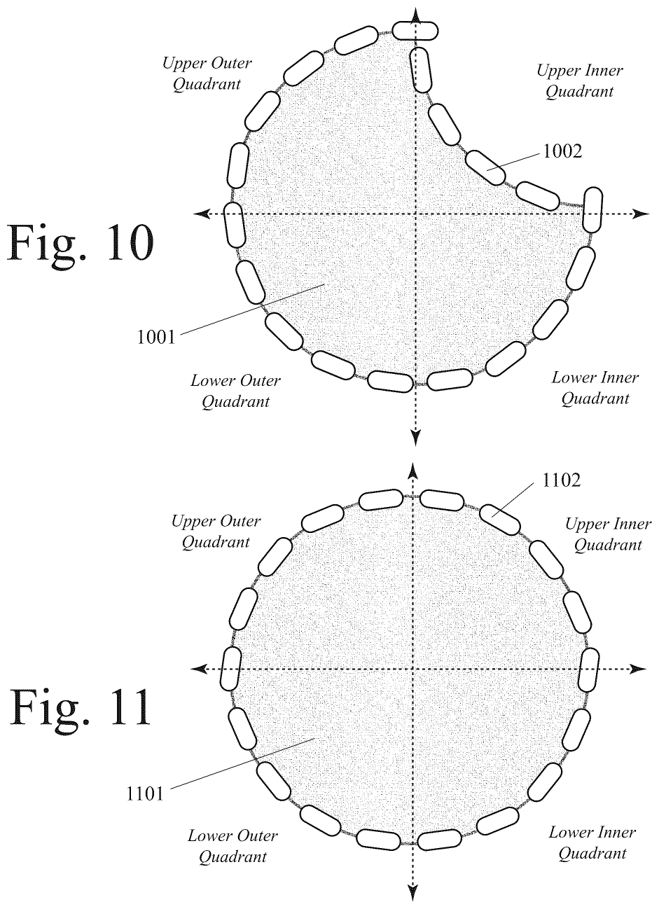

FIG. 10 shows a frontal cross-sectional view of a cup 1001 of a smart bra for optical detection of abnormal breast tissue which includes a plurality of optical modules 1002 around the perimeter of the cup. This figure shows the four quadrants of the cup: the upper-outer quadrant, the upper-inner quadrant, the lower-inner quadrant, and the lower-outer quadrant. In this example: the upper-outer quadrant of the perimeter has an inward-facing concavity; the upper-inner quadrant of the perimeter has an outward-facing concavity; the lower-inner quadrant of the perimeter has an inward-facing concavity; and the lower-outer quadrant of the perimeter has an inward-facing concavity. Relevant variations discussed elsewhere in this disclosure or in priority-linked disclosures can also be applied to this example.

FIG. 11 shows a frontal cross-sectional view of a cup 1101 of a smart bra for optical detection of abnormal breast tissue which includes a plurality of optical modules 1102 around the perimeter of the cup. This figure shows the four quadrants of the cup: the upper-outer quadrant, the upper-inner quadrant, the lower-inner quadrant, and the lower-outer quadrant. In this example: the upper-outer quadrant of the perimeter has an inward-facing concavity; the upper-inner quadrant of the perimeter has an inward-facing concavity; the lower-inner quadrant of the perimeter has an inward-facing concavity; and the lower-outer quadrant of the perimeter has an inward-facing concavity. Relevant variations discussed elsewhere in this disclosure or in priority-linked disclosures can also be applied to this example.

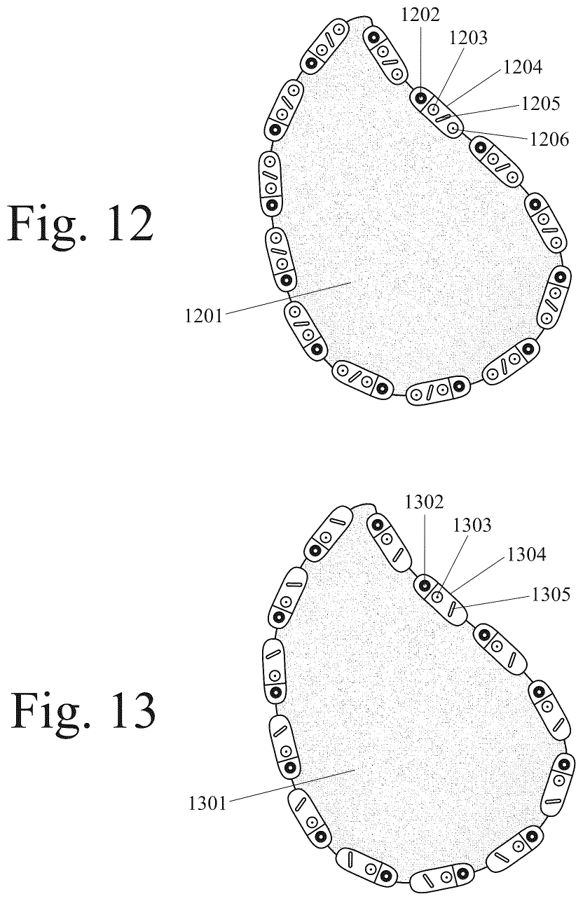

FIG. 12 shows a frontal cross-sectional view of a cup 1201 of a smart bra for optical detection of abnormal breast tissue comprising a plurality of optical modules (including optical module 1204) around the perimeter of the cup, wherein each optical module further comprises a first light emitter 1203, a second light emitter 1206, a movable light guide 1205, and a light receiver 1202.

In an example, a movable light guide can pivot and/or rotate. In an example, a movable light guide can reflect and/or refract light from the first light emitter and/or the second light emitter at different angles (or along different vectors) toward the breast held by the cup as the light guide pivots and/or rotates. In an example, a movable light guide can reflect and/or refract light from the first light emitter toward the breast when the light guide is in a first configuration and can reflect and/or refract light from the second light emitter toward the breast when the light guide is in a second configuration. In an example, a movable light guide can be a movable mirror (e.g. micromirror). In an example, the movable light guide can be a digital micromirror device.

In an example, a first light emitter can emit light at a first time and a second light emitter can emit light at a second time. In an example, a first light emitter can emit light with a first wavelength and a second light emitter can emit light with a second wavelength. In an example, a first light emitter can emit near-infrared light and a second light emitter can emit visible light. In an example, a first light emitter can emit near-infrared light and a second light emitter can emit red light. In an example, a first light emitter can emit near-infrared light and a second light emitter can emit green light. Relevant variations discussed elsewhere in this disclosure or in priority-linked disclosures can also be applied to this example.

FIG. 13 shows a frontal cross-sectional view of a cup 1301 of a smart bra for optical detection of abnormal breast tissue comprising a plurality of optical modules (including optical module 1304) around the perimeter of the cup, wherein each optical module further comprises a first light emitter 1305, a movable light guide 1303, and a light receiver 1302.

In an example, a movable light guide can pivot and/or rotate. In an example, ae movable light guide can reflect and/or refract light from the light emitter at different angles (or along different vectors) toward the breast held by the cup as the light guide pivots and/or rotates. In an example, a movable light guide can be a movable mirror (e.g. micromirror). In an example, the movable light guide can be a digital micromirror device. Relevant variations discussed elsewhere in this disclosure or in priority-linked disclosures can also be applied to this example.

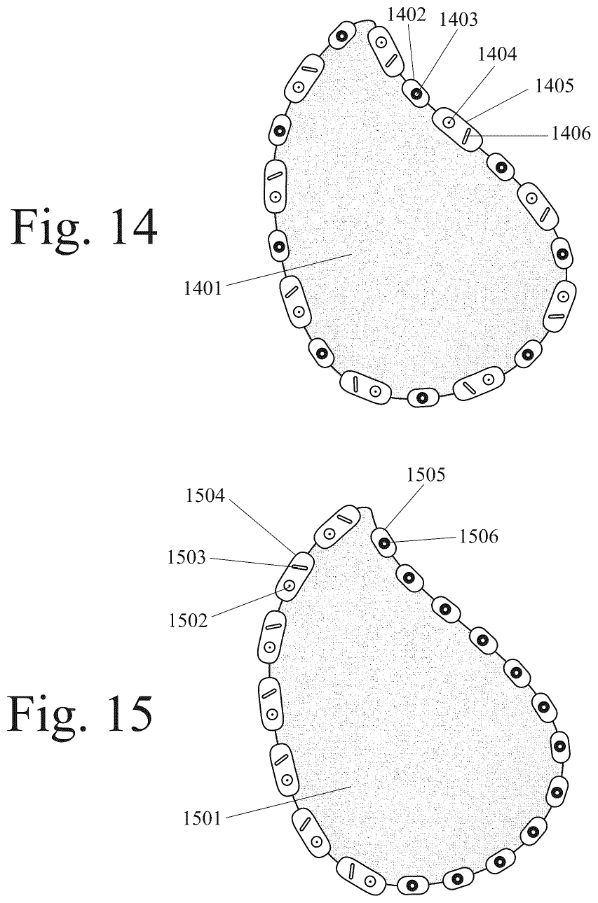

FIG. 14 shows a frontal cross-sectional view of a cup 1401 of a smart bra for optical detection of abnormal breast tissue comprising a plurality of optical modules around the perimeter of the cup, wherein optical modules (including optical module 1405) in a first set in the plurality each comprise a light emitter (such as light emitter 1404) and a movable light guide (such as light guide 1406), and wherein optical modules (including optical module 1402) in a second set in the plurality each comprise a light receiver (such as light receiver 1403). In this example, the plurality of optical modules comprise an alternating sequence of optical modules in the first set and optical modules in the second set.

In an example, a movable light guide can pivot and/or rotate. In an example, a movable light guide can reflect and/or refract light from a light emitter at different angles (or along different vectors) toward the breast held by the cup as the light guide pivots and/or rotates. In an example, a movable light guide can be a movable mirror (e.g. micromirror). In an example, a movable light guide can be a digital micromirror device. Relevant variations discussed elsewhere in this disclosure or in priority-linked disclosures can also be applied to this example.

FIG. 15 shows a frontal cross-sectional view of a cup 1501 of a smart bra for optical detection of abnormal breast tissue comprising a plurality of optical modules around the perimeter of the cup, wherein optical modules (including optical module 1504) in a first set in the plurality each comprise a light emitter (such as light emitter 1502) and a movable light guide (such as light guide 1503), and wherein optical modules (including optical module 1505) in a second set in the plurality each comprise a light receiver (such as light receiver 1506). In this example, the first set of optical modules are on a first side of the cup and the second set of optical modules are on the second side of the cup.

In an example, a movable light guide can pivot and/or rotate. In an example, a movable light guide can reflect and/or refract light from a light emitter at different angles (or along different vectors) toward the breast held by the cup as the light guide pivots and/or rotates. In an example, a movable light guide can be a movable mirror (e.g. micromirror). In an example, a movable light guide can be a digital micromirror device. Relevant variations discussed elsewhere in this disclosure or in priority-linked disclosures can also be applied to this example.

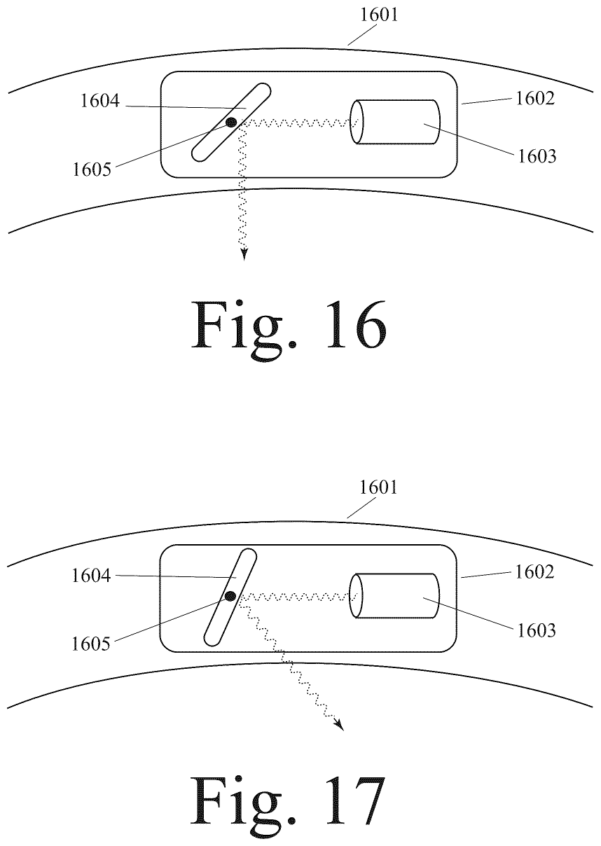

FIGS. 16 and 17 show two views, at two different times, of an optical module 1602 in a section 1601 of a perimeter of a cup of a smart bra for optical detection of abnormal breast tissue, wherein the optical module further comprises a light emitter 1603 and a movable light guide 1604 which pivots and/or rotates around an axle 1605. FIG. 16 shows this optical module at a first time when the movable light guide is in a first configuration (e.g. pivoted and/or rotated to a first angle) and light from the light emitter is directed toward breast tissue along a first vector. FIG. 17 shows this optical module at a second time when the movable light guide is in a second configuration (e.g. pivoted and/or rotated to a second angle) and light from the light emitter is directed toward breast tissue along a second vector.

In an example, the light guide can be a movable (micro) mirror. In an example, the light guide can be pivoted and/or rotated by changes in an electromagnetic field. In an example, the light guide can be pivoted and/or rotated by changes in an electromagnetic field created by electrodes in the optical module. In an example, the light guide can be a digital micromirror device. In an example, the light emitter can emit a beam of light toward the light guide along a vector which is substantially parallel to a side of the optical module and the light guide can redirect (e.g. reflect) this beam of light toward the breast. Relevant variations discussed elsewhere in this disclosure or in priority-linked disclosures can also be applied to this example.

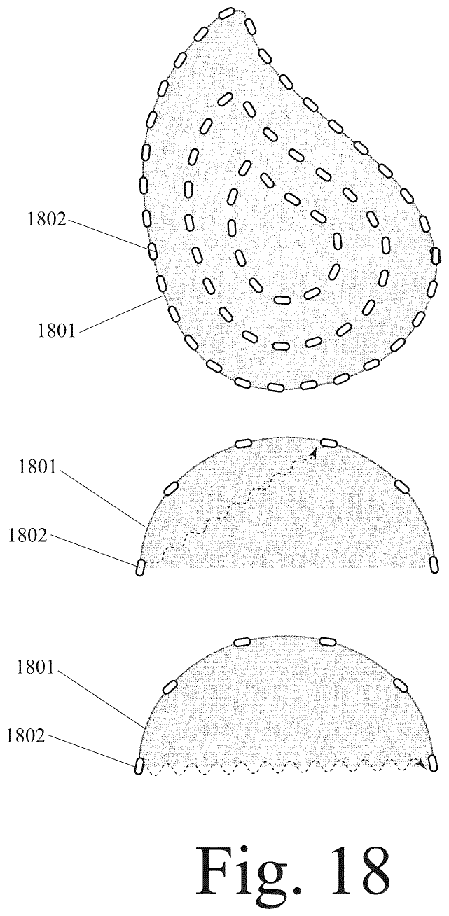

The upper portion of FIG. 18 shows a semi-transparent frontal view of a cup 1801 of a smart bra with nested rings of optical modules, including optical module 1802. The middle portion of FIG. 18 shows a lateral view of cross-section of this cup at a first time when optical module 1802 emits a beam of light into breast tissue at a first angle (e.g. along a first vector). The lower portion of FIG. 18 shows a lateral view of cross-section of this cup at a second time when optical module 1802 emits a beam of light into the breast tissue at a second angle (e.g. along a second vector). In an example, the angle at which the optical module emits a beam of light into breast tissue can be changed by a movable light guide (e.g. a pivoting and/or rotating mirror) within the optical module. Relevant variations discussed elsewhere in this disclosure or in priority-linked disclosures can also be applied to this example.

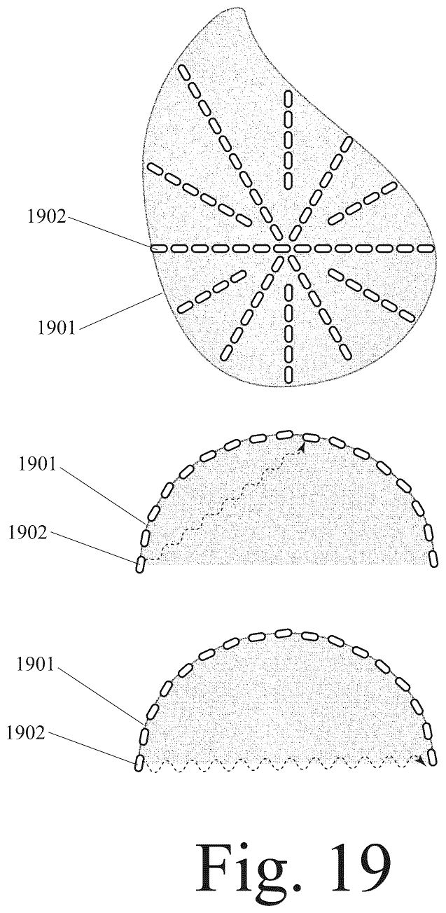

The upper portion of FIG. 19 shows a semi-transparent frontal view of a cup 1901 of a smart bra with a radial array (e.g. starburst or hub-and-spoke array) of optical modules, including optical module 1902. The middle portion of FIG. 19 shows a lateral view of cross-section of this cup at a first time when optical module 1902 emits a beam of light into breast tissue at a first angle (e.g. along a first vector). The lower portion of FIG. 19 shows a lateral view of cross-section of this cup at a second time when optical module 1902 emits a beam of light into the breast tissue at a second angle (e.g. along a second vector). In an example, the angle at which the optical module emits a beam of light into breast tissue can be changed by a movable light guide (e.g. a pivoting and/or rotating mirror) within the optical module. Relevant variations discussed elsewhere in this disclosure or in priority-linked disclosures can also be applied to this example.

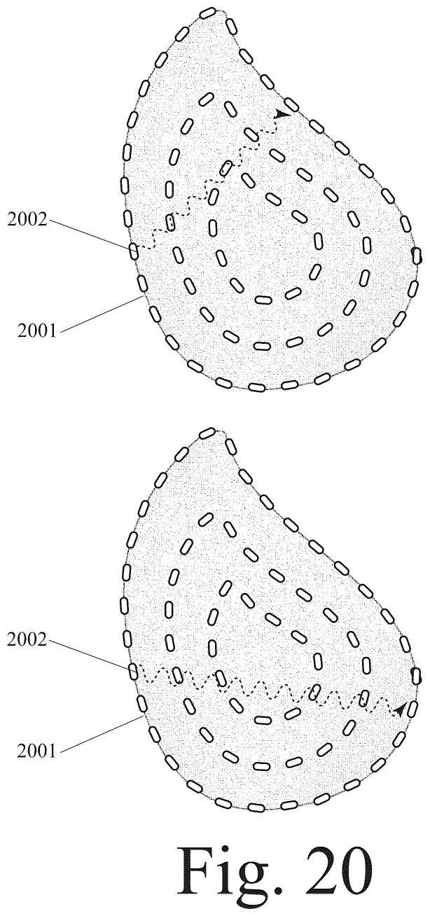

The upper portion of FIG. 20 shows a semi-transparent frontal view of a cup 2001 of a smart bra with nested rings of optical modules (including optical module 2002 on the outer ring) at a first time when the optical module 2002 emits a beam of light into breast tissue at a first angle (e.g. along a first vector). The lower portion of FIG. 20 shows a semi-transparent frontal view of the cup 2001 at a second time when the optical module 2002 emits a beam of light into the breast tissue at a second angle (e.g. along a second vector). In an example, the angle at which the optical module emits a beam of light into the breast tissue can be changed by a movable light guide (e.g. a pivoting and/or rotating mirror) within the optical module. Relevant variations discussed elsewhere in this disclosure or in priority-linked disclosures can also be applied to this example.

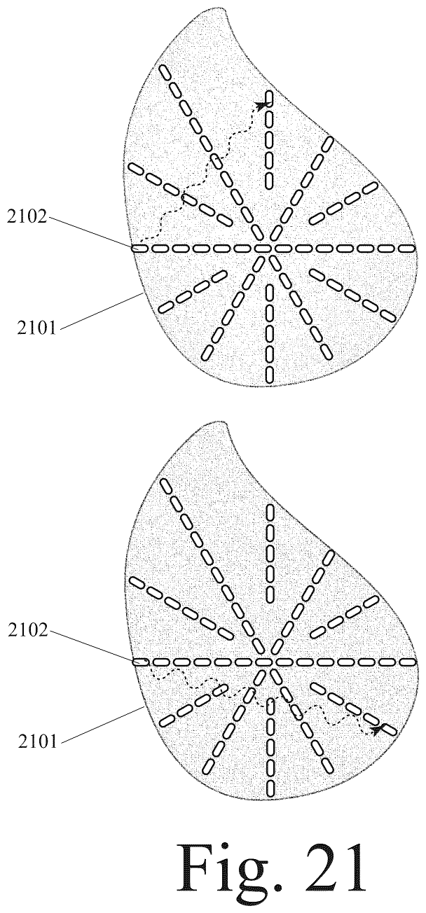

The upper portion of FIG. 21 shows a semi-transparent frontal view of a cup 2101 of a smart bra with a radial array (e.g. starburst or hub-and-spoke array) of optical modules, including optical module 2102, at a first time when the optical module 2102 emits a beam of light into breast tissue at a first angle (e.g. along a first vector). The lower portion of FIG. 21 shows a semi-transparent frontal view of the cup 2101 at a second time when the optical module 2102 emits a beam of light into the breast tissue at a second angle (e.g. along a second vector). In an example, the angle at which the optical module emits a beam of light into the breast tissue can be changed by a movable light guide (e.g. a pivoting and/or rotating mirror) within the optical module. Relevant variations discussed elsewhere in this disclosure or in priority-linked disclosures can also be applied to this example.

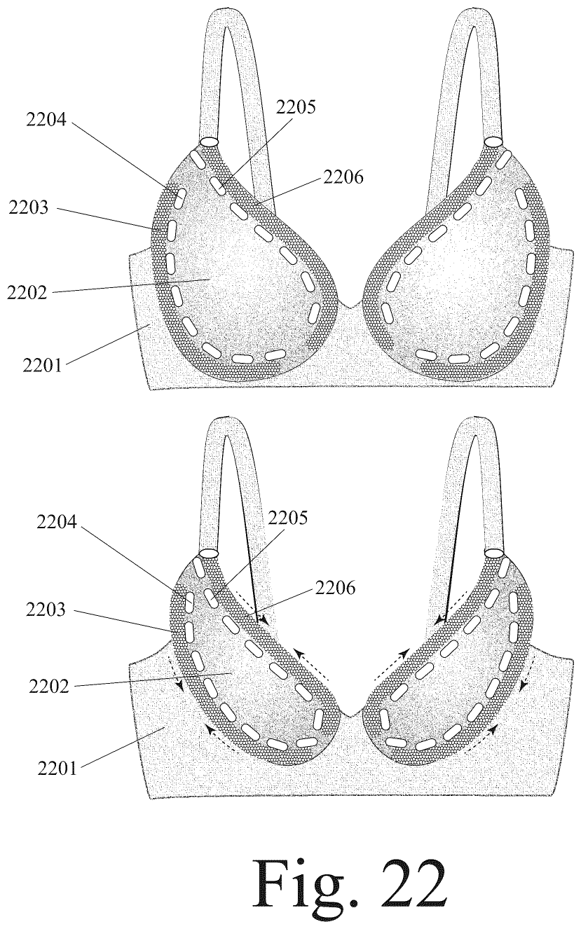

FIG. 22 shows two frontal (semi-transparent) views of a smart bra 2201 for optical detection of abnormal breast tissue including piezoelectric contracting bands for moderate compression of a breast and optical modules for optical scanning of the breast. The upper portion of FIG. 22 shows this smart bra at a first time, before the piezoelectric contracting bands have been contracted. The lower portion of FIG. 22 shows this smart bra at a second time, after the piezoelectric contracting bands have been contracted. This smart bra 2201 comprises: at least one cup 2202 which holds a person's breast; a first piezoelectric contracting band 2203 on a first side of the cup; a second piezoelectric contracting band 2206 on a second side (e.g. the opposite side) of the cup; a first set of optical modules (including 2204) on the first side of the cup; and a second set of optical modules (including 2205) on the second side of the cup.

In an example, the first side of the cup can be the right side of the cup and the second side of the cup can be the left side of the cup, wherein the two sides are separated by a vertical plane. In an example, the first side of the cup can be the upper side of the cup and the second side of the cup can be the lower side of the cup, wherein the two sides are separated by a horizontal plane. In an example, the first side of the cup can include the upper-inner quadrant of the cup and the second side of the cup can include the lower-outer quadrant of the cup, wherein the two sides are separated by an oblique plane (e.g. between vertical and horizontal). Relevant variations discussed elsewhere in this disclosure or in priority-linked disclosures can also be applied to this example.

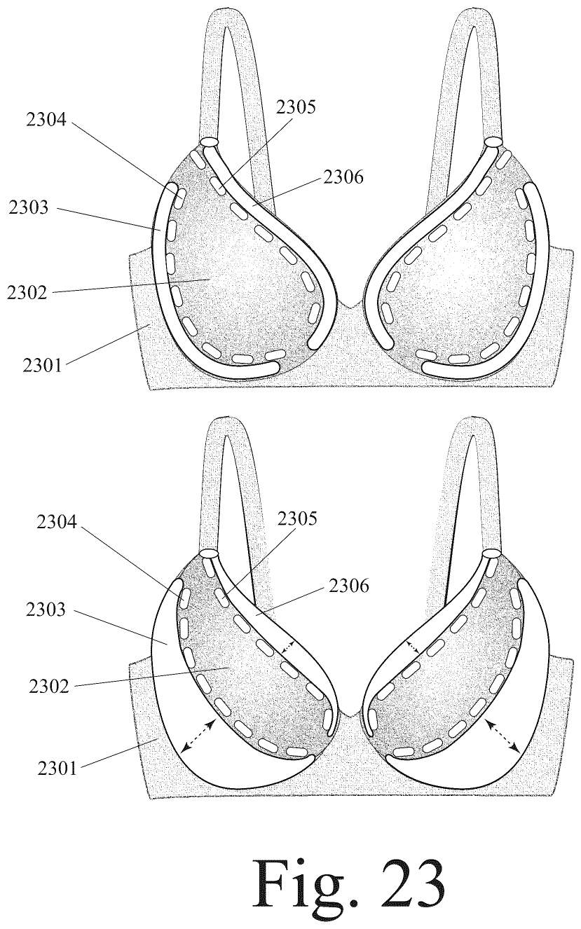

FIG. 23 shows two frontal (semi-transparent) views of a smart bra 2301 for optical detection of abnormal breast tissue including expandable chambers for moderate compression of a breast and optical modules for optical scanning of the breast. The upper portion of FIG. 23 shows this smart bra at a first time, before the expandable chambers have been expanded. The lower portion of FIG. 23 shows this smart bra at a second time, after the expandable chambers have been expanded. This smart bra 2301 comprises: at least one cup 2302 which holds a person's breast; a first expandable chamber 2303 on a first side of the cup; a second expandable chamber 2306 on a second side (e.g. the opposite side) of the cup; a first set of optical modules (including 2304) on the first side of the cup; and a second set of optical modules (including 2305) on the second side of the cup.

In an example, the first side of the cup can be the right side of the cup and the second side of the cup can be the left side of the cup, wherein the two sides are separated by a vertical plane. In an example, the first side of the cup can be the upper side of the cup and the second side of the cup can be the lower side of the cup, wherein the two sides are separated by a horizontal plane. In an example, the first side of the cup can include the upper-inner quadrant of the cup and the second side of the cup can include the lower-outer quadrant of the cup, wherein the two sides are separated by an oblique plane (e.g. between vertical and horizontal). Relevant variations discussed elsewhere in this disclosure or in priority-linked disclosures can also be applied to this example.

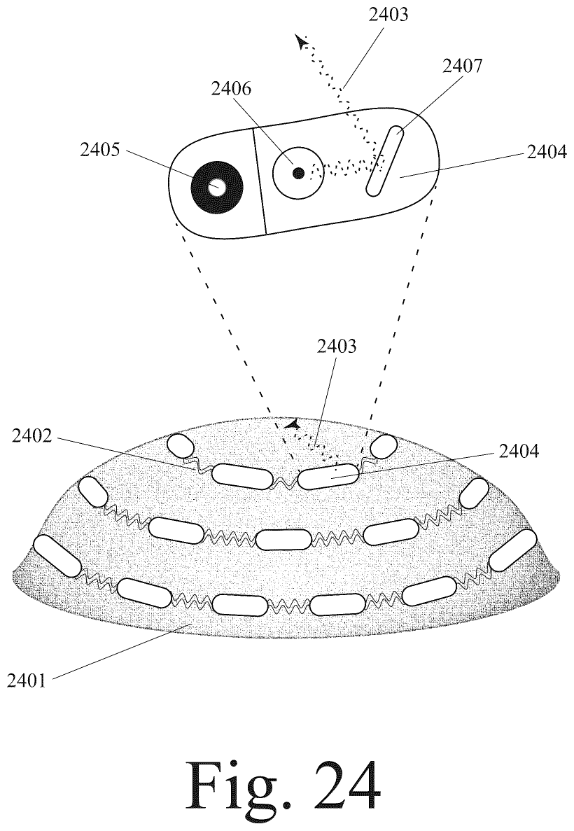

FIG. 24 shows an example of a smart bra cup 2401 for optical detection of abnormal breast tissue comprising: nested rings (e.g. three in this example) of optical modules (including 2404); and a plurality of undulating electroconductive pathways (including 2402) which connect and provide power to the optical modules; wherein each optical module further comprises a light emitter 2406 which emits a beam of light 2403, a movable light guide 2407 which redirects the beam of light toward a person's breast, and a light receiver 2405. The lower portion of FIG. 24 shows a lateral semi-transparent view of this smart bra cup. The upper portion of FIG. 24 shows a close-up interior view of one of the optical modules.

In an example, the undulating electroconductive pathways can be wires. In an example, the undulating electroconductive pathways can be made with a flexible and/or elastic polymer (e.g. PDMS) which has been doped, impregnated, and/or coated with electroconductive material (e.g. metal or carbon). In an example, the movable light guide can be a pivoting and/or rotating (micro) mirror. In an example, the movable light guide can be a digital micromirror device. Relevant variations discussed elsewhere in this disclosure or in priority-linked disclosures can also be applied to this example.

FIG. 25 shows an example of a smart bra cup 2501 for optical detection of abnormal breast tissue comprising: (three) nested rings of optical modules (including 2505); a plurality of undulating electroconductive pathways (including 2503) which connect and provide power to the optical modules; and (three) piezoelectric contracting rings and/or bands (including 2502); wherein each optical module further comprises a light emitter 2507 which emits a beam of light 2504, a movable light guide 2508 which redirects the beam of light toward a person's breast, and a light receiver 2506. The lower portion of FIG. 25 shows a lateral semi-transparent view of this smart bra cup. The upper portion of FIG. 25 shows a close-up interior view of one of the optical modules.

In an example, the undulating electroconductive pathways can be wires. In an example, the undulating electroconductive pathways can be made with a flexible and/or elastic polymer (e.g. PDMS) which has been doped, impregnated, and/or coated with electroconductive material (e.g. metal or carbon). In an example, the movable light guide can be a pivoting and/or rotating (micro) mirror. In an example, the movable light guide can be a digital micromirror device. Relevant variations discussed elsewhere in this disclosure or in priority-linked disclosures can also be applied to this example.

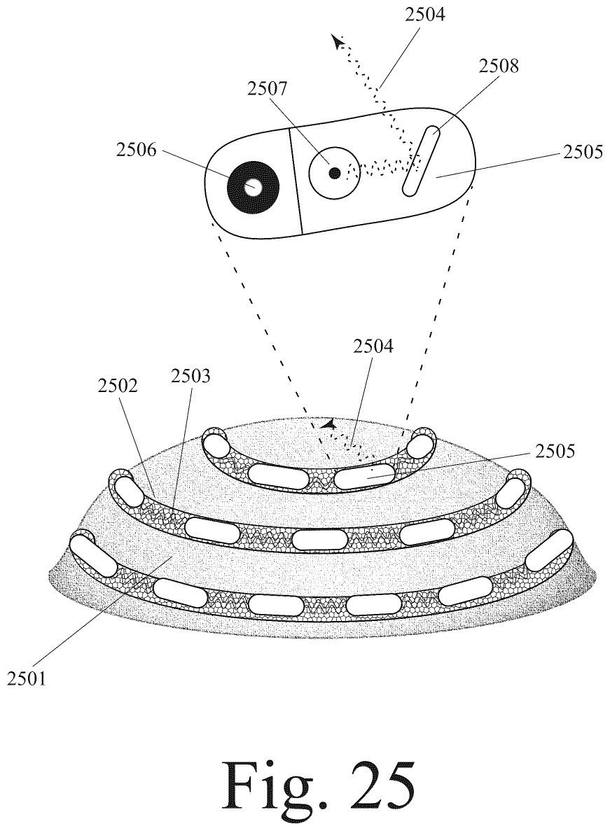

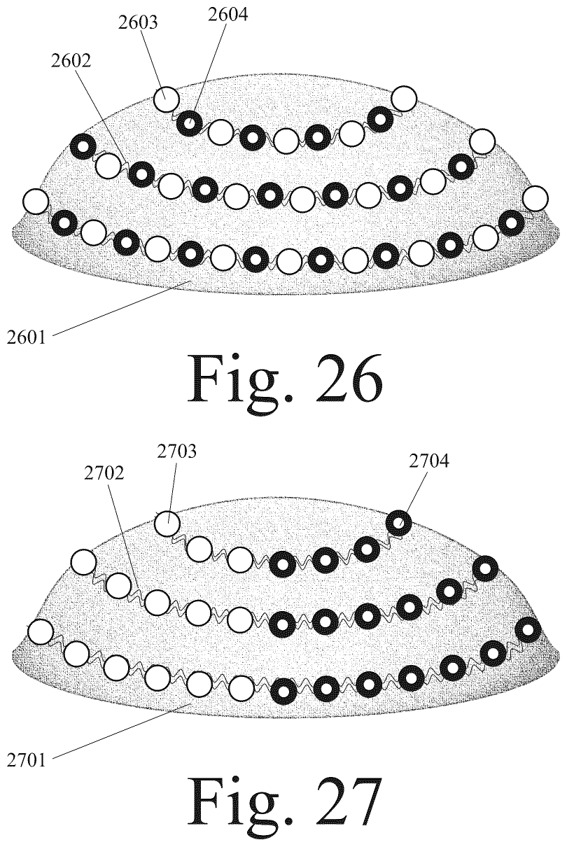

FIG. 26 shows a lateral semi-transparent view of a smart bra cup 2601 for optical detection of abnormal breast tissue comprising: (three) nested rings of light emitters (including 2603) and light receivers (including 2604); and undulating electroconductive pathways (including 2602) connected to the light emitters and light receivers. In this example, there is an alternating sequence of light emitters and light receivers around each of the nested rings. In an example, the electroconductive pathways can provide electrical power to the light emitters. In an example, the electroconductive pathways can also transmit data from the light receivers to a data processor. In an example, the electroconductive pathways can be wires. In an example, the electroconductive pathways can be made with a flexible and/or elastic polymer (e.g. PDMS) which has been doped, impregnated, and/or coated with electroconductive material (e.g. metal or carbon). Relevant variations discussed elsewhere in this disclosure or in priority-linked disclosures can also be applied to this example.

FIG. 27 shows a lateral semi-transparent view of a smart bra cup 2701 for optical detection of abnormal breast tissue comprising: (three) nested rings of light emitters (including 2703) and light receivers (including 2704); and undulating electroconductive pathways (including 2702) connected to the light emitters and light receivers.

In this example, light emitters are on a first side of a cup and light receivers are on a second side (e.g. opposite the first side) of the cup. In an example, the first side of a cup can be the right side of the cup and the second side of the cup can be the left side of the cup, wherein the first and second sides are separated by a vertical plane. In an example, the first side of a cup can be the upper side of the cup and the second side of the cup can be the lower side of the cup, wherein the first and second sides are separated by a horizontal plane. In an example, the first side of a cup can include the upper-inner quadrant of the cup and the second side of the cup can include the lower-outer quadrant of the cup, wherein the first and second sides are separated by an oblique plane between vertical and horizontal planes.

In an example, electroconductive pathways can provide electrical power to the light emitters. In an example, the electroconductive pathways can also transmit data from the light receivers to a data processor. In an example, the electroconductive pathways can be wires. In an example, the electroconductive pathways can be made with a flexible and/or elastic polymer (e.g. PDMS) which has been doped, impregnated, and/or coated with electroconductive material (e.g. metal or carbon). Relevant variations discussed elsewhere in this disclosure or in priority-linked disclosures can also be applied to this example.

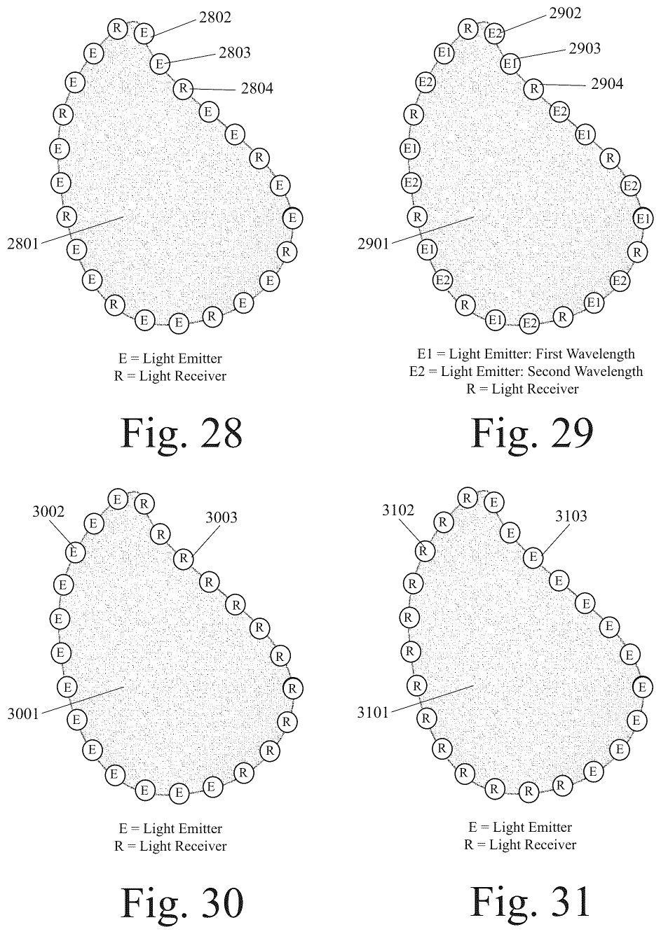

FIG. 28 shows a frontal view of a cross-section of a smart bra cup 2801 for optical detection of abnormal breast tissue comprising a sequence of light emitters (including 2802 and 2803) and light receivers (including 2804) which is distributed around the perimeter of the cross-section of the cup. In this example, this sequence is a repeating sequence of two light emitters and a light receiver. Relevant variations discussed elsewhere in this disclosure or in priority-linked disclosures can also be applied to this example.

FIG. 29 shows a frontal view of a cross-section of a smart bra cup 2901 for optical detection of abnormal breast tissue comprising a sequence of light emitters (including 2902 and 2903) and light receivers (including 2904) which is distributed around the perimeter of the cross-section of the cup. In this example, this sequence is a repeating sequence of two light emitters which emit light at different wavelengths and a light receiver. In an example, one light emitter in a repeating sequence can emit near-infrared light. In an example, one light emitter in a repeating sequence can emit visible light. In an example, one light emitter in a repeating sequence can emit red visible light. In an example, one light emitter in a repeating sequence can emit green visible light. Relevant variations discussed elsewhere in this disclosure or in priority-linked disclosures can also be applied to this example.

FIG. 30 shows a frontal view of a cross-section of a smart bra cup 3001 for optical detection of abnormal breast tissue comprising light emitters (including 3002) and light receivers (including 3003) which are around the perimeter of the cross-section of the cup. In this example, light emitters are on the outer (e.g. left) side of a cup and light receivers are on the inner (e.g. right) side of the cup, wherein light emitters and light receivers are separated by a vertical plane. In another example, light emitters can be on the upper side of the cup and light receivers can be on the lower side of the cup, or vice versa, wherein light emitters and light receivers are separated by a horizontal plane. In another example, light emitters can be on a first side of a cup which includes the upper-inner quadrant of the cup and light receivers can be on a second side of the cup which includes the lower-outer quadrant of the cup, or vice versa, wherein light emitters and light receivers are separated by an oblique plane between vertical and horizontal planes. Relevant variations discussed elsewhere in this disclosure or in priority-linked disclosures can also be applied to this example.

FIG. 31 shows a frontal view of a cross-section of a smart bra cup 3101 for optical detection of abnormal breast tissue comprising light emitters (including 3103) and light receivers (including 3102) which are around the perimeter of the cross-section of the cup. In this example, light emitters are on the inner (e.g. right) side of a cup and light receivers are on the outer (e.g. left) side of the cup, wherein light emitters and light receivers are separated by a vertical plane. In another example, light emitters can be on the lower side of the cup and light receivers can be on the upper side of the cup, or vice versa, wherein light emitters and light receivers are separated by a horizontal plane. In another example, light emitters can be on a first side of a cup which includes the lower-outer quadrant of the cup and light receivers can be on a second side of the cup which includes the upper-inner quadrant of the cup, or vice versa, wherein light emitters and light receivers are separated by an oblique plane between vertical and horizontal planes. Relevant variations discussed elsewhere in this disclosure or in priority-linked disclosures can also be applied to this example.

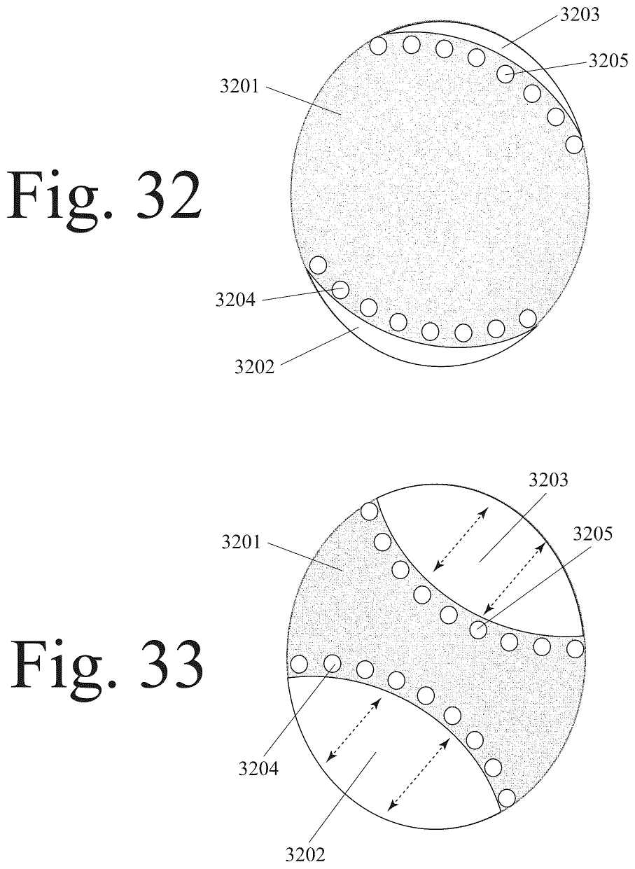

FIGS. 32 and 33 show two frontal views, at two different times, of a cross-section of a smart bra cup 3201 for optical detection of abnormal breast tissue comprising: a first set of optical modules (including 3204) on a first side of a bra cup; a first expandable chamber 3202 on the first side of the bra cup, wherein the first set of optical modules are configured to be located between the first expandable chamber and a breast held by the cup; a second set of optical modules (including 3205) on a second (e.g. opposite) side of the bra cup; and a second expandable chamber 3203 on the second side of the bra cup, wherein the second set of optical modules are configured to be located between the second expandable chamber and the breast.

FIG. 32 shows this smart bra cup at a first time when the expandable chambers are in a first configuration, before they have been expanded. FIG. 33 shows this smart bra cup at a second time when the expandable chambers are in a second configuration, after they have been expanded. In this example, the expandable chambers in their second configurations (after expansion) are shaped like convex lenses and/or footballs. In an example, the expandable chambers can be expanded by being filled with a flowable substance (e.g. a gas, liquid, or gel).

In an example, the first side of a cup can be the right side of the cup and the second side of the cup can be the left side of the cup, wherein the first and second sides are separated by a vertical plane. In an example, the first side of a cup can be the upper side of the cup and the second side of the cup can be the lower side of the cup, wherein the first and second sides are separated by a horizontal plane. In an example, the first side of a cup can include the upper-inner quadrant of the cup and the second side of the cup can include the lower-outer quadrant of the cup, wherein the first and second sides are separated by an oblique plane between vertical and horizontal planes.

In an example, an optical module can include a light emitter and a light receiver. In an example, some optical modules can include light emitters and some optical modules can include light receivers. In an example, a first set of optical modules can include light emitters and a second set of light emitters can include light receivers. In an example, an optical module can include a light emitter, a movable light guide, and a light receiver. Relevant variations discussed elsewhere in this disclosure or in priority-linked disclosures can also be applied to this example.

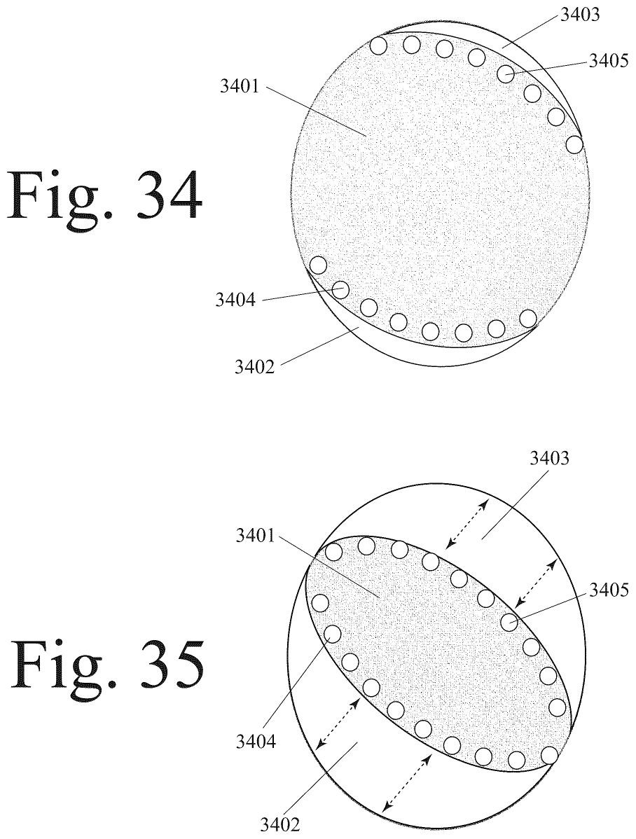

FIGS. 34 and 35 show two frontal views, at two different times, of a cross-section of a smart bra cup 3401 for optical detection of abnormal breast tissue comprising: a first set of optical modules (including 3404) on a first side of a bra cup; a first expandable chamber 3402 on the first side of the bra cup, wherein the first set of optical modules are configured to be located between the first expandable chamber and a breast held by the cup; a second set of optical modules (including 3405) on a second (e.g. opposite) side of the bra cup; and a second expandable chamber 3403 on the second side of the bra cup, wherein the second set of optical modules are configured to be located between the second expandable chamber and the breast.

FIG. 34 shows this smart bra cup at a first time when the expandable chambers are in a first configuration, before they have been expanded. FIG. 35 shows this smart bra cup at a second time when the expandable chambers are in a second configuration, after they have been expanded. In this example, the expandable chambers in their second configurations (after expansion) are shaped like crescents. In an example, the expandable chambers can be expanded by being filled with a flowable substance (e.g. a gas, liquid, or gel).

In an example, the first side of a cup can be the right side of the cup and the second side of the cup can be the left side of the cup, wherein the first and second sides are separated by a vertical plane. In an example, the first side of a cup can be the upper side of the cup and the second side of the cup can be the lower side of the cup, wherein the first and second sides are separated by a horizontal plane. In an example, the first side of a cup can include the upper-inner quadrant of the cup and the second side of the cup can include the lower-outer quadrant of the cup, wherein the first and second sides are separated by an oblique plane between vertical and horizontal planes.

In an example, an optical module can include a light emitter and a light receiver. In an example, some optical modules can include light emitters and some optical modules can include light receivers. In an example, a first set of optical modules can include light emitters and a second set of light emitters can include light receivers. In an example, an optical module can include a light emitter, a movable light guide, and a light receiver.

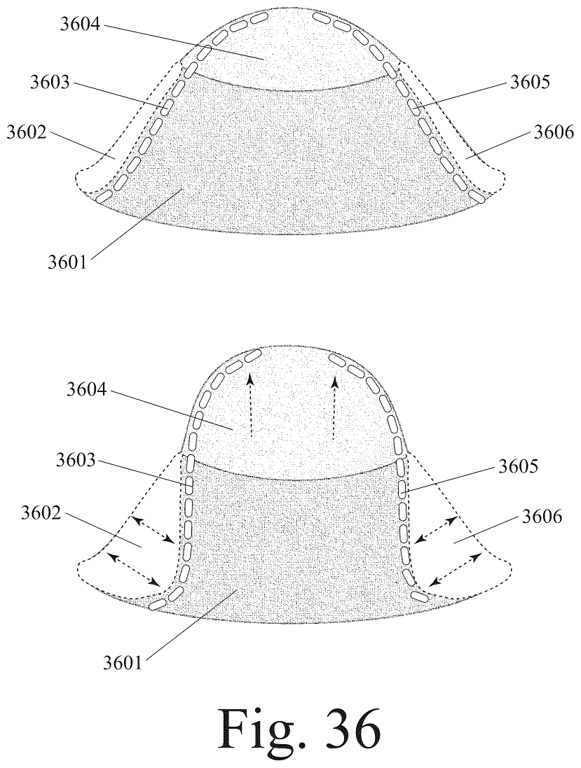

The upper and lower portions of FIG. 36 show lateral views, at two different times, of a cross-section of a smart bra cup for optical detection of abnormal breast tissue comprising: a proximal (e.g. closer to the chest wall) portion 3601 of the cup with a first level of elasticity or stretchability; a distal (e.g. farther from the chest wall) portion 3604 of the cup with a second level of elasticity or stretchability, wherein the second level is greater than the first level; a first set of optical modules (including 3603) on a first side of a bra cup; a first expandable chamber 3602 on the first side of the bra cup, wherein the first set of optical modules are configured to be located between the first expandable chamber and a breast held by the cup; a second set of optical modules (including 3605) on a second side of the bra cup; and a second expandable chamber 3606 on a second side of the bra cup, wherein the second set of optical modules are configured to be located between the second expandable chamber and the breast.

The upper portion of FIG. 36 shows this smart bra cup at a first time when the expandable chambers are in a first configuration, before they have been expanded. The lower portion of FIG. 36 shows this smart bra cup at a second time when the expandable chambers are in a second configuration, after they have been expanded. In an example, the expandable chambers can be expanded by being filled with a flowable substance (e.g. a gas, liquid, or gel). In this example, expansion of the expandable chambers compresses the lateral width of the breast. Reduction of the lateral width of the breast can improve the accuracy of optical scanning for detection of abnormal tissue. In this example, expansion of the expandable chambers also extends the breast in an anterior direction because the elastic and/or stretchable distal portion of the bra cup is more-easily extended than the proximal portion of the bra cup. Relevant variations discussed elsewhere in this disclosure or in priority-linked disclosures can also be applied to this example.

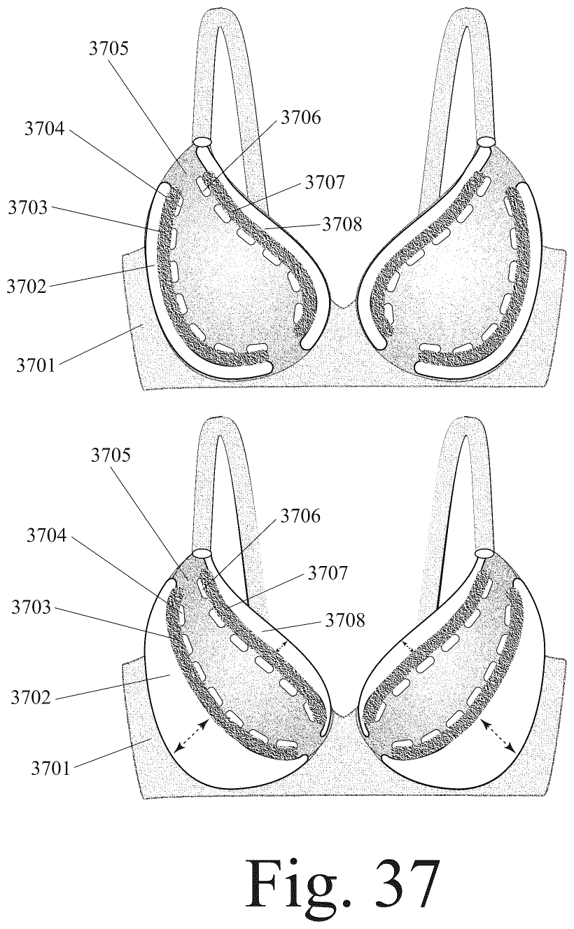

The upper and lower portions of FIG. 37 show frontal views, at two different times, of a cross-section of a smart bra cup for optical detection of abnormal breast tissue comprising: a first set of optical modules (including 3704) on a first side of a bra cup 3705; a first relatively-inelastic band and/or layer 3703 on the first side of the bra cup; a first expandable chamber 3702 on the first side of the bra cup; wherein the first set of optical modules are between the first relatively-inelastic band and/or layer and a breast held by the cup; wherein the first relatively-inelastic band and/or layer is between the first set of optical modules and the first expandable chamber; a second set of optical modules (including 3706) on a second side of the bra cup; a second relatively-inelastic band and/or layer 3707 on the second side of the bra cup; a second expandable chamber 3708 on the second side of the bra cup; wherein the second set of optical modules are between the second relatively-inelastic band and/or layer and the breast; and wherein the second relatively-inelastic band and/or layer is between the second set of optical modules and the second expandable chamber.

The upper portion of FIG. 37 shows this smart bra cup at a first time when the expandable chambers are in a first configuration, before they have been expanded. The lower portion of FIG. 37 shows this smart bra cup at a second time when the expandable chambers are in a second configuration, after they have been expanded. In an example, the expandable chambers can be expanded by being filled with a flowable substance (e.g. a gas, liquid, or gel). In this example, expansion of the expandable chambers compresses the lateral width of the breast.

In an example, the relatively-inelastic band and/or layer can help to make the thickness of the breast more uniform when compressed, thereby improving the accuracy of optical scanning of the breast for abnormal tissue. In an example, the relatively-inelastic band and/or layer can be relatively-inelastic compared to the rest of cup. In an example, the relatively-inelastic band and/or layer can have lower durometer level or lower Shore value than the rest of the cup. In an example, the relatively-inelastic band and/or layer can be made with a material which has a lower durometer level or lower Shore value than the material used to make the rest of the cup. In an example, the relatively-inelastic band and/or layer can be relatively-rigid compared to rest of the cup. Relevant variations discussed elsewhere in this disclosure or in priority-linked disclosures can also be applied to this example.

I claim:

1. A smart bra for optical detection of abnormal breast tissue comprising:
a bra with cups which are configured to hold a person's breasts;
wherein at least one of the cups has a plurality of optical modules;
wherein the plurality of optical modules further comprises light emitters and light receivers;
wherein light from the light emitters is received by the light receivers after the light has been transmitted through and/or reflected by breast tissue; and
wherein light received by the light receivers is analyzed to detect, locate, image, and/or characterize abnormal breast tissue.

2. The smart bra in claim 1 wherein the plurality of optical modules are arranged in rings.

3. The smart bra in claim 2 wherein the rings are nested from a frontal perspective.

4. The smart bra in claim 1 wherein the plurality of optical modules are arranged in a radial array.

5. The smart bra in claim 4 wherein the radial array is a starburst array.

6. The smart bra in claim 4 wherein the radial array is a hub-and-spoke array.

7. The smart bra in claim 1 wherein the plurality of optical modules are arranged in a honeycomb grid.

8. The smart bra in claim 1 wherein optical modules in the plurality of optical modules each have both a light emitter and a light receiver.

9. The smart bra in claim 1 wherein optical modules in the plurality of optical modules each have two light emitters and a light receiver.

10. The smart bra in claim 9 wherein the two light emitters emit light with different wavelengths.

11. The smart bra in claim 9 wherein the two light emitters emit light at different times.

12. The smart bra in claim 1 wherein a first set of optical modules in the plurality of optical modules each have a light emitter but no light receiver and wherein a second set of optical modules in the plurality of optical modules each have a light receiver but no light emitter.

13. The smart bra in claim 1 wherein each optical module in the plurality of optical modules has a light emitter and a movable light guide.

14. The smart bra in claim 13 wherein the movable light guide reflects and/or refracts a beam of light from a light emitter toward a breast.

15. The smart bra in claim 13 wherein the light guide is a movable mirror.

16. The smart bra in claim 1 wherein each optical module in the plurality of optical modules has a light emitter, a movable light guide, and a light receiver.

17. The smart bra in claim 1 wherein each optical module in the plurality of optical modules has a first light emitter, a second light emitter, a movable light guide, and a light receiver.

18. The smart bra in claim 17 wherein the movable light guide is located between the first light emitter and the second light emitter.

19. The smart bra in claim 1 wherein the smart bra further comprises a plurality of expandable chambers, wherein expansion of the expandable chambers compresses a breast.

20. The smart bra in claim 1 wherein the smart bra further comprises a plurality of piezoelectric contracting bands or layers, wherein contraction of the piezoelectric contracting bands or layers compresses a breast.
